(19) United States
(12) Patent Application Publication
(10) Pub. No.: US 2017/0174783 A1
(43) Pub. Date: Jun. 22, 2017

(54) Single-Arm Monovalent Antibody Constructs and Uses Thereof

(71) Applicant: Zymeworks Inc., Vancouver (CA)

(72) Inventors: Gordon Yiu Kon NG, Vancouver (CA); Surjit Bhimarao DIXIT, Richmond (CA); Thomas SPRETER VON KREUDENSTEIN, Vancouver (CA)

(21) Appl. No.: 15/298,625

(22) Filed: Oct. 20, 2016

Related U.S. Application Data
(63) Continuation of application No. 14/399,789, filed on Nov. 7, 2014, now abandoned.
(60) Provisional application No. 61/645,547, filed on May 10, 2012.
(60) Provisional application No. 61/671,640, filed on Jul. 13, 2012.
(60) Provisional application No. 61/722,070, filed on Nov. 2, 2012.
(60) Provisional application No. 61/762,812, filed on Feb. 8, 2013.

(51) Int. Cl. C07K 16/32; C12P 21/02

(52) U.S. Cl. CPC C07K 16/32; C12P 21/02; C07K 2317/35; C07K 2317/55; C07K 2317/622; C07K 2317/52; C07K 2317/41; C07K 2317/92; C07K 2317/732; C07K 2317/734; C07K 2317/73; C07K 2317/77; C07K 2317/94; A61K 2039/505

(57) ABSTRACT

Provided herein are monovalent antibody constructs. In specific embodiments is a monovalent antibody construct comprising: an antigen-binding polypeptide construct which monovalently binds an antigen; and a dimeric Fc polypeptide construct comprising a CH3 domain, said construct comprising two monomeric Fc polypeptides, wherein one said monomeric Fc polypeptide is fused to at least one polypeptide from the antigen-binding polypeptide construct. These therapeutically novel molecules encompass monovalent constructs that display an increase in binding density and Bmax (maximum binding at a target to antibody ratio of 1:1) to a target cell displaying said antigen as compared to a corresponding monospecific bivalent antibody construct with two antigen binding regions. Provided herein are methods for creation of monovalent antibody constructs that shows superior effector efficacy as compared to the corresponding bivalent antibody construct at equimolar concentrations. Provided herein are methods for creation of monovalent antibody constructs that unexpectedly inhibit tumor cell growth and can be internalized and show greater efficacy compared to a bivalent antibody construct at equimolar saturating concentrations. Provided are monovalent antibody constructs for the treatment of HER2 expressing diseases.

### CROSS REFERENCE TO RELATED APPLICATIONS

This application is a continuation of U.S. application Ser. No. 14/399,789, filed Nov. 7, 2014, which claims benefit to application PCT/CA2013/050358, filed May 8, 2013, which claims the benefit under 35 U.S.C. §1 19(e) of U.S. Provisional Patent Application No. 61/645,547, filed May 10, 2012; U.S. Provisional Patent Application No. 61/722,070, filed Nov. 2, 2012; U.S. Provisional Patent Application No. 61/671,640, filed Jul. 13, 2012 and U.S. Provisional Patent Application No. 61/762,812, filed Feb. 8, 2013, each of which is herein incorporated by reference in its entirety, for all purposes.

### REFERENCE TO SEQUENCE LISTING

The instant application contains a Sequence Listing which has been submitted in ASCII format via EFS-Web and is hereby incorporated by reference in its entirety. Said ASCII copy, created on Oct. 20, 2016, is named 35246_US_sequence_listing.txt and is 135 kilobytes in size.

### FIELD OF INVENTION

The field of the invention is the rational design of a scaffold for custom development of biotherapeutics.

### DESCRIPTION OF RELATED ART

In the realm of therapeutic proteins, antibodies with their multivalent target binding features are excellent scaffolds for the design of drug candidates. Current marketed antibody therapeutics are bivalent monospecific antibodies optimized and selected for high affinity binding and avidity conferred by the two antibody FABs. Defucosylation or enhancement of FcgR binding by mutagenesis have been employed to render antibodies more efficacious via antibody Fc dependent cell cytotoxicity mechanisms. Afucyosylated antibodies or antibodies with enhanced FcgR binding still suffer from incomplete therapeutic efficacy in clinical testing and marketed drug status has yet to be achieved for any of these antibodies.

Therapeutic antibodies would ideally possess certain minimal characteristics, including target specificity, biostability, bioavailability and biodistribution following administration to a subject patient, and sufficient target binding affinity and high target occupancy and antibody decoration of target cells to maximize antibody dependent therapeutic effects. There has been limited success in efforts to generate antibody therapeutics that possess all of these minimal characteristics especially antibodies that can fully occupy targets at a 1:1 antibody to target ratio. For example, full length bivalent monospecific IgG antibodies can not fully occupy targets at a 1:1 ratio even at saturating concentrations. From a theoretical perspective, at saturating concentrations a traditional monospecific bivalent antibody is expected to maximally binds targets at a ratio of 1 antibody:2 targets owing to the presence of two identical antigen binding FABs that can confer avidity effects compared to monovalent antibody fragments. Further, such full length antibodies suffer from more limited bioavailability and/or biodistribution as a consequence of greater molecular size. Furthermore, a full length antibody may in some cases exhibit agonistic effects upon binding to a target antigen, which is undesired in instances where the antagonistic effect is the desired therapeutic function. In some instances, this phenomenon is attributable to the “cross-linking” effect of a bivalent antibody that when bound to a cell surface receptor promotes receptor dimerization that leads to receptor activation. Additionally, traditional bivalent antibodies suffer from limited therapeutic efficacy because of limited antibody binding and decoration of target cells at a 1:2 antibody to target antigen ratio at maximal therapeutically safe doses that permit antibody dependent cytotoxic effects or other mechanisms of therapeutic activity.

### SUMMARY OF THE INVENTION

Provided herein is an isolated monovalent antibody construct comprising: an antigen-binding polypeptide construct which monovalently binds an antigen; and a dimeric Fc polypeptide construct, said Fc polypeptide construct comprising two monomeric Fc polypeptides each comprising a CH3 domain, wherein one said monomeric Fc polypeptide is fused to at least one polypeptide from the antigen-binding polypeptide construct; wherein said monovalent antibody construct selectively and/or specifically binds a target cell displaying said antigen with: an increased binding density and Bₘₐₓ as compared to a corresponding monospecific bivalent antibody construct with two antigen binding regions; a dissociation constant (K_{d}) comparable to said monospecific bivalent antibody construct; an off-rate that is comparable or slower that said monospecific bivalent antibody construct; and wherein said monovalent antibody construct displays biophysical and in vivo stability comparable to said monospecific bivalent antibody construct; and cytotoxicity comparable to or greater than said monospecific bivalent antibody construct.

In certain embodiments is provided the isolated monovalent antibody construct described herein, wherein the monovalent antibody construct blocks binding of the cognate ligand to the target antigen. In certain embodiments is the isolated monovalent antibody construct provided herein, wherein the monovalent antibody construct does not block binding of the cognate ligand to the target antigen. In an embodiment is the isolated monovalent antibody construct, wherein at an antibody to target ratio of 1:1 the increase in binding density and Bmax relative to a monospecific bivalent antibody, is observed at a concentration greater than the observed equilibrium constant (Kd) of the antibodies up to saturating concentrations. In an embodiment is the isolated monovalent antibody construct described herein, wherein said monovalent antibody construct displays at least one of higher ADCC, higher ADCP and higher CDC efficacy as compared to said corresponding bivalent antibody construct at a concentration greater than the observed equilibrium constant (Kd) of the antibodies up to saturating concentrations.

Provided in some embodiments is the isolated monovalent antibody construct described herein, wherein said construct is a monovalent lytic antibody construct that comprises an Fc domain that engages in effector activity, wherein said lytic antibody construct is non-agonistic, blocks cognate ligand binding to the target antigen, inhibits cell growth; and wherein said lytic antibody construct binds and saturates said target cell with increased Bₘₐₓ, fast on-rate and a comparable off-rate as compared to a corresponding monospecific bivalent antibody construct with two antigen binding regions.

In an embodiment is the isolated monovalent antibody construct, wherein said construct is not internalized. In some embodiments is the isolated monovalent antibody construct, wherein said construct is internalized.

Provided herein is an isolated monovalent antibody construct described herein, wherein said construct is a monovalent internalizing antibody construct that is effectively internalized; wherein said internalizing antibody is non-agonistic, blocks cognate ligand binding to the target antigen, and does not induce cell growth; and wherein said internalizing antibody construct binds said target cell with increased Bₘₐₓ, fast on-rate and a slower off-rate as compared to a corresponding monospecific bivalent antibody construct with two antigen binding regions.

In an embodiment is the isolated monovalent antibody construct described herein, wherein the internalization of said construct is greater than, equal to or less than that of the corresponding monospecific bivalent antibody. In an embodiment is the isolated monovalent antibody construct described herein, wherein said increase in binding density and Bmax is independent of the density of the antigen on the target cell. In an embodiment is provided the isolated monovalent antibody construct described herein, wherein said increase in binding density and Bmax is independent of the target antigen epitope.

In an embodiment is the isolated monovalent antibody construct described herein, wherein said construct exhibits no avidity

In an embodiment is the isolated monovalent antibody construct described herein, wherein said dimeric Fc polypeptide construct is heterodimeric. In an embodiment is the isolated monovalent antibody construct described herein wherein said monovalent antigen binding polypeptide construct is a Fab fragment, an scFv, an sdAb, an antigen binding peptide or a protein domain capable of binding the antigen. In one embodiment is the isolated monovalent antibody construct wherein said Fab fragment comprises a heavy chain polypeptide and a light chain polypeptide.

In an embodiment is the isolated monovalent antibody construct described herein, wherein the target cell is a cell expressing the cognate antigen, said cell selected from a list comprising: a cancer cell, and a diseased cell expressing HER2. In some embodiments is the isolated monovalent antibody construct described herein, wherein said antigen-binding polypeptide construct binds HER2 and wherein the target cell is at least one of: a low, medium or high HER2 expressing cell, a progesterone receptor negative cell or an estrogen receptor negative cell. In an embodiment is the isolated monovalent antibody construct described herein wherein said antigen-binding polypeptide construct binds a HER2 extra-cellular domain wherein said extra cellular domain is at least one of ECR 1, 2, 3, and 4.

Provided herein is an isolated monovalent antibody construct that binds HER2 comprising: an antigen binding polypeptide construct which monovalently binds HER2; and a dimeric Fc polypeptide construct comprising two monomeric Fc polypeptides each comprising a CH3 domain, wherein one of said monomeric Fc polypeptide is fused to the antigen-binding polypeptide construct; wherein said antibody construct displays an increase in binding density to FCγR compared to a corresponding bivalent antibody construct which binds HER2 at equimolar concentrations.

Provided herein is an isolated monovalent antibody construct that binds HER2 comprising: an antigen binding polypeptide construct which monovalently binds HER2; and a dimeric Fc polypeptide construct comprising two monomeric Fc polypeptides each comprising a CH3 domain, wherein one of said monomeric Fc polypeptide is fused to the antigen-binding polypeptide construct; wherein said antibody construct is internalized by a target cell, wherein said construct displays an increase in binding density and Bmax to HER2 displayed on the target cell as compared to a corresponding bivalent antibody construct which binds HER2, and wherein said construct displays at least one of higher ADCC, higher ADCP and higher CDC as compared to said corresponding bivalent HER2 binding antibody constructs at equimolar concentrations.

In an embodiment is an isolated monovalent antibody construct that binds HER2 comprising: an antigen binding polypeptide construct which monovalently binds HER2; and a dimeric Fc polypeptide construct comprising two monomeric Fc polypeptides each comprising a CH3 domain, wherein one of said monomeric Fc polypeptide is fused to the antigen-binding polypeptide construct; wherein said antibody construct binds FcRn but displays higher Vss compared to a corresponding monospecific bivalent antibody construct with two antigen binding regions.

In some embodiments is an isolated monovalent HER2 binding antibody construct described herein, wherein said monovalent HER2 binding polypeptide construct is at least one of Fab, an scFv, an sdAb, or a polypeptide.

Provided herein is an isolated monovalent antibody construct described herein, wherein the dimeric Fc construct is a heterodimeric Fc construct comprising a variant CH3 domain. In an embodiment is the isolated monovalent antibody construct described herein, said variant CH3 domain comprising amino acid mutations that promote the formation of said heterodimer with stability comparable to a native homodimeric Fc region. In an embodiment is the isolated monovalent antibody construct, wherein the variant CH3 domain has a melting temperature (Tm) of about 70° C. or higher. In a further embodiment is the isolated monovalent antibody, wherein the variant CH3 domain has a melting temperature (Tm) of about 75° C. or higher. Also provided is an isolated monovalent antibody construct described herein, wherein the variant CH3 domain has a melting temperature (Tm) of about 80° C. or higher. In a further embodiment is the isolated monovalent antibody construct described herein, wherein the dimeric Fc construct further comprises a variant CH2 domain comprising amino acid modifications to promote selective binding of Fcgamma receptors. In a related embodiment is the isolated monovalent antibody construct described herein, wherein the heterodimer Fc construct does not comprise an additional disulfide bond in the CH3 domain relative to a wild type Fc region. In an embodiment is the isolated monovalent antibody construct provided herein wherein the heterodimer Fc construct comprises an additional disulfide bond in the variant CH3 domain relative to a wild type Fc region, and wherein the variant CH3 domain has a melting temperature (Tm) of at least about 77.5° C. In an embodiment is the isolated monovalent antibody construct described herein wherein the dimeric Fc construct is a heterodimeric Fc construct formed with a purity greater than about 75%. In some embodiments is the isolated monovalent antibody described herein wherein the dimeric Fc construct is a heterodimeric Fc construct formed with a purity greater than about 80%. Also provided is the isolated monovalent antibody construct wherein the dimeric Fc construct is a heterodimeric Fc construct formed with a purity greater than about 90%. In some embodiments is the isolated monovalent antibody construct described herein wherein the dimeric Fc construct is a heterodimeric Fc construct formed with a purity greater than about 95%.

Provided herein is an isolated monovalent antibody construct described herein, wherein said monomeric Fc polypeptide is fused to the antigen-binding polypeptide construct by a linker. In certain embodiments, the linker is a polypeptide linker.

Provided in an embodiment is the isolated monovalent antibody construct described herein, wherein said construct possesses greater than about 105% of at least one of the ADCC, ADCP and CDC of a corresponding bivalent antibody construct with two antigen binding polypeptide construct. In an embodiment is a construct that possesses at least about 125% of at least one of the ADCC, ADCP and CDC of a corresponding bivalent antibody construct with two antigen binding polypeptide construct. In another embodiment is a construct that possesses at least about 150% of at least one of the ADCC, ADCP and CDC of a corresponding bivalent antibody construct. In an embodiment is the isolated monovalent antibody construct described herein, wherein said construct possesses at least about 300% of at least one of the ADCC, ADCP and CDC of a corresponding bivalent antibody construct with two antigen binding polypeptide construct.

Provided in an embodiment is the isolated monovalent antibody construct described herein, wherein said increase in binding density and Bₘₐₓ is at least about 125% of the binding density and Bmax of the corresponding bivalent antibody construct. In an embodiment is the isolated monovalent antibody construct described herein, wherein said increase in binding density and Bₘₐₓ is at least about 150% of the binding density and Bmax of the corresponding bivalent antibody construct. Also provided is the isolated monovalent antibody construct described herein, wherein said increase in binding density and Bₘₐₓ is at least about 200% of the binding density and Bmax of the corresponding bivalent antibody construct.

In an embodiment is a host cell comprising nucleic acid encoding the isolated monovalent antibody construct described herein. In some embodiments a host cell, wherein the nucleic acid encoding the antigen binding polypeptide construct and the nucleic acid encoding the Fc construct are present in a single vector. Also provided is a method of preparing an isolated monovalent antibody construct described herein, the method comprising the steps of: (a) culturing a host cell comprising nucleic acid encoding the antibody fragment; and (b) recovering the antibody fragment from the host cell culture.

In an embodiment is a method of producing a glycosylated monovalent antibody construct or a or glycoengineer afucosylated monovalent antibody construct in stable mammalian cells, comprising: transfecting at least one stable mammalian cell with: a first DNA sequence encoding a first heavy chain polypeptide comprising a heavy chain variable domain and a first Fc domain polypeptide; a second DNA sequence encoding a second heavy chain polypeptide comprising a second Fc domain polypeptide, wherein said second heavy chain polypeptide is devoid of a variable domain; and a third DNA sequence encoding a light chain polypeptide comprising a light chain variable domain, such that the said first DNA sequence, said second DNA sequence and said third DNA sequences are transfected in said mammalian cell in a pre-determined ratio; translating the said first DNA sequence, said second DNA sequence, and said third DNA sequence in the at least one mammalian cell such that said heavy and light chain polypeptides are expressed as the desired glycosylated monovalent asymmetric antibody in said at least one stable mammalian cell.

Provided in an embodiment is the method of producing a glycosylated monovalent antibody construct or a or glycoengineer afucosylated monovalent antibody construct described herein, comprising transfecting at least two different cells with different pre-determined ratios of said first DNA sequence, said second DNA sequence and said third DNA sequence such that each of the at least two cells expresses the heavy chain polypeptides and the light chain polypeptide in a different ratio. In an embodiment is the method of producing a glycosylated monovalent antibody construct or a or glycoengineer afucosylated monovalent antibody construct comprising transfecting the at least one mammalian cell with a multi-cistronic vector comprising at least two of said first, second and third DNA sequence. In an embodiment, said at least one mammalian cell is selected from the group consisting of a VERO, HeLa, HEK, NSO, Chinese Hamster Ovary (CHO), W138, BHK, COS-7, Caco-2 and MDCK cell, and subclasses and variants thereof.

In an embodiment is provided the method of producing a glycosylated monovalent antibody construct or a or glycoengineer afucosylated monovalent antibody construct, wherein said predetermined ratio of the first DNA sequence:second DNA sequence:third DNA sequence is about 1:1:1.

In another embodiment is the method of producing a glycosylated monovalent antibody construct or a or glycoengineer afucosylated monovalent antibody construct described herein, wherein said predetermined ratio of the first DNA sequence:second DNA sequence:third DNA sequence is such that the amount of translated first heavy chain polypeptide is about equal to the amount of the second heavy chain polypeptide, and the amount of the light chain polypeptide. In an embodiment is the method described herein wherein the expression product of the at least one stable mammalian cell comprises a larger percentage of the desired glycosylated monovalent antibody as compared to the monomeric heavy or light chain polypeptides, or other antibodies.

In an embodiment is provided the method of producing a glycosylated monovalent antibody construct or a or glycoengineer afucosylated monovalent antibody construct described herein, comprising identifying and purifying the desired glycosylated monovalent antibody. In certain embodiments, said identification is by one or both of liquid chromatography and mass spectrometry.

Provided herein is a method of producing antibody constructs with improved ADCC comprising: transfecting at least one stable mammalian cell with: a first DNA sequence encoding a first heavy chain polypeptide comprising a heavy chain variable domain and a first Fc domain polypeptide; a second DNA sequence encoding a second heavy chain polypeptide comprising a second Fc domain polypeptide, wherein said second heavy chain polypeptide is devoid of a variable domain; and a third DNA sequence encoding a light chain polypeptide comprising a light chain variable domain, such that the said first DNA sequence, said second DNA sequence and said third DNA sequences are transfected in said mammalian cell in a pre-determined ratio; translating the said first DNA sequence, said second DNA sequence, and said third DNA sequence in the at least one mammalian cell such that said heavy and light chain polypeptides are expressed as a glycosylated monovalent antibody in said at least one stable mammalian cell, wherein said glycosylated monovalent asymmetric antibody has a higher ADCC as compared to a corresponding wild-type antibody.

Provided herein is a method of producing HER2 binding antibody constructs with at least one of improved ADCC, ADCP and CDC, comprising: transfecting at least one stable mammalian cell with: a first DNA sequence encoding a first heavy chain polypeptide comprising a heavy chain variable domain and a first Fc domain polypeptide; a second DNA sequence encoding a second heavy chain polypeptide comprising a second Fc domain polypeptide, wherein said second heavy chain polypeptide is devoid of a variable domain; and a third DNA sequence encoding a light chain polypeptide comprising a light chain variable domain, such that the said first DNA sequence, said second DNA sequence and said third DNA sequences are transfected in said mammalian cell in a pre-determined ratio; translating the said first DNA sequence, said second DNA sequence, and said third DNA sequence in the at least one mammalian cell such that said heavy and light chain polypeptides are expressed as an asymmetric glycosylated monovalent HER2 binding antibody in said at least one stable mammalian cell, wherein said glycosylated monovalent HER2 binding antibody has at least one of improved ADCC, ADCP and CDC as compared to a corresponding wild-type HER2 binding antibody.

Provided is a method of increasing antibody concentration on at least one target cell providing to the target cell a monovalent antibody construct comprising: an antigen-binding polypeptide construct which monovalently binds an antigen; a dimeric Fc region; wherein said monovalent antibody construct displays an increase in binding density and Bmax to a target cell displaying said antigen as compared to a corresponding bivalent antibody construct with two antigen binding regions, and wherein said monovalent antibody construct shows improved efficacy compared to a corresponding bivalent antibody construct, and wherein said improved efficacy is not caused by crosslinking of the antigen, antigen dimerization, prevention of antigen modulation, antigen internalization or antigen downregulation, or antigen activation.

Provided herein is a pharmaceutical composition comprising a monovalent antibody construct described herein and a pharmaceutically acceptable carrier. In certain embodiments is a pharmaceutical composition described herein, further comprising a drug molecule conjugated to the monovalent antibody construct.

Provided herein is a method of treating cancer comprising providing to a patient in need thereof an effective amount of the pharmaceutical composition described herein. Provided is a method of treating disorder of HER signaling providing to a patient in need thereof an effective amount of the pharmaceutical composition of described herein. Provided herein is a method of inhibiting growth of a tumor, comprising contacting the tumor with a composition comprising an effective amount of the monovalent antibody construct described herein. Provided is a method of shrinking a tumor, comprising contacting the tumor with a composition comprising an effective amount of the monovalent antibody construct described herein.

Provided is a method of treating breast cancer comprising, providing to a patient in need thereof an effective amount of a monovalent antibody construct described herein. In an embodiment is a method of treating breast cancer in a patient partially responsive to treatment with one or more of Trastuzumab, pertuzumab, TDM1 and anti-HER bivalent antibodies, said method comprising providing to a patient in need thereof an effective amount of a monovalent antibody construct described herein. In an embodiment is a method of treating breast cancer in a patient not responsive to treatment with one or more of Trastuzumab, pertuzumab, TDM1 (ADC) and anti-HER bivalent antibodies, comprising providing to a patient in need thereof an effective amount of a monovalent antibody construct described herein. Provided is a method of treating breast cancer described herein wherein said method comprises providing said antibody construct in addition to another therapeutic agent. In an embodiment is a method of treating breast cancer described herein, wherein said antibody construct is provided simultaneously with said therapeutic agent. Also provided is a method of treating breast cancer provided herein, wherein said antibody construct is conjugated with said therapeutic agent.

Provided is the isolated monovalent antibody construct described herein wherein the monovalent antibody construct is conjugated to one or more drug molecules.

Provided is a method of inhibiting multimerization of an antigen molecule, comprising contacting the antigen with a composition comprising an effective amount of the monovalent antibody construct described herein. Also provided is a method of inhibiting binding of an antigen to its cognate binding partner comprising contacting the antigen with a composition comprising an amount of the monovalent antibody construct described herein, sufficient to bind to the antigen.

Also provided are transgenic organisms modified to contain nucleic acid molecules described herein to encode and express monovalent antibody constructs described herein.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

In drawings which illustrate embodiments of the invention,

FIG. 1 depicts an illustration of antibody Fc dependent cytotoxicity namely complement-dependent cytotoxicity (CDC), antibody-dependent cellular cytotoxicity (ADCC), and antibody dependent cellular phagocytosis (ADCP).

FIG. 2A depicts monovalent antibodies binding to antigen. FIG. 2A depicts a monovalent antibody construct described herein that binds antigen with a 1:1 stochiometry. As described herein, the monovalent antibody constructs result in a higher antibody concentration/decoration on a per cell basis and result in greater Fc mediated cell killing by ADCC, CDC, ADCP.

FIG. 2B depicts bivalent antibodies binding to antigen. FIG. 2B depicts a bivalent antibody construct that binds antigen with a 1:2 stochiometry.

FIG. 3A depicts the ability of an exemplary monovalent anti-HER2 antibody to bind to SKOV3 cells as a non-linear fit binding curve; B. log transformed curve.

FIG. 3B depicts the ability of an exemplary monovalent anti-HER2 antibody to bind to SKOV3 cells as a log transformed curve.

FIG. 4A depicts the ability of exemplary monovalent anti-HER2 antibodies to bind to cell expressing HER2 in varying density: MDA-MB-231 cells.

FIG. 4B depicts the ability of exemplary monovalent anti-HER2 antibodies to bind to cell expressing HER2 in varying density: SKOV3 cells.

FIG. 4C depicts the ability of exemplary monovalent anti-HER2 antibodies to bind to cell expressing HER2 in varying density: SKBR3 cells.

FIG. 5 depicts the ability of an exemplary monovalent anti-HER2 antibody to mediate enhanced ADCC compared to a bivalent, full-sized antibody (FSA).

FIG. 6 depicts the ability of an exemplary monovalent anti-HER2 antibody to mediate enhanced CDC compared to a bivalent, full-sized antibody (FSA).

FIG. 7A depicts the ability of an exemplary monovalent anti-HER2 antibody to mediate enhanced CDC compared to a bivalent, full-sized antibody (FSA): FIG. 7A represents an experiment in which two PBMC donors were used.

FIG. 7B depicts the ability of an exemplary monovalent anti-HER2 antibody to mediate enhanced CDC compared to a bivalent, full-sized antibody (FSA): FIG. 7B represents an experiment in which two PBMC donors were used.

FIG. 7C depicts a summary of two separate experiments with OA2-Fab-HER2 and 4 PBMC donors, with the percent CD16+ cell indicated per donor. Data is normalized to the maximum lysis of WT FSA Hcptn, and the fold difference in maximum lysis of OA2-Fab-HER2 vs WT FSA Hcptn is presented.

FIG. 8A depicts the analysis of yield and purity of exemplary monovalent anti-HER2 antibodies post protein A purification. FIG. 8A is SDS-PAGE analysis of purified monovalent anti-HER2 antibodies.

FIG. 8B depicts the analysis of yield and purity of exemplary monovalent anti-HER2 antibodies post protein A purification. FIG. 8B is LCMS analysis of OA1-Fab-HER2.

FIG. 8C depicts the analysis of yield and purity of exemplary monovalent anti-HER2 antibodies post protein A purification. FIG. 8C is LCMS analysis of OA2-Fab-HER2.

FIG. 8D depicts the analysis of yield and purity of exemplary monovalent anti-HER2 antibodies post protein A purification. FIG. 8D is an expanded view of the LCMS spectrum for OA2-Fab-HER2 showing the lower mass peptides at ˜0.8% Two Light chains+1 Short Heavy chain (72,898 Da), ˜0.7% Short Heavy chain alone (25,907 Da).

FIG. 9A depicts the ability of monovalent anti-HER2 antibodies to be internalized. FIG. 9A. Results plotted as % internalization.

FIG. 9B depicts the ability of monovalent anti-HER2 antibodies to be internalized. FIG. 9B. Results plotted as % effect relative to control.

FIG. 10 depicts the ability of monovalent anti-HER2 antibodies to inhibit growth of SKBR3 cells.

FIG. 11A depicts the ability of monovalent anti-HER2 antibodies to bind to FcRn receptors.

FIG. 11B depicts the ability of monovalent anti-HER2 antibodies to bind to FcRn receptors.

FIG. 11C depicts the ability of monovalent anti-HER2 antibodies to bind to FcRn receptors.

FIG. 12 depicts the ability of another exemplary monovalent anti-HER2 antibody to bind to SKOV3 cells.

FIG. 13A depicts the DNA and amino acid sequences of FSA-scFv-HER2. FIG. 13A. (SEQ ID NO: 3) DNA sequence of chain A.

FIG. 13B depicts the DNA and amino acid sequences of FSA-scFv-HER2. FIG. 13B. (SEQ ID NO: 4) amino acid sequence of chain A.

FIG. 13C depicts the DNA and amino acid sequences of FSA-scFv-HER2. FIG. 13C. (SEQ ID NO: 5) DNA sequence of chain B.

FIG. 13D depicts the DNA and amino acid sequences of FSA-scFv-HER2. FIG. 13D. (SEQ ID NO: 6) amino acid sequence chain B.

FIG. 14A depicts the DNA sequence of OA3-scFv-HER2 chain A. (SEQ ID NO: 7).

FIG. 14B depicts the amino acid sequence of OA3-scFv-HER2 chain A (SEQ ID NO: 8).

FIG. 14C depicts the DNA sequence of OA3-scFv-HER2 chain B (SEQ ID NO: 9).

FIG. 14D depicts the amino acid sequence of OA3-scFv-HER2 chain B (SEQ ID NO: 10).

FIG. 15A depicts the DNA heavy chain A sequence of OA1-Fab-HER2 (SEQ ID NO: 11).

FIG. 15B depicts the amino acid heavy chain A sequence of OA1-Fab-HER2 (SEQ ID NO: 12).

FIG. 15C depicts the DNA light chain sequence of OA1-Fab-HER2 (SEQ ID NO: 13).

FIG. 15D depicts the amino acid light chain sequence of OA1-Fab-HER2 (SEQ ID NO: 14).

FIG. 15E depicts the DNA heavy chain B sequence of OA1-Fab-HER2 (SEQ ID NO: 15).

FIG. 15F depicts the amino acid heavy chain B sequence of OA1-Fab-HER2 (SEQ ID NO: 16).

FIG. 16A depicts the DNA heavy chain A sequence of OA2-Fab-HER2 (SEQ ID NO: 17).

FIG. 16B depicts the amino acid heavy chain A sequence of OA2-Fab-HER2 (SEQ ID NO: 18).

FIG. 16C depicts the DNA light chain sequence of OA2-Fab-HER2 (SEQ ID NO: 19).

FIG. 16D depicts the amino acid light chain sequence of OA2-Fab-HER2 (SEQ ID NO: 20).

FIG. 16E depicts the DNA heavy chain B sequence of OA2-Fab-HER2 (SEQ ID NO: 21).

FIG. 16F depicts the amino acid heavy chain B sequence of OA2-Fab-HER2 (SEQ ID NO: 22).

FIG. 17A depicts the DNA heavy chain A sequence of wt FSA Hcptn (SEQ ID NO: 23).

FIG. 17B depicts the amino acid heavy chain A sequence of wt FSA Hcptn (SEQ ID NO: 24).

FIG. 17C depicts the DNA light chain sequence of wt FSA Hcptn (SEQ ID NO: 25).

FIG. 17D depicts the amino acid light chain sequence of wt FSA Hcptn (SEQ ID NO: 26).

FIG. 18A depicts the DNA heavy chain A sequence of FSA-Fab-HER2 (SEQ ID NO: 27).

FIG. 18B depicts the amino acid heavy chain A sequence of FSA-Fab-HER2 (SEQ ID NO: 28).

FIG. 18C depicts the DNA light chain sequence of FSA-Fab-HER2 (SEQ ID NO: 29).

FIG. 18D depicts the amino acid light chain sequence of FSA-Fab-HER2 (SEQ ID NO: 30).

FIG. 18E depicts the DNA heavy chain B sequence of FSA-Fab-HER2 (SEQ ID NO: 31).

FIG. 18F depicts the amino acid heavy chain B sequence of FSA-Fab-HER2 (SEQ ID NO: 32).

FIG. 19A depicts the DNA and amino acid sequences of FSA-scFv-BID2. (SEQ ID NO: 33) DNA sequence of chain A and chain B.

FIG. 19B depicts the DNA and amino acid sequences of FSA-scFv-BID2. (SEQ ID NO: 34) amino acid sequence of chain A and chain B.

FIG. 20A depicts the DNA chain A sequence of OA4-scFv-BID2 (SEQ ID NO: 35).

FIG. 20B depicts the amino acid chain A sequence of OA4-scFv-BID2 (SEQ ID NO: 36).

FIG. 20C depicts the DNA chain B sequence of OA4-scFv-BID2 (SEQ ID NO: 37).

FIG. 20D depicts the amino acid chain B sequence of OA4-scFv-BID2 (SEQ ID NO: 38).

FIG. 21A depicts the ability of exemplary monovalent antibody constructs to mediate ADCC in different cell lines. FIG. 21A depicts the results in MCF7 cells.

FIG. 21B depicts the ability of exemplary monovalent antibody constructs to mediate ADCC in different cell lines. FIG. 21B depicts the results in MDA-MB-231 cells.

FIG. 21C depicts the ability of exemplary monovalent antibody constructs to mediate ADCC in different cell lines. FIG. 21C depicts the results in MCF7 cells.

FIG. 21D depicts the ability of exemplary monovalent antibody constructs to mediate ADCC in different cell lines. FIG. 21D depicts the results in MCF7 cells.

FIG. 21E depicts the ability of exemplary monovalent antibody constructs to mediate ADCC in different cell lines. FIG. 21E depicts the results in MCF7 cells.

FIG. 22 depicts the pharmacokinetic profile of an exemplary monovalent antibody construct in mice.

FIG. 23A depicts the effect of treatment of SKBr3 cells with an exemplary monovalent anti-Her2 antibody (OA1-Fab-Her2) on phosphorylation of signaling molecules. The panel shows the effect on phosphorylation of ErbB2.

FIG. 23B depicts the effect of treatment of SKBr3 cells with an exemplary monovalent anti-Her2 antibody (OA1-Fab-Her2) on phosphorylation of signaling molecules. The panel shows the effect on phosphorylation of MAPK and AKT.

FIG. 24A shows the quantitative assessment of the degree of phosphorylation of Akt as measured by ELISA at 15 minute.

FIG. 24B shows the quantitative assessment of the degree of phosphorylation of Akt as measured by ELISA at 30 minutes.

FIG. 25A depicts the ability of exemplary monovalent antibody constructs according to the invention to bind to JIMT-1 cells.

FIG. 25B depicts the ability of exemplary monovalent antibody constructs according to the invention to bind to BT474 cells.

FIG. 25C depicts the ability of exemplary monovalent antibody constructs according to the invention to bind to MCF-7 cells.

FIG. 26A depicts the ability of exemplary monovalent anti-Her2 antibodies to inhibit the growth of BT-474 cells (OA1-Fab-Her2 OA2-Fab-HER2).

FIG. 26B depicts the ability of exemplary monovalent anti-Her2 antibodies to inhibit the growth of BT-474 cells (OA5-Fab-HER2, OA6-Fab-Her2).

FIG. 27A depicts the ability of the exemplary monovalent antibody constructs OA1-Fab-Her2 and OA5-Fab-Her2 (at 200 nM) to internalize in BT-474 cells.

FIG. 27B depicts the ability of the exemplary monovalent antibody constructs OA1-Fab-Her2 and OA5-Fab-Her2 (at 200 nM) to internalize in JIMT-1 cells.

FIG. 28 depicts the ability of an exemplary monovalent antibody construct to bind to MALME-3M cells.

FIG. 29 depicts the ability of an exemplary monovalent antibody construct-antibody drug conjugate (ADC) to kill BT474 cells.

FIG. 30A depict the purity of constructs. FIG. 30A depicts purity of the exemplary monovalent antibody constructs OA5-Fab-Her2 and OA6-Fab-Her2 post protein A purification.

FIG. 30B depict the purity of constructs. FIG. 30 B shows heterodimer purity analysis by LC/MS which indicates that both OA5-Fab-Her2 and OA6-Fab-Her2 can be purified to greater than 99% purity post protein A and size exclusion chromatography.

FIG. 31A depicts the DNA sequence of OA5-Fab-HER2 chain A (SEQ ID NO:39).

FIG. 31B depicts the amino acid sequence of OA5-Fab-HER2 chain A (SEQ ID NO:40).

FIG. 31C depicts the DNA sequence of OA5-Fab-HER2 chain B (SEQ ID NO:41).

FIG. 31D depicts the amino acid sequence of OA5-Fab-HER2 chain B (SEQ ID NO:42).

FIG. 31E depicts the DNA sequence of OA5-Fab-HER2 light chain (SEQ ID NO:43).

FIG. 31F depicts the amino acid sequence of OA5-Fab-HER2 light chain (SEQ ID NO:44).

FIG. 32A depicts the DNA sequence of OA6-Fab-HER2 chain A (SEQ ID NO:45).

FIG. 32B depicts the amino acid sequence of OA6-Fab-HER2 chain A (SEQ ID NO:46).

FIG. 32C depicts the DNA sequence of OA6-Fab-HER2 chain B (SEQ ID NO:47).

FIG. 32D depicts the amino acid sequence of OA6-Fab-HER2 chain B (SEQ ID NO:48).

FIG. 32E depicts the DNA sequence of OA6-Fab-HER2 light chain (SEQ ID NO:49).

FIG. 32F depicts the amino acid sequence of OA6-Fab-HER2 light chain (SEQ ID NO:50).

FIG. 33A depicts the DNA sequence of FSA-Fab-pert chain A (SEQ ID NO: 51).

FIG. 33B depicts the amino acid sequence of FSA-Fab-pert chain A (SEQ ID NO: 52).

FIG. 33C depicts the DNA sequence of FSA-Fab-pert chain B (SEQ ID NO: 53).

FIG. 33D depicts the amino acid sequence of FSA-Fab-pert chain B (SEQ ID NO: 54).

FIG. 33E depicts the DNA sequence of FSA-Fab-pert light chain (SEQ ID NO: 55).

FIG. 33F depicts the amino acid sequence of FSA-Fab-pert light chain (SEQ ID NO: 56).

### DETAILED DESCRIPTION

Provided herein are monovalent antibody constructs comprising an antigen-binding polypeptide construct which monovalently binds an antigen; and a dimeric Fc polypeptide construct comprising two monomeric Fc polypeptides each comprising a CH3 domain, wherein one said monomeric Fc polypeptide is fused to at least one polypeptide from the antigen-binding polypeptide construct; wherein said monovalent antibody construct displays an increase in binding density and to a target cell displaying said antigen as compared to a corresponding monospecific bivalent antibody construct with two antigen binding regions, and wherein said monovalent antibody construct shows superior efficacy and/or bioactivity as compared to the corresponding bivalent antibody construct, and wherein said superior efficacy and/or bioactivity is the result of the increase in binding density and resulting increase in decoration of a target cell. The increase in Bₘₐₓ or binding density and resultant increase in target decoration by the monovalent antibody construct provided here is the effect of specific target binding and not due to nonspecific binding. In certain embodiments the maximum binding occurs at a target to antibody ratio of 1:1.

In certain embodiments, the monovalent antibody constructs provided herein possess at least one or more of the following attributes: increased compared to corresponding monospecific bivalent antibody constructs (FSA); K_{d} comparable to corresponding FSA; same or slower off-rate compared to corresponding FSA; decreased or partial agonism; no cross-linking and dimerization of targets; specificity and/or selectivity for target cell of interest; full or partial or no inhibition of target cell growth; complete Fc capable of inducing effector activity; and ability to be internalized by target cell.

In certain embodiments, the monovalent antibody constructs provided herein possess the following minimal attributes: increased Bₘₐₓ compared to corresponding FSA; K_{d} comparable to corresponding FSA; same or slower off-rate compared to corresponding FSA; decreased or partial agonism; no cross-linking and dimerization of targets; specificity and/or selectivity for target cell of interest; full or partial or no inhibition of target cell growth; complete Fc capable of inducing effector activity; and optionally ability to be internalized by target cell.

Provided herein is a monovalent antibody construct wherein said construct is at least one of: a monovalent lytic antibody, a monovalent internalizing antibody and combinations thereof. In some embodiments, the antibody construct is a monovalent lytic antibody and/or a monovalent internalizing antibody depending on the balance these antibodies display between the following efficacy factors: a) the ability of the monovalent antibody construct to be internalized, b) the increased Bₘₐₓ and Kd/on-off rate of the monovalent antibody construct, and c) the degree of agonism/partial agonism of the monovalent antibody construct

Provided herein is a method of increasing antibody concentration in at least one target cell comprising providing to the target cell a monovalent antibody construct comprising: an antigen-binding polypeptide construct which monovalently binds an antigen; a dimeric Fc region; wherein said monovalent antibody construct displays an increase in binding density and Bmax (maximum binding) to a target cell displaying said antigen as compared to a corresponding bivalent antibody construct with two antigen binding regions, and wherein said monovalent antibody construct shows better therapeutic efficacy compared to a corresponding bivalent antibody construct, and wherein said efficacy is not caused by crosslinking of the antigen, antigen dimerization, prevention of antigen modulation, or prevention of antigen activation. Conversely, the other is true that efficacy can be caused by antigen modulation or antigen activation so long as these do not overcome the net killing effect.

In some embodiments is an isolated monovalent antibody construct described herein, wherein said antibody construct exhibits no avidity.

Monovalent Lytic (Mv-L) Antibodies

Provided are monovalent antibody constructs described herein wherein said constructs possess an increased Bₘₐₓ and comparable or slower off rate as compared to FSA (thus resulting in higher decoration of the target cell with the MV-L and antibody dependent cytotoxicity). In some embodiments, MV-L antibody constructs described herein bind the target cell with increased Bₘₐₓ and fast on and slow off rate compared to FSA. In some embodiments, MV-L antibody constructs described herein block cognate ligand binding to the target antigen. In some embodiments, MV-L antibody constructs described herein show no internalization thereby resulting in the maximal decoration of antibody on a cell and functional blockade of the pathway.

In certain embodiments MV-L antibody constructs 1) bind and saturate the target cell with increased Bₘₐₓ and fast on and a similar or slower off rate compared to FSA; 2) are non-agonistic; 3) inhibit cell growth; 4) block cognate ligand binding to the target antigen; 5) show no internalization and 6) comprise an Fc domain that engages in effector activity. In certain embodiments, MV-L antibody constructs maximally decorate the target cell surface, and block activation of the target cell by the target antigen without causing counteracting activities that can result in cell survival and growth.

In one embodiment, the monovalent lytic antibody constructs according to the invention 1) bind the target cell with increased Bₘₐₓ and have a fast on-rate and similar or slow-off rate compared to monospecific bivalent antibody constructs, 2) are non-agonistic; 3) inhibit cell growth, 4) block cognate ligand binding to the target antigen, 5) show minimal internalization and 6) comprise an Fc domain that interacts with the Fc receptors and the complement system to engage the immune system.

In certain embodiments MV-L antibody constructs are capable of binding to FcγR receptors and complement proteins and at high cell surface concentrations are more effective at inducing antibody dependent cytotoxicity. In certain embodiments is an MV-L antibody construct useful to kill target cells through Fc effector functions such as ADCC, ADCP or CDC.

In one embodiment, the MV-L antibody construct is able to preferentially engage the effector system as a result of steric differences relative to the engagement achieved by FSA. In certain embodiments, MV-L substantially block ligand binding to the target antigen while showing no agonism, however increased Bmax and fast on-rate plus similar or slow off-rate as compared to the FSA can overcome partial blockade of ligand, some degree of agonism and cell growth, and internalization to result in a net efficacious effect that is still superior to FSAs. In some embodiments, the MV-L antibody construct provided herein binds HER2. In some embodiments, the antibody construct binds at least one HER2 extracellular domain. In certain embodiments, the extracellular domain is at least one of ECD1, ECD2, ECD3 and ECD4. In certain embodiments the HER2 binding MV-L is OA5-Fab-Her2 (4182) or OA1-Fab-Her2 (1040) provided herein.

In certain embodiments increased decoration of diseased cells with a monovalent lytic antibody construct (MV-L) results in target cell depletion via ADCC, CDC or ADCP more effectively than a monospecific bivalent antibody construct (FSA).

Monovalent Internalizing (MV-Int) Antibodies

Provided herein are monovalent antibody constructs comprising an antigen-binding polypeptide construct which monovalently binds an antigen; and a dimeric Fc polypeptide construct comprising two monomeric Fc polypeptides each comprising a CH3 domain, and wherein said monovalent antibody constructs are monovalent internalizing (MV-Int) antibody constructs. In certain embodiments the increased Bₘₐₓ and the degree of internalization are the key drivers for classifying monovalent antibody constructs in the MV-Int category. In certain embodiments MV-Int antibody constructs bind the target cell with increased Bₘₐₓ and fast on-plus similar or slow off-rate compared to FSA. In some embodiments the My-Int causes at least one of: higher decoration of the target cell, blocking cognate ligand binding to the target antigen and effectively internalizing, and inhibition or no induction of any cell growth. In some embodiments, the MV-L antibody provided herein binds HER2. In certain embodiments the HER2 binding MV-Int is OA5-Fab-Her2 (4182) or OA1-Fab-Her2 (1040) provided herein.

In certain embodiments provided herein are MV-Int constructs which have a high Bmax and high internalization as compared to MV-L and FSAs, thereby resulting in higher intracellular concentrations of MV-Int. In some embodiments, the MV-L antibody provided herein binds HER2. In some embodiments, the antibody construct binds at least one HER2 extracellular domain. In certain embodiments, the extracellular domain is at least one of ECD1, ECD2, ECD3 and ECD4. In some embodiments, the MV-L antibody inhibits dimerization of HER2 extracellular domains. In some embodiments, the antibody construct binds at least one HER2 extracellular domain. In certain embodiments, the extracellular domain is at least one of ECD1, ECD2, ECD3 and ECD4.

In some embodiments, the MV-Int antibodies can partially activate a receptor using it as a Trojan to shuttle the antibody construct described herein, optionally with a payload into a cell. Such MV-Int antibodies are suitable for use in the preparation of antibody-drug conjugates (ADCs) and can be used in the treatment of indications where delivery of a toxic drug to the target cell is desired. With this modality, the delivery of a highly toxic payload resulting in acute cell death would overcome some agonistic activity conferred in the MV-Int. In some embodiments, the MV-Int antibody provided herein binds HER2. In certain embodiments are HER2 binding monovalent antibody constructs that are both MV-L and MV-Int. For instance OA1-Fab-Her2 (1040)-v1040 exhibits sufficient properties for a MV-L and MV-Int.

In one embodiment, the higher decoration and Bmax achieved by the MV-Int relative to the FSA could compensate for the difference in level of internalization.

In one embodiment, the My-Int antibody constructs 1) bind the target cell with increased Bₘₐₓ and fast on-rate plus comparable or slow off-rate compared to FSA (thus resulting in higher decoration of the target cell with the MV-Int), 2) block cognate ligand binding to the target antigen; 3) are non-agonistic; 4) do not induce cell growth, and 5) are effectively internalized to a greater degree than monospecific bivalent antibody constructs. In another embodiment, the monovalent internalizing antibody constructs 1) bind the target cell with increased Bₘₐₓ and fast on-rate plus slow off-rate compared to FSA (thus resulting in higher decoration of the target cell with the MV-Int), 2) block cognate ligand binding to the target antigen; 3) are only partially-agonistic; 4) do not induce cell growth, and 5) are effectively internalized to a greater degree than monospecific bivalent antibody constructs.

In some embodiments increased decoration and internalization of monovalent internalizing antibody constructs (MV-Int) by immune T and B cells and diseased cells and drug resistant diseased cells results in target cell depletion via ADC more effectively than FSA. In one embodiment, monovalent internalizing antibody constructs (MV-Int) conjugated to a drug molecule are useful in the treatment of drug refractory and resistant patients, and patients who fail to respond to first-line therapies. In some embodiments, the MV-Int antibody provided herein binds HER2. In certain embodiments are HER2 binding monovalent antibody constructs that are both MV-L and MV-Int. For instance OA1-Fab-Her2 (1040)

In an embodiment, the increased decoration of pathogens such as viruses with a monovalent lytic antibody construct (MV-L) described herein results in pathogen depletion more effectively than a monospecific bivalent antibody construct (FSA). For example, viruses such as HIV have evolved to evade bivalent antibodies and bivalent binding by having low density of envelope spikes, a distinguishing feature when compared with viruses to which protective neutralizing antibody responses are consistently raised. The result is a minimization of avidity, normally used by antibodies to achieve high affinity binding and potent neutralization, thereby allowing viruses to evade antibodies. Monovalent antibody constructs described herein are not impacted as significantly since binding is to a single epitope. In certain embodiments, monovalent antibody constructs described herein can be used alone or as a combination to blanket all distinct viral epitopes.

In certain embodiments, MV_L antibody constructs described herein are used for direct targeting and antibody mediated clearance via opsonization of pathogens. In certain embodiments, MV-L and MV-Int antibodies are both suitable for antibody-dependent deletion of pathogen infected cells. In some embodiments, MV-L and MV-Int antibody constructs highly decorate HIV-infected T cells and mark these cells for depletion by ADCC, CDC, ADCP or ADC killing. In certain embodiments, monovalent antibody constructs described herein can be used alone or in combination with other monovalent antibody constructs.

Provided herein is an isolated monovalent antibody construct comprising an antigen-binding polypeptide construct which monovalently binds an antigen; and a dimeric Fc polypeptide construct comprising two monomeric Fc polypeptides each comprising a CH3 domain, wherein one said monomeric Fc polypeptide is fused to at least one polypeptide from the antigen-binding polypeptide construct; wherein said monovalent antibody construct displays an increase in binding density and Bmax (maximum binding) to a target cell displaying said antigen as compared to a corresponding FSA construct with two antigen binding regions, wherein said monovalent antibody construct shows superior efficacy and/or bioactivity as compared to the corresponding bivalent antibody construct, and wherein said superior efficacy and/or bioactivity is the result of the increase in binding density.

Provided herein is an isolated monovalent antibody construct comprising an antigen-binding polypeptide construct which monovalently binds an antigen; and a dimeric Fc polypeptide construct comprising two monomeric Fc polypeptides each comprising a CH3 domain, wherein one said monomeric Fc polypeptide is fused to at least one polypeptide from the antigen-binding polypeptide construct; wherein said monovalent antibody construct displays an increase in binding density and Bmax (maximum binding) to a target cell displaying said antigen as compared to a corresponding FSA construct with two antigen binding regions, wherein said monovalent antibody construct shows superior efficacy and/or bioactivity as compared to the corresponding bivalent antibody construct, and wherein said superior efficacy and/or bioactivity is the result of the increase in binding density.

Provided in certain embodiments is an isolated monovalent antibody construct described herein, wherein the increase in binding density and Bmax relative to a monospecific bivalent antibody is observed at a concentration greater than the observed equilibrium constant (Kd) and at saturating concentrations of the antibodies. In some embodiments the superior efficacy and/or bioactivity is the result of increased FcγR or complement (C1q) binding and at least one of higher ADCC, higher ADCP and higher CDC as compared to said corresponding bivalent antibody construct. In specific embodiments, the isolated monovalent antibody construct is anti-proliferative and is internalized. In certain embodiments is an isolated monovalent antibody construct described herein wherein said increase in binding density and Bmax relative to the FSA is independent of the density of the antigen on the target cell. In some embodiments is provided an isolated monovalent antibody construct described herein, wherein the target cell is a cancer cell, or a HER2 expressing diseased cell. In an embodiment, the isolated monovalent antibody construct described herein exhibits no avidity.

### DEFINITIONS

It is to be understood that this invention is not limited to the particular protocols; cell lines, constructs, and reagents described herein and as such may vary. It is also to be understood that the terminology used herein is for the purpose of describing particular embodiments only, and is not intended to limit the scope of the present invention, which will be limited only by the appended claims.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are incorporated herein by reference for the purpose of describing and disclosing, for example, the constructs and methodologies that are described in the publications, which might be used in connection with the presently described invention. The publications discussed herein are provided solely for their disclosure prior to the filing date of the present application. Nothing herein is to be construed as an admission that the inventors are not entitled to antedate such disclosure by virtue of prior invention or for any other reason.

A “dimer” or “heterodimer” is a molecule comprising at least a first monomer polypeptide and a second monomer polypeptide. In the case of a heterodimer, one of said monomers differs from the other monomer by at least one amino acid residue. In certain embodiments, the assembly of the dimer is driven by surface area burial. In some embodiments, the monomeric polypeptides interact with each other by means of electrostatic interactions and/or salt-bridge interactions that drive dimer formation by favoring the desired dimer formation and/or disfavoring formation of other non-desired specimen. In some embodiments, the monomer polypeptides interact with each other by means of hydrophobic interactions that drive desired dimer formation by favoring desired dimer formation and/or disfavoring formation of other assembly types. In certain embodiments, the monomer polypeptides interact with each other by means of covalent bond formation. In certain embodiments, the covalent bonds are formed between naturally present or introduced cysteines that drive desired dimer formation. In certain embodiments described herein, no covalent bonds are formed between the monomers. In some embodiments, the polypeptides interact with each other by means of packing/size-complementarity/knobs-into-holes/protuberance-cavity type interactions that drive dimer formation by favoring desired dimer formation and/or disfavoring formation of other non-desired embodiments. In some embodiments, the polypeptides interact with each other by means of cation-pi interactions that drive dimer formation. In certain embodiments the individual monomer polypeptides cannot exist as isolated monomers in solution.

The term “Fc region”, as used herein, generally refers to a dimer complex comprising the C-terminal polypeptide sequences of an immunoglobulin heavy chain, wherein a C-terminal polypeptide sequence is that which is obtainable by papain digestion of an intact antibody. The Fc region may comprise native or variant Fc sequences. Although the boundaries of the Fc sequence of an immunoglobulin heavy chain might vary, the human IgG heavy chain Fc sequence is usually defined to stretch from an amino acid residue at about position Cys226, or from about position Pro230, to the carboxyl terminus of the Fc sequence. The Fc sequence of an immunoglobulin generally comprises two constant domains, a CH2 domain and a CH3 domain, and optionally comprises a CH4 domain. By “Fc polypeptide” herein is meant one of the polypeptides that make up an Fc region. An Fc polypeptide may be obtained from any suitable immunoglobulin, such as IgG1, IgG2, IgG3, or IgG4 subtypes, IgA, IgE, IgD or IgM. In some embodiments, an Fc polypeptide comprises part or all of a wild type hinge sequence (generally at its N terminus). In some embodiments, an Fc polypeptide does not comprise a functional or wild type hinge sequence.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell.

“Complement dependent cytotoxicity” and “CDC” refer to the lysing of a target in the presence of complement. The complement activation pathway is initiated by the binding of the first component of the complement system (C1q) to a molecule (e.g. an antibody) complexed with a cognate antigen.

“Antibody-dependent cellular phagocytosis and “ADCP” refer to the destruction of target cells via monocyte or macrophage-mediated phagocytosis.

The terms “Fc receptor” and “FcR” are used to describe a receptor that binds to the Fc region of an antibody. For example, an FcR can be a native sequence human FcR. Generally, an FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Immunoglobulins of other isotypes can also be bound by certain FcRs (see, e.g., Janeway et al., Immuno Biology: the immune system in health and disease, (Elsevier Science Ltd., NY) (4th ed., 1999)). Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976); and Kim et al., J. Immunol. 24:249 (1994)).

A “disorder” is any condition that would benefit from treatment with an antibody or method of the invention. This includes chronic and acute disorders or diseases including those pathological conditions which predispose the mammal to the disorder in question. Non-limiting examples of disorders to be treated herein include malignant and benign tumors; non-leukemias and lymphoid malignancies; neuronal, glial, astrocytal, hypothalamic and other glandular, macrophagal, epithelial, stromal and blastocoelic disorders; and inflammatory, immunologic and other angiogenesis-related disorders.

The terms “cancer” and “cancerous” refer to or describe the physiological condition in mammals that is typically characterized by unregulated cell growth/proliferation. Examples of cancer include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, myeloma (e.g., multiple myeloma), hepatocellular cancer, gastrointestinal cancer, pancreatic cancer, glioblastoma/glioma (e.g., anaplastic astrocytoma, glioblastoma multiforme, anaplastic oligodendroglioma, anaplastic oligodendroastrocytoma), cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

As used herein the term “inflammatory disease(s)” or “inflammatory disorder(s) encompass conditions characterized by inflammation in the connective tissues, or degeneration of these tissues. In certain embodiments, the inflammatory disease or disorder includes but is not restricted to Alzheimer's, ankylosing spondylitis, arthritis including but not restricted to osteoarthritis, rheumatoid arthritis (RA) and psoriatic arthritis, asthma, atherosclerosis, Crohn's disease, colitis, dermatitis, diverticulitis, fibromyalgia, hepatitis, irritable bowel syndrome (IBS), systemic lupus erythematous (SLE), nephritis, Parkinson's disease and ulcerative colitis.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishing of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or disorder. In one embodiment, antibodies and methods of the invention effect tumor regression. In one embodiment, antibodies and methods of the invention effect inhibition of tumor/cancer growth.

The term “substantially purified” refers to a construct described herein, or variant thereof that may be substantially or essentially free of components that normally accompany or interact with the protein as found in its naturally occurring environment, i.e. a native cell, or host cell in the case of recombinantly produced heteromultimer that in certain embodiments, is substantially free of cellular material includes preparations of protein having less than about 30%, less than about 25%, less than about 20%, less than about 15%, less than about 10%, less than about 5%, less than about 4%, less than about 3%, less than about 2%, or less than about 1% (by dry weight) of contaminating protein. When the heteromultimer or variant thereof is recombinantly produced by the host cells, the protein in certain embodiments is present at about 30%, about 25%, about 20%, about 15%, about 10%, about 5%, about 4%, about 3%, about 2%, or about 1% or less of the dry weight of the cells. When the heteromultimer or variant thereof is recombinantly produced by the host cells, the protein, in certain embodiments, is present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less of the dry weight of the cells. In certain embodiments, “substantially purified” heteromultimer produced by the methods described herein, has a purity level of at least about 30%, at least about 35%, at least about 40%, at least about 45%, at least about 50%, at least about 55%, at least about 60%, at least about 65%, at least about 70%, specifically, a purity level of at least about 75%, 80%, 85%, and more specifically, a purity level of at least about 90%, a purity level of at least about 95%, a purity level of at least about 99% or greater as determined by appropriate methods such as SDS/PAGE analysis, RP-HPLC, SEC, and capillary electrophoresis.

A “recombinant host cell” or “host cell” refers to a cell that includes an exogenous polynucleotide, regardless of the method used for insertion, for example, direct uptake, transduction, f-mating, or other methods known in the art to create recombinant host cells. The exogenous polynucleotide may be maintained as a nonintegrated vector, for example, a plasmid, or alternatively, may be integrated into the host genome.

As used herein, the term “medium” or “media” includes any culture medium, solution, solid, semi-solid, or rigid support that may support or contain any host cell, including bacterial host cells, yeast host cells, insect host cells, plant host cells, eukaryotic host cells, mammalian host cells, CHO cells, prokaryotic host cells, E. coli, or Pseudomonas host cells, and cell contents. Thus, the term may encompass medium in which the host cell has been grown, e.g., medium into which the protein has been secreted, including medium either before or after a proliferation step. The term also may encompass buffers or reagents that contain host cell lysates, such as in the case where a heteromultimer described herein is produced intracellularly and the host cells are lysed or disrupted to release the heteromultimer.

“Refolding,” as used herein describes any process, reaction or method which transforms disulfide bond containing polypeptides from an improperly folded or unfolded state to a native or properly folded conformation with respect to disulfide bonds.

“Cofolding,” as used herein, refers specifically to refolding processes, reactions, or methods which employ at least two monomeric polypeptides which interact with each other and result in the transformation of unfolded or improperly folded polypeptides to native, properly folded polypeptides.

As used herein, the term “modulated serum half-life” means the positive or negative change in circulating half-life of an antigen binding polypeptide that is comprised by an antibody construct described herein relative to its native form. Serum half-life is measured by taking blood samples at various time points after administration of the construct, and determining the concentration of that molecule in each sample. Correlation of the serum concentration with time allows calculation of the serum half-life. Increased serum half-life desirably has at least about two-fold, but a smaller increase may be useful, for example where it enables a satisfactory dosing regimen or avoids a toxic effect. In some embodiments, the increase is at least about three-fold, at least about five-fold, or at least about ten-fold.

The term “modulated therapeutic half-life” as used herein means the positive or negative change in the half-life of the therapeutically effective amount of an antigen binding polypeptide comprised by a monovalent antibody construct described herein, relative to its non-modified form. Therapeutic half-life is measured by measuring pharmacokinetic and/or pharmacodynamic properties of the molecule at various time points after administration. Increased therapeutic half-life desirably enables a particular beneficial dosing regimen, a particular beneficial total dose, or avoids an undesired effect. In some embodiments, the increased therapeutic half-life results from increased potency, increased or decreased binding of the modified molecule to its target, increased or decreased breakdown of the molecule by enzymes such as proteases, or an increase or decrease in another parameter or mechanism of action of the non-modified molecule or an increase or decrease in receptor-mediated clearance of the molecule.

The term “isolated,” when applied to a nucleic acid or protein, denotes that the nucleic acid or protein is free of at least some of the cellular components with which it is associated in the natural state, or that the nucleic acid or protein has been concentrated to a level greater than the concentration of its in vivo or in vitro production. It can be in a homogeneous state. Isolated substances can be in either a dry or semi-dry state, or in solution, including but not limited to, an aqueous solution. It can be a component of a pharmaceutical composition that comprises additional pharmaceutically acceptable carriers and/or excipients. Purity and homogeneity are typically determined using analytical chemistry techniques such as polyacrylamide gel electrophoresis or high performance liquid chromatography. A protein which is the predominant species present in a preparation is substantially purified. In particular, an isolated gene is separated from open reading frames which flank the gene and encode a protein other than the gene of interest. The term “purified” denotes that a nucleic acid or protein gives rise to substantially one band in an electrophoretic gel. Particularly, it may mean that the nucleic acid or protein is at least 85% pure, at least 90% pure, at least 95% pure, at least 99% or greater pure.

The term “nucleic acid” refers to deoxyribonucleotides, deoxyribonucleosides, ribonucleosides, or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless specifically limited otherwise, the term also refers to oligonucleotide analogs including PNA (peptidonucleic acid), analogs of DNA used in antisense technology (phosphorothioates, phosphoroamidates, and the like). Unless otherwise indicated, a particular nucleic acid sequence also implicitly encompasses conservatively modified variants thereof (including but not limited to, degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)).

The terms “polypeptide,” “peptide” and “protein” are used interchangeably herein to refer to a polymer of amino acid residues. That is, a description directed to a polypeptide applies equally to a description of a peptide and a description of a protein, and vice versa. The terms apply to naturally occurring amino acid polymers as well as amino acid polymers in which one or more amino acid residues is a non-naturally encoded amino acid. As used herein, the terms encompass amino acid chains of any length, including full length proteins, wherein the amino acid residues are linked by covalent peptide bonds.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, praline, serine, threonine, tryptophan, tyrosine, and valine) and pyrrolysine and selenocysteine. Amino acid analogs refers to compounds that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. Reference to an amino acid includes, for example, naturally occurring proteogenic L-amino acids; D-amino acids, chemically modified amino acids such as amino acid variants and derivatives; naturally occurring non-proteogenic amino acids such as β-alanine, ornithine, etc.; and chemically synthesized compounds having properties known in the art to be characteristic of amino acids. Examples of non-naturally occurring amino acids include, but are not limited to, α-methyl amino acids (e.g. α-methyl alanine), D-amino acids, histidine-like amino acids (e.g., 2-amino-histidine, β-hydroxy-histidine, homohistidine), amino acids having an extra methylene in the side chain (“homo” amino acids), and amino acids in which a carboxylic acid functional group in the side chain is replaced with a sulfonic acid group (e.g., cysteic acid). The incorporation of non-natural amino acids, including synthetic non-native amino acids, substituted amino acids, or one or more D-amino acids into the proteins of the present invention may be advantageous in a number of different ways. D-amino acid-containing peptides, etc., exhibit increased stability in vitro or in vivo compared to L-amino acid-containing counterparts. Thus, the construction of peptides, etc., incorporating D-amino acids can be particularly useful when greater intracellular stability is desired or required. More specifically, D-peptides, etc., are resistant to endogenous peptidases and proteases, thereby providing improved bioavailability of the molecule, and prolonged lifetimes in vivo when such properties are desirable. Additionally, D-peptides, etc., cannot be processed efficiently for major histocompatibility complex class II-restricted presentation to T helper cells, and are therefore, less likely to induce humoral immune responses in the whole organism.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of ordinary skill in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of ordinary skill in the art will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the deletion of an amino acid, addition of an amino acid, or substitution of an amino acid with a chemically similar amino acid. Conservative substitution tables providing functionally similar amino acids are known to those of ordinary skill in the art. Such conservatively modified variants are in addition to and do not exclude polymorphic variants, interspecies homologs, and alleles of the invention.

Conservative substitution tables providing functionally similar amino acids are known to those of ordinary skill in the art. The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and [0139] 8) Cysteine (C), Methionine (M) (see, e.g., Creighton, Proteins: Structures and Molecular Properties (W H Freeman & Co.; 2nd edition (December 1993)

The terms “identical” or percent “identity,” in the context of two or more nucleic acids or polypeptide sequences, refer to two or more sequences or subsequences that are the same. Sequences are “substantially identical” if they have a percentage of amino acid residues or nucleotides that are the same (i.e., about 60% identity, about 65%, about 70%, about 75%, about 80%, about 85%, about 90%, or about 95% identity over a specified region), when compared and aligned for maximum correspondence over a comparison window, or designated region as measured using one of the following sequence comparison algorithms (or other algorithms available to persons of ordinary skill in the art) or by manual alignment and visual inspection. This definition also refers to the complement of a test sequence. The identity can exist over a region that is at least about 50 amino acids or nucleotides in length, or over a region that is 75-100 amino acids or nucleotides in length, or, where not specified, across the entire sequence of a polynucleotide or polypeptide. A polynucleotide encoding a polypeptide of the present invention, including homologs from species other than human, may be obtained by a process comprising the steps of screening a library under stringent hybridization conditions with a labeled probe having a polynucleotide sequence of the invention or a fragment thereof, and isolating full-length cDNA and genomic clones containing said polynucleotide sequence. Such hybridization techniques are well known to the skilled artisan.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are known to those of ordinary skill in the art. Optimal alignment of sequences for comparison can be conducted, including but not limited to, by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

One example of an algorithm that is suitable for determining percent sequence identity and sequence similarity are the BLAST and BLAST 2.0 algorithms, which are described in Altschul et al. (1997) Nuc. Acids Res. 25:3389-3402, and Altschul et al. (1990) J. Mol. Biol. 215:403-410, respectively. Software for performing BLAST analyses is publicly available through the National Center for Biotechnology Information available at the World Wide Web at ncbi.nlm.nih.gov. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1992) Proc. Natl. Acad. Sci. USA 89:10915) alignments (B) of 50, expectation (E) of 10, M=5, N=−4, and a comparison of both strands. The BLAST algorithm is typically performed with the “low complexity” filter turned off.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, or less than about 0.01, or less than about 0.001.

The phrase “selectively (or specifically) hybridizes to” refers to the binding, duplexing, or hybridizing of a molecule only to a particular nucleotide sequence under stringent hybridization conditions when that sequence is present in a complex mixture (including but not limited to, total cellular or library DNA or RNA).

The phrase “stringent hybridization conditions” refers to hybridization of sequences of DNA, RNA, or other nucleic acids, or combinations thereof under conditions of low ionic strength and high temperature as is known in the art. Typically, under stringent conditions a probe will hybridize to its target subsequence in a complex mixture of nucleic acid (including but not limited to, total cellular or library DNA or RNA) but does not hybridize to other sequences in the complex mixture. Stringent conditions are sequence-dependent and will be different in different circumstances. Longer sequences hybridize specifically at higher temperatures. An extensive guide to the hybridization of nucleic acids is found in Tijssen, Laboratory Techniques in Biochemistry and Molecular Biology—Hybridization with Nucleic Probes, “Overview of principles of hybridization and the strategy of nucleic acid assays” (1993).

As used herein, the term “eukaryote” refers to organisms belonging to the phylogenetic domain Eucarya such as animals (including but not limited to, mammals, insects, reptiles, birds, etc.), ciliates, plants (including but not limited to, monocots, dicots, algae, etc.), fungi, yeasts, flagellates, microsporidia, protists, etc.

As used herein, the term “prokaryote” refers to prokaryotic organisms. For example, a non-eukaryotic organism can belong to the Eubacteria (including but not limited to, Escherichia coli, Thermus thermophilus, Bacillus stearothermophilus, Pseudomonas fluorescens, Pseudomonas aeruginosa, Pseudomonas putida, etc.) phylogenetic domain, or the Archaea (including but not limited to, Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, etc.) phylogenetic domain.

The term “subject” as used herein, refers to an animal, in some embodiments a mammal, and in other embodiments a human, who is the object of treatment, observation or experiment. An animal may be a companion animal (e.g., dogs, cats, and the like), farm animal (e.g., cows, sheep, pigs, horses, and the like) or a laboratory animal (e.g., rats, mice, guinea pigs, and the like).

The term “effective amount” as used herein refers to that amount of monovalent antibody construct being administered, which will relieve to some extent one or more of the symptoms of the disease, condition or disorder being treated. Compositions containing the construct described herein can be administered for prophylactic, enhancing, and/or therapeutic treatments.

The terms “enhance” or “enhancing” means to increase or prolong either in potency or duration a desired effect. Thus, in regard to enhancing the effect of drug molecule or therapeutic agents, the term “enhancing” refers to the ability to increase or prolong, either in potency or duration, the effect of therapeutic agents on a system. An “enhancing-effective amount,” as used herein, refers to an amount adequate to enhance the effect of another therapeutic agent or drug in a desired system. When used in a patient, amounts effective for this use will depend on the severity and course of the disease, disorder or condition, previous therapy, the patient's health status and response to the drugs, and the judgment of the treating physician.

The term “modified,” as used herein refers to any changes made to a given polypeptide, such as changes to the length of the polypeptide, the amino acid sequence, chemical structure, co-translational modification, or post-translational modification of a polypeptide. The form “(modified)” term means that the polypeptides being discussed are optionally modified, that is, the polypeptides under discussion can be modified or unmodified.

The term “post-translationally modified” refers to any modification of a natural or non-natural amino acid that occurs to such an amino acid after it has been incorporated into a polypeptide chain. The term encompasses, by way of example only, co-translational in vivo modifications, co-translational in vitro modifications (such as in a cell-free translation system), post-translational in vivo modifications, and post-translational in vitro modifications.

The term “monospecific bivalent antibody construct” as used herein refers to an antibody construct which has two antigen binding domains (bivalent), both of which bind to the same epitope/antigen (monospecific). The antigen binding domains could be, but are not limited to, protein constructs such as Fab (fragment antigen binding), scFv (single chain Fv) and sdab (single domain antibody). The monospecific bivalent antibody construct is also referred to herein as a “full-size antibody” or “FSA.” The monospecific bivalent antibody construct is a reference against which the properties of the monovalent antibody constructs are measured.

The term “avidity” is used here to refer to the combined synergistic strength of binding affinities and a key structure and biological attribute of therapeutic monospecific bivalent antibodies. Lack of avidity and loss of synergistic strength of binding can result in reduced apparent target binding affinity. On the other hand, on a target cell with fixed number of antigens, avidity resulting from the multivalent (or bivalent) binding causes increased occupancy of the target antigen at a lower number of antibody molecules relative to antibody which displays monovalent binding. With a lower number of antibody molecules bound to the target cell, in the application of bivalent lytic antibodies, antibody dependent cytotoxic killing mechanisms may not occur efficiently resulting in reduced efficacy. Not enough antibodies are bound to mediate ADCC as ADCC, CDC, ADCP are generally considered to be Fc concentration threshold dependent. In the case of agonistic antibodies, reduced avidity reduces their efficiency to crosslink and dimerize antigens and activate the pathway.

“Single domain antibodies” or “Sdab”—Single domain antibodies such as the Camelid VhH domain are individual immunoglobulin domains. Sdabs are fairly stable and easy to express as fusion partner with the Fc chain of an antibody (Harmsen M M, De Haard H J (2007). “Properties, production, and applications of camelid single-domain antibody fragments”. Appl. Microbiol Biotechnol. 77(1): 13-22).

A “HER receptor” is a receptor protein tyrosine kinase which belongs to the human epidermal growth factor receptor (HER) family and includes EGFR, HER2, HER3 and HER4 receptors. The HER receptor will generally comprise an extracellular domain, which may bind an HER ligand; a lipophilic transmembrane domain; a conserved intracellular tyrosine kinase domain; and a carboxyl-terminal signaling domain harboring several tyrosine residues which can be phosphorylated.

The extracellular (ecto) domain of HER2 comprises four domains. Domain I (ECD1, amino acid residues from about 1-195), Domain II (ECD2, amino acid residues from about 196-319). Domain III (ECD3, amino acid residues from about 320-488), and Domain IV (ECD4, amino acid residues from about 489-630) (residue numbering without signal peptide). See Garrett et al. Mol. Cell. 11: 495-505 (2003). Cho et al. Nature 421: 756-760 (2003), Franklin et al. Cancer Cell 5:317-328 (2004). Tse et al. Cancer Treat Rev. 2012 April; 38(2):133-42 (2012), or Plowman et al. Proc. Natl. Acad. Sci. 90:1746-1750 (1993).

The expressions “ErbB2” and “HER2” are used interchangeably herein and refer to human HER2 protein described, for example, in Semba et al., PNAS (USA) 82:6497-6501 (1985) and Yamamoto et al. Nature 319:230-234 (1986) (Genebank accession number X03363). The term “erbB2” and “neu” refers to the gene encoding human ErbB2 protein. p185 or p185neu refers to the protein product of the neu gene. Preferred HER2 is native sequence human HER2.

By “HER ligand” is meant a polypeptide which binds to and/or activates an HER receptor. The HER ligand of particular interest herein is a native sequence human HER ligand such as epidermal growth factor (EGF) (Savage et al., J. Biol. Chem. 247:7612-7621 (1972)); transforming growth factor alpha (TGF-α) (Marquardt et al., Science 223:1079-1082 (1984)); amphiregulin also known as schwanonma or keratinocyte autocrine growth factor (Shoyab et al. Science 243:1074-1076 (1989); Kimura et al. Nature 348:257-260 (1990); and Cook et al. Mol. Cell. Biol. 11:2547-2557 (1991)); betacellulin (Shing et al., Science 259:1604-1607 (1993); and Sasada et al. Biochem. Biophys. Res. Conunun. 190:1173 (1993)); heparin-binding epidermal growth factor (HB-EGF) (Higashiyama et al., Science 251:936-939 (1991)); epiregulin (Toyoda et al., J. Biol. Chem. 270:7495-7500 (1995); and Komurasaki et al. Oncogene 15:2841-2848 (1997)); a heregulin (see below); neuregulin-2 (NRG-2) (Carraway et al., Nature 387:512-516 (1997)); neuregulin-3 (NRG-3) (Zhang et al., Proc. Natl. Acad. Sci. 94:9562-9567 (1997)); neuregulin-4 (NRG-4) (Harari et al. Oncogene 18:2681-89 (1999)) or cripto (CR-1) (Kannan et al. J. Biol. Chem. 272(6):3330-3335 (1997)). HER ligands which bind EGFR include EGF, TGF-α, amphiregulin, betacellulin, HB-EGF and epiregulin. HER ligands which bind HER3 include heregulins. HER ligands capable of binding HER4 include betacellulin, epiregulin, HB-EGF, NRG-2, NRG-3, NRG-4 and heregulins.

“Heregulin” (HRG) when used herein refers to a polypeptide encoded by the heregulin gene product as disclosed in U.S. Pat. No. 5,641,869 or Marchionni et al., Nature, 362:312-318 (1993). Examples of heregulins include heregulin-α, heregulin-β1, heregulin-β2 and heregulin-β3 (Holmes et al., Science, 256:1205-1210 (1992); and U.S. Pat. No. 5,641,869); neu differentiation factor (NDF) (Peles et al. Cell 69: 205-216 (1992)); acetylcholine receptor-inducing activity (ARIA) (Falls et al. Cell 72:801-815 (1993)); glial growth factors (GGFs) (Marchionni et al., Nature, 362:312-318 (1993)); sensory and motor neuron derived factor (SMDF) (Ho et al. J. Biol. Chem. 270:14523-14532 (1995)); γ-heregulin (Schaefer et al. Oncogene 15:1385-1394 (1997)). The term includes biologically active fragments and/or amino acid sequence variants of a native sequence HRG polypeptide, such as an EGF-like domain fragment thereof (e.g. HRGβ1177-244).

“HER activation” or “HER2 activation” refers to activation, or phosphorylation, of any one or more HER receptors, or HER2 receptors. Generally, HER activation results in signal transduction (e.g. that caused by an intracellular kinase domain of a HER receptor phosphorylating tyrosine residues in the HER receptor or a substrate polypeptide). HER activation may be mediated by HER ligand binding to a HER dimer comprising the HER receptor of interest. HER ligand binding to a HER dimer may activate a kinase domain of one or more of the HER receptors in the dimer and thereby results in phosphorylation of tyrosine residues in one or more of the HER receptors and/or phosphorylation of tyrosine residues in additional substrate polypeptides(s), such as Akt or MAPK intracellular kinases.

Antibody “effector functions” refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g. B cell receptor: BCR), etc.

The “Fab fragment” of an antibody (also referred to as fragment antigen binding) contains the constant domain (CL) of the light chain and the first constant domain (CH1) of the heavy chain along with the variable domains VL and VH on the light and heavy chains respectively. The variable domains comprise the complementarity determining loops (CDR, also referred to as hypervariable region) that are involved in antigen binding. Fab′ fragments differ from Fab fragments by the addition of a few residues at the carboxy terminus of the heavy chain CH1 domain including one or more cysteines from the antibody hinge region.

“Single-chain Fv” or “scFv” antibody fragments comprise the VH and VL domains of an antibody, wherein these domains are present in a single polypeptide chain. In one embodiment, the Fv polypeptide further comprises a polypeptide linker between the VH and VL domains which enables the scFv to form the desired structure for antigen binding. For a review of scFv see Pluckthun in The Pharmacology of Monoclonal Antibodies, vol. 113, Rosenburg and Moore eds., Springer-Verlag, New York, pp. 269-315 (1994). HER2 antibody scFv fragments are described in WO93/16185; U.S. Pat. No. 5,571,894; and U.S. Pat. No. 5,587,458.

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986): Riechmann et al., Nature 332:323-329 (1988); and Presta, Curr. Op. Struct. Biol. 2:593-596 (1992).

Humanized HER2 antibodies include huMAb4D5-1, huMAb4D5-2, huMAb4D5-3, huMAb4D5-4, huMAb4D5-5, huMAb4D5-6, huMAb4D5-7 and huMAb4D5-8 or Trastuzumab (HERCEPTIN®) as described in Table 3 of U.S. Pat. No. 5,821,337 expressly incorporated herein by reference; humanized 520C9 (WO93/21319) and 20′ humanized 2C4 antibodies as described in US Patent Publication No. 2006/0018899.

The “epitope 2C4” is the region in the extracellular domain of HER2 to which the antibody 2C4 binds. In order to screen for antibodies which bind to the 2C4 epitope, a routine cross-blocking assay such as that described in Antibodies, A Laboratory Manual. Cold Spring Harbor Laboratory, Ed Harlow and David Lane (1988), can be performed. Alternatively, epitope mapping can be performed to assess whether the antibody binds to the 2C4 epitope of HER2 using methods known in the art and/or one can study the antibody-HER2 structure (Franklin et al. Cancer Cell 5:317-328 (2004)) to see what domain(s) of HER2 is/are bound by the antibody. Epitope 2C4 comprises residues from domain II in the extracellular domain of HER2. 2C4 and Pertuzumab bind to the extracellular domain of HER2 at the junction of domains I, II and III. Franklin et al. Cancer Cell 5:317-328 (2004).

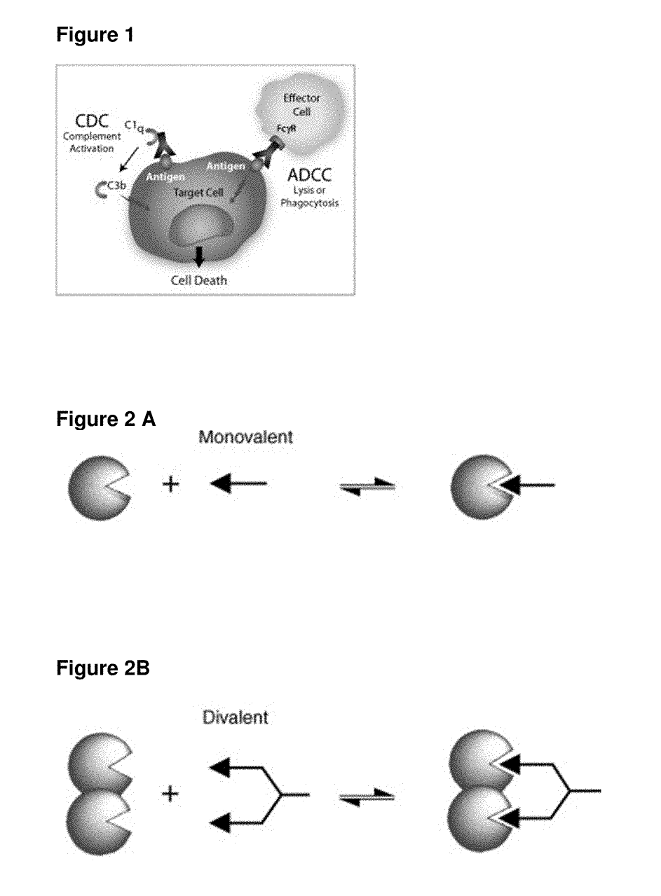

The “epitope 4D5” is the region in the extracellular domain of HER2 to which the antibody 4D5 (ATCC CRL 10463) and Trastuzumab bind. This epitope is close to the transmembrane domain of HER2, and within Domain IV of HER2. To screen for antibodies which bind to the 4D5 epitope, a routine cross-blocking assay such as that described in Antibodies, A Laboratory Manual, Cold Spring Harbor Laboratory. Ed Harlow and David Lane (1988), can be performed. Alternatively, epitope mapping can be performed to assess whether the antibody binds to the 4D5 epitope of HER2 (e.g. any one or more residues in the region from about residue 529 to about residue 625, inclusive, see FIG. 1 of US Patent Publication No. 2006/0018899).

The “epitope 7C2/F3” is the region at the N terminus, within Domain I, of the extracellular domain of HER2 to which the 7C2 and/or 7F3 antibodies (each deposited with the ATCC, see below) bind. To screen for antibodies which bind to the 7C2/7F3 epitope, a routine cross-blocking assay such as that described in Antibodies, A Laboratory Manual, Cold Spring Harbor Laboratory. Ed Harlow and David Lane (1988), can be performed. Alternatively, epitope mapping can be performed to establish whether the antibody binds to the 7C2/7F3 epitope on HER2 (e.g. any one or more of residues in the region from about residue 22 to about residue 53 of HER2, see FIG. 1 of US Patent Publication No. 2006/0018899).

The term “antigen modulation” as used herein refers to a change or loss of surface receptor density via internalization or down regulation) such as in the ADC.

Antigen-Binding Polypeptide Construct

The antigen-binding polypeptide construct which monovalently binds an antigen can be derived from known antibodies or antigen-binding domains, or can be derived from novel antibodies or antigen-binding domains. The identification of an antigen-binding polypeptide construct for the monovalent antibody construct is based on the selection of the target cell and on the selection of an antigen expressed on the surface of the target cell. For example, once the target cell has been selected, an antigen is then selected that is a) expressed on the cell surface of the target cell, but not expressed on the surface of other cells, or b) expressed at higher levels on the cell surface of the target cell, but expressed at lower levels on the surface of other cells. This allows for selective targeting of the target cell.

Selection of Target Cells

The target cell is selected based on the intended use of the monovalent antibody construct. In one embodiment, the target cell is a cell which is activated or amplified in a cancer, an infectious disease, an autoimmune disease, or in an inflammatory disease.

In one embodiment, where the monovalent antibody construct is intended for use in the treatment of cancer, the target cell is derived from a tumor that exhibits HER2 3+ overexpression. In one embodiment, the target cell is derived from a tumor that exhibits HER2 low expression. In one embodiment, the target cell is derived from a tumor that exhibits HER2 resistance. In one embodiment, the target cell is derived from a tumor that is a triple negative (ER/PR/HER2) tumor.

In embodiments where the monovalent antibody construct is intended for use in the treatment of cancer, the target cell is a cancer cell line that is representative of HER2 3+ overexpression eg. SKBR3, BT474. In one embodiment, the target cell is a cancer cell line that is representative of HER2 low expression eg. MCF7. In one embodiment, the target cell is a cancer cell line that is representative of HER2 resistance eg. JIMT1. In one embodiment, the target cell is a cancer cell line that is representative of breast cancer triple negative eg. MDA-MD-231 cells.

In one embodiment, the monovalent antibody construct according to the invention is designed to target a breast cancer cell. Exemplary classes of breast cancer cells include but are not limited to the following: progesterone receptor (PR) negative and estrogen receptor (ER) negative cells, low HER 2-expressing cells, medium HER2-expressing cells, high HER2-expressing cells, or anti-HER2 antibody resistant cells.

In one embodiment, the monovalent antibody construct described herein is designed to target Gastric and Esophageal Adenocarcinomas. Exemplary histologic types include: HER2 positive proximal gastric carcinomas with intestinal phenotype and HER2 positive distal diffuse gastric carcinomas. Exemplary classes of gastric cancer cells include but are not limited to (N-87, OE-19, SNU-216 and MKN-7).

In another embodiment, a monovalent antibody construct described herein is designed to target Metastatic HER2+ Breast Cancer Tumors in the Brain. Exemplary classes of gastric cancer cells include but are not limited to BT474 (as above for breast cancer).

Selection of Antigen

As indicated above, the antigen to which the antigen-binding polypeptide construct binds is selected depending on the target cell the monovalent antibody construct is intended to bind to. In one embodiment, the antigen to which the antigen-binding polypeptide construct binds is selected based on 1) increased expression on the surface of the target cell or b) selective expression on the surface of the target cell compared to the surface of other cells. Accordingly, in one embodiment, the monovalent antibody construct is designed to target one of the target cell types listed in Table A1.

TABLE A1
List of antibodies and respective target cells

| Antibody/target | Target cell |
|---|---|
| αCD16a | NK cells, Macrophages |
| αCD30 | Activated T-cells |
| αCD137/4-1BB | T-cells |
| αCD22 | B-cells |
| αCD52 | B-cells |
| αCD80 | B-cells |
| αCD23 | B-cell antigen |
| αCD2 | T-cells |
| CD4 | T-cell marker. Binds MHC II |
| CD40 | B-cell co-stimulatory receptor |
| αKIR | NK cells |
| αCD32b | B-cells, monocytes, macrophages |
| αEpCam | TAA |
| αEGFR | TAA |
| αCD25/IL2R | activated T-cells |
| αCEA | TAA |
| αGP100 | TAA |
| αLAG3 | activated T-cells |
| αB7-H3/CD276 | T-cells |
| αCTLA4 | T-cell |
| αVEGFR | VEGFR-1 is required for the recruitment of |
| | haematopoietic stem cells and the migration of |
| | monocytes and macrophages |
| | VEGFR-2 regulates vascular endothelial |
| | function |
| | VEGFR-3 regulates lymphatic endothelial cell |
| | function |

Table A1 additionally identifies known antibodies that can be used to target the cell types listed, and by extension also identifies the antigen expressed on the desired target cell. For example, “αCD16a” in Table A1 indicates that an antibody to CD16a can be used to target NK cells and macrophages. In certain embodiments, the monovalent antibody construct described herein comprises an antigen-binding polypeptide construct that is derived from the antigen-binding domain of one of the antibodies listed in Table A1.

In embodiments where the monovalent antibody construct according to the invention is designed to target a breast cancer cell, the antigen-binding polypeptide construct monovalently binds an antigen that is expressed on the surface of the breast cancer cell. Suitable antigens include, but are not limited to HER2. In one embodiment, the epitope that the antigen-binding polypeptide construct binds to an extracellular domain of the target antigen on the target cell.

In embodiments where the monovalent antibody construct comprises an antigen-binding polypeptide construct that binds to HER2, the antigen-binding polypeptide construct binds to HER2 or to a particular domain or epitope of HER2. In one embodiment, the antigen-binding polypeptide construct binds to an extracellular domain of HER2. As is known in the art, the HER2 antigen comprises multiple extracellular domains (ECDs).

In one embodiment is a monovalent antibody construct described herein which comprises an antigen-binding polypeptide construct that binds to an ECD of HER2 selected from ECD1, ECD2, ECD3, and ECD4. In another embodiment, the monovalent antibody construct comprises an antigen-binding polypeptide construct that binds to an ECD of HER2 selected from ECD1, ECD2, and ECD4. In one embodiment, the monovalent antibody construct comprises an antigen-binding polypeptide construct that binds to ECD1. In one embodiment, the monovalent antibody construct comprises an antigen-binding polypeptide construct that binds to ECD2. In one embodiment, the monovalent antibody construct comprises an antigen-binding polypeptide construct that binds to ECD4. In another embodiment, the monovalent antibody construct comprises an antigen-binding polypeptide construct that binds to an epitope of HER2 selected from 2C4 (eg. OA1-Fab-Her2), 4D5 (OA3-scFv-Her2) and C6.5 (OA4-scFv-BID2).

Selection of Antibodies

In embodiments where the monovalent antibody construct comprises an antigen-binding polypeptide construct that binds to HER2, the antigen-binding polypeptide construct can be derived from known anti-HER2 antibodies or anti-HER2 binding domains in various formats including Fab fragments, scFvs, and sdab. In certain embodiments the antigen-binding polypeptide construct can be derived from humanized, or chimeric versions of these antibodies. In one embodiment, the antigen-binding polypeptide construct is derived from a Fab fragment of trastuzumab, pertuzumab, or humanized versions thereof. In one embodiment, the antigen-binding polypeptide construct is derived from an scFv. Non-limiting examples of such antigen-binding polypeptide constructs include those found in the monovalent antibody constructs OA3-scFv-Her2 and OA4-scFv-BID2. In one embodiment, the antigen-binding polypeptide construct is derived from an sdab.

Dimeric/Heterodimeric Fc Construct

The monovalent antibody constructs according to the invention comprise a dimeric Fc polypeptide construct comprising two monomeric Fc polypeptides, each comprising a CH3 domain. In one embodiment of the invention, the dimeric Fc polypeptide construct is heterodimeric and comprises monomeric Fc polypeptides that have been modified promote the formation of a heterodimeric Fc. In one embodiment, the monomeric Fc polypeptides comprise variant CH3 domains having amino acid modifications that promote the formation of heterodimeric Fc domains. Suitable variant CH3 domains are known in the art and include, for example, those described in International Patent Publication No. WO 2012/058768, U.S. Pat. Nos. 5,821,333, 7,695,936 [KiH]. In one embodiment, the heteromultimer according to the invention comprises an IgG FcD construct wherein one of said first and second Fc polypeptides comprises the CH3 amino acid modifications T366L/N390R/K392R/T394W and the other Fc polypeptide comprises the CH3 amino acid modifications L351Y/S400E/F405A/Y407V.

Although monovalent constructs such as scFv, Fab, domain antibody have been known in the art, these monovalent constructs lack an Fc domain that is active for effector activity. Monovalent antigen binding constructs that comprise of a single chain of Fc which does not dimerize (homodimerize nor heterodimerize) are also known in literature [Engineering a Monomeric Fc Modality by N-Glycosylation for the Half-Life Extension of Biotherapeutics. Ishino T, Wang M, Mosyak L, Tam A, Duan W, Svenson K, Joyce A, O'Hara D M, Lin L, Somers W S, Kriz R. J Biol Chem. 2013 Apr. 24. PMID: 23615911] but unlike constructs according to this invention, these constructs also lack immune effector functionality that is dependent on the dimeric Fc domain.

Additional methods for modifying monomeric Fc polypeptides to promote heterodimeric Fc formation are described in International Patent Publication No. WO 96/027011 (knobs into holes), in Gunasekaran et al. (Gunasekaran K. et al. (2010) J Biol Chem. 285, 19637-46, electrostatic design to achieve selective heterodimerization), in Davis et al. (Davis, J H. et al. (2010) Prot Eng Des Sel; 23(4): 195-202, strand exchange engineered domain (SEED) technology), and in Labrijn et al [Efficient generation of stable bispecific IgG1 by controlled Fab-arm exchange. Labrijn A F, Meesters J I, de Goeij B E, van den Bremer E T, Neijssen J, van Kampen M D, Strumane K, Verploegen S, Kundu A, Gramer M J, van Berkel P H, van de Winkel J G, Schuurman J, Parren P W. Proc Natl Acad Sci USA. 2013 Mar. 26; 110(13):5145-50.

In some embodiments, the modified monomeric Fc polypeptides further comprise amino acid modifications that increase the stability of the heterodimeric Fc polypeptide construct, as determined by its melting temperature. Suitable amino acid modifications are known in the art and include, for example, those described in International Patent Application No. PCT/CA2012/050780. Specifically, in one embodiment, the heterodimeric Fc polypeptide construct comprises modified monomeric Fc polypeptides with the amino acid modification T350V in both peptides.

In some embodiments is an isolated monovalent antibody construct described herein comprising an antigen-binding polypeptide construct which monovalently binds an antigen; and a dimeric Fc polypeptide construct comprising a variant CH3 domain. In some embodiments, the variant CH3 domain comprises amino acid mutations that promote the formation of said heterodimer with stability comparable to a native homodimeric Fc region. In some embodiments, the variant CH3 domain has a melting temperature (Tₘ) of about 70° C. or higher. In some embodiments the variant CH3 domain has a melting temperature (Tₘ) of about 75° C. or higher. In select embodiments, the variant CH3 domain has a melting temperature (Tₘ) of about 80° C. or higher.

In some embodiments is an isolated monovalent antibody construct described herein comprising an antigen-binding polypeptide construct which monovalently binds an antigen; and a dimeric Fc polypeptide construct comprising a CH3 domain wherein the Fc construct does not comprise an additional disulfide bond in the CH3 domain relative to a wild type Fc region. In certain embodiments the Fc construct comprises an additional disulfide bond in the variant CH3 domain relative to a wild type Fc region, and wherein the variant CH3 domain has a melting temperature (Tₘ) of at least about 77.5° C. In specific embodiments, the dimeric Fc construct is a heterodimeric Fc construct formed with a purity greater than about 75%. In some embodiments, the dimeric Fc construct is a heterodimeric Fc construct formed with a purity greater than about 80%. In certain embodiments, the dimeric Fc construct is a heterodimeric Fc construct formed with a purity greater than about 90%. In some other embodiments the dimeric Fc construct is a heterodimeric Fc construct formed with a purity greater than about 95%.

In some embodiments is an isolated monovalent antibody construct described herein comprising an antigen-binding polypeptide construct which monovalently binds an antigen; and a dimeric Fc polypeptide construct that has superior biophysical properties like stability and easy to manufacture relative to a monovalent antigen binding polypeptide which is not fused to the Fc polypeptide.

FcRn Binding and PK Parameters

As is known in the art, binding to FcRn recycles endocytosed antibody from the endosome back to the bloodstream (Raghavan et al., 1996, Annu Rev Cell Dev Biol 12:181-220; Ghetie et al., 2000, Annu Rev Immunol 18:739-766). This process, coupled with preclusion of kidney filtration due to the large size of the full-length molecule, results in favorable antibody serum half-lives ranging from one to three weeks. Binding of Fc to FcRn also plays a key role in antibody transport. Thus, in one embodiment, the monovalent antibody constructs of the invention are able to bind FcRn.

Additional Modifications to Improve Effector Function.

In some embodiments is an isolated monovalent antibody construct described herein comprising an antigen-binding polypeptide construct which monovalently binds an antigen; and a dimeric Fc polypeptide construct comprising a CH3 domain and further comprising a variant CH2 domain. In some embodiments the variant CH2 domain is comprising asymmetric amino acid modifications to promote selective binding of a FcγR. In some embodiment the variant CH2 domain allows for separation and purification of the isolated monovalent antibody described herein.

In some embodiment is an isolated monovalent antibody construct described herein comprising an antigen binding polypeptide that monovalently binds an antigen; and wherein the antigen binding polypeptide is fused via a polypeptide to a monomeric Fc polypeptide comprising CH2 and CH3 domains.

In some embodiment is an isolated monovalent antibody construct described herein comprising an antigen binding polypeptide that monovalently binds an antigen; and wherein the antigen binding polypeptide is a Fab, wherein the heavy chain of the Fab is fused via a polypeptide to a monomeric Fc polypeptide comprising CH2 and CH3 domains and the light chain of the Fab is fused via a polypeptide to a second monomeric Fc polypeptide comprising CH2 and CH3 domains.

In some embodiment is an isolated monovalent antibody construct described herein comprising an antigen binding polypeptide that monovalently binds an antigen; and where in the antigen binding polypeptide is fused to a monomeric Fc polypeptide comprising CH2 and CH3 domains and a second polypeptide incapable of binding to any antigen; wherein the second polypeptide is fused to the second monomeric Fc polypeptide comprising the CH2 and CH3 domains; wherein the two monomeric Fc polypeptides pair to form a dimer.

In some embodiments the monovalent antibody constructs according to the invention may be modified to improve their effector function. Such modifications are known in the art and include afucosylation, or engineering of the affinity of the Fc portion of antibodies towards the activating receptors, mainly FCGR3a for ADCC, and towards C1q, for CDC. The following table summarizes the different designs reported in the literature for effector function engineering.

| Reference | Mutations | Effect |
|---|---|---|
| Lu, 2011, Ferrara | Afucosylated | Increased |
| 2011, Mizushima | | ADCC |
| 2011 | | |
| Lu, 2011 | S298A/E333A/K334A | Increased |
| | | ADCC |
| Lu, 2011 | S298A/E333A/K334A/K326A | Increased |
| | | ADCC |
| Stavenhagen, 2007 | F243L/R292P/Y300L/V305I/P396L | Increased |
| | | ADCC |
| Nordstrom, 2011 | F243L/R292P/Y300L/L235V/P396L | Increased |
| | | ADCC |
| Stewart, 2011 | F243L | Increased |
| | | ADCC |
| Shields, 2001 | S298A/E333A/K334A | Increased |
| | | ADCC |
| Lazar, 2006 | S239D/I332E/A330L | Increased |
| | | ADCC |
| Lazar, 2006 | S239D/I332E | Increased |
| | | ADCC |
| Bowles, 2006 | AME-D, not specified mutations | Increased |
| | | ADCC |
| Heider, 2011 | 37.1, mutations not disclosed | Increased |
| | | ADCC |
| Moore, 2010 | S267E/H268F/S324T | Increased |
| | | CDC |

Thus, in one embodiment, the monovalent antibody constructs can include a dimeric Fc polypeptide construct that comprises one or more amino acid modifications as noted in the above table that confer improved effector function. In another embodiment, the monovalent antibody construct are afucosylated to improve effector function.

In instances where it is desirable to increase the affinity of the antigen-binding polypeptide construct for its cognate antigen, methods known in the art can be used to increase the affinity of the antigen-binding polypeptide construct for its antigen. Examples of such methods are described in the following references, Birtalan et al. (2008) JMB 377, 1518-1528: Gerstner et al. (2002) JMB 321, 851-862; Kelley et al. (1993) Biochem 32(27), 6828-6835: Li et al. (2010) JBC 285(6). 3865-3871, and Vajdos et al. (2002) JMB 320, 415-428.

One example, of such a method is affinity maturation. One exemplary method for affinity maturation of HER2 antigen-binding domains is described as follows. Structures of the trastuzumab/HER2 (PDB code 1N8Z) complex and pertuzumab/HER2 complex (PDB code 1578) are used for modeling. Molecular dynamics (MD) can be employed to evaluate the intrinsic dynamic nature of the WT complex in an aqueous environment. Mean field and dead-end elimination methods along with flexible backbones can be used to optimize and prepare model structures for the mutants to be screened. Following packing a number of features will be scored including contact density, clash score, hydrophobicity and electrostatics. Generalized Born method will allow accurate modeling of the effect of solvent environment and compute the free energy differences following mutation of specific positions in the protein to alternate residue types. Contact density and clash score will provide a measure of complementarity, a critical aspect of effective protein packing. The screening procedure employs knowledge-based potentials as well as coupling analysis schemes relying on pair-wise residue interaction energy and entropy computations. Literature mutations known to enhance HER2 binding, and combinations of thereof are summarized in the following tables:

TABLE 1B
Trastuzumab mutations known to increase binding to HER2
for the Trastuzumab-HER2 system.

| Mutation | Reported Improvement |
|---|---|
| H_D102W (H_D98W) | 3.2X |
| H_D102Y | 3.1X |
| H_D102K | 2.3X |
| H_D102T | 2.2X |
| H_N55K | 2.0X |
| H_N55T | 1.9X |
| L_H91F | 2.1X |
| L_D28R | 1.9X |

TABLE 1C
Pertuzumab mutations known to increase binding to HER2
for the Pertuzumab-HER2 system.

| Mutation | Reported Improvement |
|---|---|
| L_I31A | 1.9X |
| L_Y96A | 2.1X |
| L_Y96F | 2.5X |
| H_T30A | 2.1X |
| H_G56A | 8.3X |
| H_F63V | 1.9X |

The monovalent antibody constructs described herein are internalized once they bind to the target cell. In one embodiment, the monovalent antibody constructs are internalized to a similar degree compared to the corresponding monospecific bivalent antibody constructs. In some embodiments, the monovalent antibody constructs are internalized more efficiently compared to the corresponding monospecific bivalent antibody constructs.

Increased Bmax and KD/on-Off Rate

Bmax is achieved at saturating antibody concentrations and Kd (on and off rate of an antibody) contributes to Bmax. An antibody with a slow on and fast off would have lower apparent Bmax compared to an antibody with a fast on and slow off rate of binding. For the monovalent antibody constructs according to the invention, the clearest separation in Bmax versus FSA occurs at saturating concentrations and where Bmax can no longer be increased with a FSA. The significance is less at non-saturating concentrations. In one embodiment the increase in Bmax and KD/on-off rate of the monovalent antibody construct compared to the monospecific bivalent antibody construct is independent of the level of target antigen expression on the target cell. In one embodiment, where the monovalent antibody construct comprises an antigen-binding polypeptide construct that binds to HER2, the increase in Bmax and KD/on-off rate of the monovalent antibody construct compared to the monospecific bivalent antibody construct is independent of the level of HER2 expression on the target cell.

In some embodiments is an isolated monovalent antibody construct described herein, wherein said monovalent antibody construct displays an increase in binding density and Bmax (maximum binding) to a target cell displaying said antigen as compared to a corresponding monospecific bivalent antibody construct with two antigen binding regions. In some embodiments said increase in binding density and Bmax is at least about 125% of the binding density and Bmax of the corresponding bivalent antibody construct. In certain embodiments, the increase in binding density and Bmax is at least about 150% of the binding density and Bmax of the corresponding bivalent antibody construct. In some embodiments, the increase in binding density and Bmax is at least about 200% of the binding density and Bmax of the corresponding bivalent antibody construct. In some embodiments, the increase in binding density and Bmax is greater than about 110% of the binding density and Bmax of the corresponding bivalent antibody construct.

Simply, agonism is the result of binding of an agent with intrinsic activity to some receptor on a cell which triggers an biochemical/biological effect. Agonists have been identified for many cell surface protein families including TRKs (tyrosine receptor kinases). For TRKs, agonist binding promotes receptor heterodimerization which triggers downstream signaling events. The extent of the biological effect is termed efficacy. Agonism can be assessed by both proximal biochemical markers such as receptor phosphorylation or distal biomarkers such as cell proliferation. In the context of a MV-L or MV-Int, some degree of agonism may be acceptable if this is overcomed by the antibody mediated cytotoxicity killing MOAs. In the case of MV-Int, some degree of agonism may increase the internalization rate and extent thereby increasing MV-Int intracellular levels and delivery of toxic payload to kill the cell.

Cross-linking and dimerization of receptors by a bivalent antibody mimics a cognate agonists actions on the target receptor. The efficiency of crosslinking is typically associated with efficacy. In the case of MV-L and MV-Int, the monovalent binding could not crosslink receptors as a FSA. However, data shows that monovalent antibodies can induce some agonist effects such as an impact on receptor phosphorylation or cell proliferation.

In certain embodiments monovalent antibody constructs provided herein lack the built-in avidity of bivalent antibodies, and would not spatially constrain two target antigens in the same manner.

Superior Efficacy/Bioactivity

As indicated herein, the monovalent antibody constructs described herein display superior efficacy and/or bioactivity as compared to the corresponding monospecific bivalent antibody construct. One non-limiting example of the efficacy and/or bioactivity of the monovalent antibody constructs according to the invention is represented by the ability of the monovalent antibody construct to inhibit growth of the target cell. In one embodiment, the superior efficacy and/or bioactivity of the monovalent antibody constructs is mainly a result of increased effector function of the monovalent antibody construct compared to the monospecific bivalent antibody construct. Examples of this type of monovalent antibody construct are represented by the monovalent lytic antibodies (MV-L).

ADCC

Increased effector functions include at least one of ADCC, ADCP, or CDC. Thus, in one embodiment, the monovalent antibody construct exhibits a higher degree of cell killing by ADCC than does the corresponding monospecific bivalent antibody construct. In accordance with this embodiment, the monovalent antibody construct exhibits an increase in ADCC activity of between about 1.2- to 1.6-fold over that of the corresponding monospecific bivalent antibody construct. In one embodiment, the monovalent antibody construct exhibits about a 1.3-fold increase in cell killing by ADCC than does the corresponding monospecific bivalent antibody construct. In one embodiment, the monovalent antibody construct exhibits about a 1.4-fold increase in cell killing by ADCC than does the corresponding monospecific bivalent antibody construct. In one embodiment, the monovalent antibody construct exhibits about a 1.5-fold increase in cell killing by ADCC than does the corresponding monospecific bivalent antibody construct.

In one embodiment, the monovalent antibody construct comprises an antigen-binding polypeptide construct that binds to HER2 and exhibits an increase in ADCC activity of between about 1.2- to 1.6-fold over that of the corresponding monospecific bivalent antibody construct. In one embodiment, the monovalent antibody construct comprises an antigen-binding polypeptide construct that binds to HER2 and exhibits about a 1.3-fold increase in cell killing by ADCC than does the corresponding monospecific bivalent antibody construct. In one embodiment, the monovalent antibody construct comprises an antigen-binding polypeptide construct that binds to HER2 and exhibits about a 1.5-fold increase in cell killing by ADCC than does the corresponding monospecific bivalent antibody construct.

ADCP

In one embodiment, the monovalent antibody construct exhibits a higher degree of cell killing by ADCP than does the corresponding monospecific bivalent antibody construct.

CDC

In one embodiment, the monovalent antibody construct exhibits a higher degree of cell killing by CDC than does the corresponding monospecific bivalent antibody construct. In one embodiment, the monovalent antibody construct comprises an antigen-binding polypeptide construct that binds to HER2 and exhibits about a 1.5-fold increase in cell killing by CDC than does the corresponding monospecific bivalent antibody construct.

In some embodiments is an isolated monovalent antibody construct described herein, wherein said construct possesses at least about 125% of at least one of the ADCC, ADCP and CDC of a corresponding bivalent antibody construct with two antigen binding polypeptide constructs. In some embodiments is an isolated monovalent antibody construct described herein, wherein said construct possesses at least about 150% of at least one of the ADCC, ADCP and CDC of a corresponding bivalent antibody construct with two antigen binding polypeptide constructs. In some embodiments is an isolated monovalent antibody construct described herein, wherein said construct possesses at least about 300% of at least one of the ADCC, ADCP and CDC of a corresponding bivalent antibody construct with two antigen binding polypeptide constructs.

Increased Binding Capacity to FcγRs

In some embodiments, the monovalent antibody constructs exhibit a higher binding capacity (Rmax) to one or more FcγRs. In one embodiment where the monovalent antibody construct comprises an antigen-binding polypeptide construct that binds to HER2, the monovalent antibody construct exhibits an increase in Rmax to one or more FcγRs over the corresponding monospecific bivalent antibody construct of between about 1.3- to 2-fold. In one embodiment where the monovalent antibody construct comprises an antigen-binding polypeptide construct that binds to HER2, the monovalent antibody construct exhibits an increase in Rmax to a CD16 FcγR of between about 1.3- to 1.8-fold over the corresponding monospecific bivalent antibody construct. In one embodiment where the monovalent antibody construct comprises an antigen-binding polypeptide construct that binds to HER2, the monovalent antibody construct exhibits an increase in Rmax to a CD32 FcγR of between about 1.3- to 1.8-fold over the corresponding monospecific bivalent antibody construct. In one embodiment where the monovalent antibody construct comprises an antigen-binding polypeptide construct that binds to HER2, the monovalent antibody construct exhibits an increase in Rmax to a CD64 FcγR of between about 1.3- to 1.8-fold over the corresponding monospecific bivalent antibody construct.

Increased Affinity for FcγRs

The monovalent antibody constructs provided herein have an unexpectedly increased affinity for FcγR as compared to corresponding bivalent antibody constructs. The increased Fc concentration resulting from the decoration is consistent with increased ADCC, ADCP, CDC activity.

In some embodiments, the monovalent antibody constructs exhibit an increased affinity for one or more FcγRs.

In one embodiment, where the monovalent antibody construct comprises an antigen-binding polypeptide construct that binds to HER2, the monovalent antibody constructs exhibit an increased affinity for at least one FcγR. In accordance with this embodiment, the monovalent antibody construct exhibits an increased affinity for CD32.

In another embodiment, is a monovalent antibody construct described herein that exhibits increased internalization compared to a corresponding monospecific bivalent antibody construct, thereby resulting in superior efficacy and/or bioactivity.

Pharmacokinetic Parameters

In certain embodiments, a monovalent antibody construct provided herein exhibits pharmacokinetic (PK) properties comparable with commercially available therapeutic antibodies. In one embodiment, the monovalent antibody constructs described herein exhibit PK properties similar to known therapeutic antibodies, with respect to serum concentration, t1/2, beta half-life, and/or CL. In one embodiment, the monovalent antibody constructs display in vivo stability comparable to or greater than said monospecific bivalent antibody construct. Such in vivo stability parameters include serum concentration, t1/2, beta half-life, and/or C_{L}.

In one embodiment, the monovalent antibody constructs provided herein show a higher volume of distribution (Vss) compared to the corresponding monospecific bivalent antibody constructs. Volume of distribution of an antibody relates to volume of plasma or blood (Vp), the volume of tissue (VT), and the tissue-to-plasma partitioning (kP). Under linear conditions, IgG antibodies are primarily distributed into the plasma compartment and the extravascular fluid following intravascular administration in animals or humans. In some embodiments, active transport processes such as uptake by neonatal Fc receptor (FcRn) also impact antibody biodistribution among other binding proteins.

In another embodiment, the monovalent antibody constructs according to the invention show a higher volume of distribution (Vss) and bind FcRn with similar affinity compared to the corresponding monospecific bivalent antibody constructs.

HER2 Binding Constructs

In some embodiments of the monovalent antibody construct described herein, the dimeric Fc polypeptide construct is heterodimeric. In one embodiment, the monovalent antibody construct described herein is designed to target a cell expressing HER2 and the antigen-binding polypeptide construct binds HER2. HER2 is proto-oncogene belonging to the human epidermal growth factor receptor (EGFR) family and is often overexpressed in a subset of breast cancers. The HER2 protein is also referred as the product of the neu gene, EGFR2, CD340, ErbB2 and p185. In some embodiments, the antigen-binding polypeptide construct binds HER2 and the target cell is a low, medium or high HER2 expressing cell. In an embodiment, the antigen-binding polypeptide construct binds HER2 and the target cell is a low HER2 expressing cell. In another embodiment, the antigen-binding polypeptide construct binds HER2 and the target cell is a low HER2 expressing cell with decreased binding to bivalent HER2 binding antibodies. In a further embodiment, the antigen-binding polypeptide construct binds HER2 and the target cell is a low HER2 expressing cell with decreased binding to trastuzumab. In an embodiment, the antigen-binding polypeptide construct binds HER2 and the target cell is a cancer cell. In a certain embodiment, the antigen-binding polypeptide construct binds HER2 and the target cell is a breast cancer cell.

In some embodiments of the monovalent antibody construct described herein, the dimeric Fc polypeptide construct is heterodimeric. In some embodiments of the monovalent antibody construct described, the antigen-binding polypeptide construct binds HER2. In some embodiments, the antigen-binding polypeptide construct binds at least one HER2 extracellular domain. In certain embodiments, the extracellular domain is at least one of ECD1, ECD2, ECD3 and ECD4. In certain embodiments, the antigen-binding polypeptide construct binds HER2 expressed by a target cell which is a low, medium or high HER2 expressing cell. In certain embodiments, the HER2 expressing cell displays decreased binding to bivalent HER2 binding antibodies. In an embodiment, the antigen-binding polypeptide construct binds HER2 and the target cell is at least one of an estrogen receptor negative cell, a progesterone receptor negative cell and anti-HER2 antibody resistant tumor cell with decreased binding to bivalent HER2 binding antibodies.

In some embodiments of the monovalent antibody construct described herein, the dimeric Fc polypeptide construct is heterodimeric. In certain embodiments of the monovalent antibody construct described herein, the monovalent antigen binding polypeptide construct is a Fab fragment, an scFv, and sdAb, an antigen binding peptide or a protein domain capable of binding the antigen. In some embodiments is provided an isolated monovalent antibody construct as described herein wherein the monovalent antigen binding polypeptide construct is a Fab fragment comprising a heavy chain polypeptide and a light chain polypeptide.

Provided herein is an isolated monovalent antibody construct that binds HER2 comprising: an antigen binding polypeptide construct which monovalently binds HER2; and a dimeric Fc polypeptide construct comprising two monomeric Fc polypeptides each comprising a CH3 domain, wherein one said monomeric Fc polypeptide is fused to at least one polypeptide from the antigen-binding polypeptide construct; wherein said antibody construct is anti-proliferative and is internalized by a target cell, wherein said construct displays an increase in binding density and Bmax (maximum binding) to HER2 displayed on the target cell as compared to a corresponding bivalent antibody construct which binds HER2, and wherein said construct displays at least one of higher ADCC, higher ADCP and higher CDC as compared to said corresponding bivalent HER2 binding antibody constructs.

Provided in certain embodiments is an isolated monovalent antibody construct that binds HER2 on a target cell with low HER2 expression, comprising: an antigen binding polypeptide construct which monovalently binds HER2; and a dimeric Fc polypeptide construct comprising two monomeric Fc polypeptides each comprising a CH3 domain, wherein one said monomeric Fc polypeptide is fused to at least one polypeptide from the antigen-binding polypeptide construct; wherein said antibody construct is anti-proliferative and is internalized by a target cell, wherein said construct displays an increase in binding density and Bmax (maximum binding) to HER2 displayed on the target cell as compared to a corresponding bivalent antibody construct which binds HER2, and wherein said construct displays at least one of higher ADCC, higher ADCP and higher CDC as compared to said corresponding bivalent HER2 binding antibody constructs. In certain embodiments, the target cell with low HER2 expression is a cancer cell. In some embodiments, the target cell with low HER2 expression is a breast cancer cell.

Provided herein is an isolated monovalent antibody construct that binds HER2 comprising: an antigen binding polypeptide construct which monovalently binds HER2 at an extracellular domain (ECD) which is at least one of ECD 1, ECD 2 and ECD 3-4; and a dimeric Fc polypeptide construct comprising two monomeric Fc polypeptides each comprising a CH3 domain, wherein one said monomeric Fc polypeptide is fused to at least one polypeptide from the antigen-binding polypeptide construct; wherein said antibody construct is anti-proliferative and is internalized by a target cell, wherein said construct displays an increase in binding density and Bmax (maximum binding) to at least one of HER2 ECD 1, 2, and 3-4 displayed on the target cell as compared to a corresponding bivalent antibody construct which binds HER2, and wherein said construct displays at least one of higher ADCC, higher ADCP and higher CDC as compared to said corresponding bivalent HER3 binding antibody constructs.

Provided herein is an isolated monovalent antibody construct that binds HER2 comprising: an antigen binding polypeptide construct which monovalently binds HER2 at an extracellular domain (ECD) which is at least one of ECD 1, ECD 2, ECD 3 and ECD4; and a dimeric Fc polypeptide construct comprising two monomeric Fc polypeptides each comprising a CH3 domain, wherein one said monomeric Fc polypeptide is fused to at least one polypeptide from the antigen-binding polypeptide construct; wherein said antibody construct is anti-proliferative and is internalized by a target cell, wherein said construct displays an increase in binding density and Bmax (maximum binding) to at least one of HER2 ECD 1, 2, 3 and 4 displayed on the target cell as compared to a corresponding bivalent antibody construct which binds HER2, and wherein said construct displays at least one of higher ADCC, higher ADCP and higher CDC as compared to said corresponding bivalent HER2 binding antibody constructs.

In an embodiment is the isolated monovalent antibody construct described herein, wherein the antibody construct inhibits target cell proliferation. In some embodiments is an isolated monovalent antibody construct described herein wherein said monovalent HER2 binding polypeptide construct is at least one of Fab, an scFv, an sdAb, or a polypeptide. In some embodiments is the isolated monovalent antibody construct described herein, wherein said construct possesses a higher degree of at least one of the ADCC, ADCP and CDC of a corresponding bivalent antibody construct with two antigen binding polypeptide construct. In some embodiments is the isolated monovalent antibody construct described herein, wherein said construct possesses at least about 105% of at least one of the ADCC, ADCP and CDC of a corresponding bivalent antibody construct with two antigen binding polypeptide construct. In some embodiments is an isolated monovalent antibody construct described herein, wherein said construct possesses greater than about 110% of at least one of the ADCC, ADCP and CDC of a corresponding bivalent antibody construct with two antigen binding polypeptide constructs.

Methods of Recombinant and Synthetic Production of Antibody Constructs:

Provided in certain embodiments is a method of producing a glycosylated monovalent antibody construct in stable mammalian cells, comprising: transfecting at least one stable mammalian cell with: a first DNA sequence encoding a first heavy chain polypeptide comprising a heavy chain variable domain and a first Fc domain polypeptide; a second DNA sequence encoding a second heavy chain polypeptide comprising a second Fc domain polypeptide, wherein said second heavy chain polypeptide is devoid of a variable domain; and a third DNA sequence encoding a light chain polypeptide comprising a light chain variable domain, such that the said first DNA sequence, said second DNA sequence and said third DNA sequences are transfected in said mammalian cell in a pre-determined ratio; translating the said first DNA sequence, said second DNA sequence, and said third DNA sequence in the at least one mammalian cell such that said heavy and light chain polypeptides are expressed as the desired glycosylated monovalent asymmetric antibody in said at least one stable mammalian cell. In some embodiments is the method of producing a glycosylated monovalent antibody construct in stable mammalian cells described herein, comprising transfecting at least two different cells with different pre-determined ratios of said first DNA sequence, said second DNA sequence and said third DNA sequence such that each of the two cells expresses the heavy chain polypeptides and the light chain polypeptide in a different ratio. In some embodiments is the method of producing a glycosylated monovalent antibody construct in stable mammalian cells described herein, comprising transfecting the at least one mammalian cell with a multi-cistronic vector comprising said first, second and third DNA sequence. In some embodiments, the at least one mammalian cell is selected from the group consisting of a VERO, HeLa, HEK, NSO, Chinese Hamster Ovary (CHO), W138, BHK, COS-7, Caco-2 and MDCK cell, and subclasses and variants thereof.

In some embodiments is the method of producing a glycosylated monovalent antibody construct in stable mammalian cells described herein wherein the predetermined ratio of the first DNA sequence:second DNA sequence:third DNA sequence is about 1:1:1. In some embodiments, the said predetermined ratio of the first DNA sequence:second DNA sequence:third DNA sequence is such that the amount of translated first heavy chain polypeptide is about equal to the amount of the second heavy chain polypeptide, and the amount of the light chain polypeptide.

In some embodiments is the method of producing a glycosylated monovalent antibody construct in stable mammalian cells described herein wherein the expression product of the at least one stable mammalian cell comprises a larger percentage of the desired glycosylated monovalent antibody as compared to the monomeric heavy or light chain polypeptides, or other antibodies.

In some embodiments is the method of producing a glycosylated monovalent antibody construct in stable mammalian cells described herein, said method comprising identifying and purifying the desired glycosylated monovalent antibody. In some embodiments, the said identification is by one or both of liquid chromatography and mass spectrometry.

Provided herein is a method of producing antibody constructs with improved ADCC comprising: transfecting at least one stable mammalian cell with: a first DNA sequence encoding a first heavy chain polypeptide comprising a heavy chain variable domain and a first Fc domain polypeptide; a second DNA sequence encoding a second heavy chain polypeptide comprising a second Fc domain polypeptide, wherein said second heavy chain polypeptide is devoid of a variable domain; and a third DNA sequence encoding a light chain polypeptide comprising a light chain variable domain, such that the said first DNA sequence, said second DNA sequence and said third DNA sequences are transfected in said mammalian cell in a pre-determined ratio; translating the said first DNA sequence, said second DNA sequence, and said third DNA sequence in the at least one mammalian cell such that said heavy and light chain polypeptides are expressed as a glycosylated monovalent antibody in said at least one stable mammalian cell, wherein said glycosylated monovalent asymmetric antibody has at least one of a higher ADCC, CDC and ADCP as compared to a corresponding wild-type antibody.

In certain embodiments are antibody constructs produced as recombinant molecules by secretion from yeast, a microorganism such as a bacterium, or a human or animal cell line. In embodiments, the polypeptides are secreted from the host cells.

Embodiments include a cell, such as a yeast cell transformed to express a heteromultimer protein described herein. In addition to the transformed host cells themselves, are provided culture of those cells, preferably a monoclonal (clonally homogeneous) culture, or a culture derived from a monoclonal culture, in a nutrient medium. If the polypeptide is secreted, the medium will contain the polypeptide, with the cells, or without the cells if they have been filtered or centrifuged away. Many expression systems are known and may be used, including bacteria (for example E. coli and Bacillus subtilis), yeasts (for example Saccharomyces cerevisiae, Kluyveromyces lactis and Pichia pastoris, filamentous fungi (for example Aspergillus), plant cells, animal cells and insect cells.

An antibody construct described herein is produced in conventional ways, for example from a coding sequence inserted in the host chromosome or on a free plasmid. The yeasts are transformed with a coding sequence for the desired protein in any of the usual ways, for example electroporation. Methods for transformation of yeast by electroporation are disclosed in Becker & Guarente (1990) Methods Enzymol. 194, 182.

Successfully transformed cells, i.e., cells that contain a DNA construct of the present invention, can be identified by well known techniques. For example, cells resulting from the introduction of an expression construct can be grown to produce the desired polypeptide. Cells can be harvested and lysed and their DNA content examined for the presence of the DNA using a method such as that described by Southern (1975) J. Mol. Biol. 98, 503 or Berent et al. (1985) Biotech. 3, 208. Alternatively, the presence of the protein in the supernatant can be detected using antibodies.

Useful yeast plasmid vectors include pRS403-406 and pRS413-416 and are generally available. Plasmids pRS403, pRS404, pRS405 and pRS406 are Yeast Integrating plasmids (YIps) and incorporate the yeast selectable markers HIS3, 7RP1, LEU2 and URA3. Plasmids pRS413-416 are Yeast Centromere plasmids (Ycps).

A variety of methods have been developed to operably link DNA to vectors via complementary cohesive termini. For instance, complementary homopolymer tracts can be added to the DNA segment to be inserted to the vector DNA. The vector and DNA segment are then joined by hydrogen bonding between the complementary honmopolymeric tails to form recombinant DNA molecules.

Synthetic linkers containing one or more restriction sites provide an alternative method of joining the DNA segment to vectors. The DNA segment, generated by endonuclease restriction digestion, is treated with bacteriophage T4 DNA polymerase or E. coli DNA polymerase 1, enzymes that remove protruding, . . . single-stranded termini with their 3′ 5′-exonucleolytic activities, and fill in recessed 3′-ends with their polymerizing activities.

The combination of these activities therefore generates blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces termini compatible with those of the DNA segment.

Synthetic linkers containing a variety of restriction endonuclease sites are commercially available from a number of sources.

Exemplary genera of yeast contemplated to be useful in the practice of the present invention as hosts for expressing the albumin, fusion proteins are Pichua (formerly classified as Hansenula), Saccharomyces, Kluyveromyces, Aspergillus, Candida, Torulopsis, Torulaspora, Schizosaccharomyces, Citeromyces, Pachysolen, Zygosaccharomyces, Debaromyces, Trichoderma, Cephalosporium, Humicola, Mucor, Neurospora, Yarrowia, Metschunikowia, Rhodosporidium, Leucosporidium, Botryoascus, Sporidiobolus, Endomycopsis, and the like. Preferred genera are those selected from the group consisting of Saccharomyces, Schizosaccharomyces, Kluyveromyces, Pichia and Torulaspora. Examples of Saccharomyces spp. are S. cerevisiae, S. italicus and S. rouxii.

Examples of Kluyveromyces spp. are K. fragilis, K. lactis and K. marxianus. A suitable Torulaspora species is T. delbrueckii. Examples of Pichia (Hansenula) spp. are P. angusta (formerly H. polymorpha), P. anomala (formerly H. anomala) and P. pastoris. Methods for the transformation of S. cerevisiae are taught generally in EP 251 744, EP 258 067 and WO 90/01063, all of which are incorporated herein by reference.

Provided are vectors containing a polynucleotide encoding an antibody construct protein described herein, host cells, and the production of the heteromultimer proteins by synthetic and recombinant techniques. The vector may be, for example, a phage, plasmid, viral, or retroviral vector. Retroviral vectors may be replication competent or replication defective. In the latter case, viral propagation generally will occur only in complementing host cells.

In certain embodiments, the polynucleotides encoding antibody constructs described herein are joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

In certain embodiments, the polynucleotide insert is operatively linked to an appropriate promoter, such as the phage lambda PL promoter, the E. coli lac, trp, phoA and rac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418, glutamine synthase, or neomycin resistance for eukaryotic cell culture, and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, NSO, 293, and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNH8A, pNH16a, pNH18A; pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Preferred expression vectors for use in yeast systems include, but are not limited to pYES2, pYD1, pTEF1/Zeo, pYES2/GS, pPICZ, pGAPZ, pGAPZalph, pPIC9, pPIC3.5, pHIL-D2, pHIL-S1, pPIC3.5K, pPIC9K, and PA0815 (all available from Invitrogen, Carlbad, Calif.). Other suitable vectors will be readily apparent to the skilled artisan.

In one embodiment, polynucleotides encoding antibody constructs described herein are fused to signal sequences that will direct the localization of a protein of the invention to particular compartments of a prokaryotic or eukaryotic cell and/or direct the secretion of a protein of the invention from a prokaryotic or eukaryotic cell. For example, in E. coli, one may wish to direct the expression of the protein to the periplasmic space. Examples of signal sequences or proteins (or fragments thereof) to which the antibody constructs are fused in order to direct the expression of the polypeptide to the periplasmic space of bacteria include, but are not limited to, the pelB signal sequence, the maltose binding protein (MBP) signal sequence, MBP, the ompA signal sequence, the signal sequence of the periplasmic E. coli heat-labile enterotoxin B-subunit, and the signal sequence of alkaline phosphatase. Several vectors are commercially available for the construction of fusion proteins which will direct the localization of a protein, such as the pMAL series of vectors (particularly the pMAL-.rho. series) available from New England Biolabs. In a specific embodiment, polynucleotides albumin fusion proteins of the invention may be fused to the pelB pectate lyase signal sequence to increase the efficiency of expression and purification of such polypeptides in Gram-negative bacteria. See, U.S. Pat. Nos. 5,576,195 and 5,846,818, the contents of which are herein incorporated by reference in their entireties.

Examples of signal peptides that are fused to antibody constructs in order to direct its secretion in mammalian cells include, but are not limited to, the MPIF-1 signal sequence (e.g., amino acids 1-21 of GenBank Accession number AAB51134), the stanniocalcin signal sequence (MLQNSAVLLLLVISASA), and a consensus signal sequence (MPTWAWWLFLVLLLALWAPARG). A suitable signal sequence that may be used in conjunction with baculoviral expression systems is the gp67 signal sequence (e.g., amino acids 1-19 of GenBank Accession Number AAA72759).

Vectors which use glutamine synthase (GS) or DHFR as the selectable markers can be amplified in the presence of the drugs methionine sulphoximine or methotrexate, respectively. An advantage of glutamine synthase based vectors are the availability of cell lines (e.g., the murine myeloma cell line, NSO) which are glutamine synthase negative. Glutamine synthase expression systems can also function in glutamine synthase expressing cells (e.g., Chinese Hamster Ovary (CHO) cells) by providing additional inhibitor to prevent the functioning of the endogenous gene. A glutamine synthase expression system and components thereof are detailed in PCT publications: WO87/04462; WO86/05807; WO89/10036; WO89/10404; and WO91/06657, which are hereby incorporated in their entireties by reference herein. Additionally, glutamine synthase expression vectors can be obtained from Lonza Biologics, Inc. (Portsmouth, N.H.). Expression and production of monoclonal antibodies using a GS expression system in murine myeloma cells is described in Bebbington et al., Bio/technology 10:169(1992) and in Biblia and Robinson Biotechnol. Prog. 11:1(1995) which are herein incorporated by reference.

Provided herein is a host cell comprising nucleic acid encoding an isolated monovalent antibody construct described herein. In certain embodiments is the host cell described herein wherein the nucleic acid encoding the antigen binding polypeptide construct and the nucleic acid encoding the Fc construct are present in a single vector.

Provided herein is a method of preparing the isolated monovalent antibody construct described herein, the method comprising the steps of: (a) culturing a host cell comprising nucleic acid encoding the antibody construct; and (b) recovering the antibody construct from the host cell culture.

Also provided are host cells containing vector constructs described herein, and additionally host cells containing nucleotide sequences that are operably associated with one or more heterologous control regions (e.g., promoter and/or enhancer) using techniques known of in the art. The host cell can be a higher eukaryotic cell, such as a mammalian cell (e.g., a human derived cell), or a lower eukaryotic cell, such as a yeast cell, or the host cell can be a prokaryotic cell, such as a bacterial cell. A host strain may be chosen which modulates the expression of the inserted gene sequences, or modifies and processes the gene product in the specific fashion desired. Expression from certain promoters can be elevated in the presence of certain inducers; thus expression of the genetically engineered polypeptide may be controlled. Furthermore, different host cells have characteristics and specific mechanisms for the translational and post-translational processing and modification (e.g., phosphorylation, cleavage) of proteins. Appropriate cell lines can be chosen to ensure the desired modifications and processing of the foreign protein expressed.

Introduction of the nucleic acids and nucleic acid constructs of the invention into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that the polypeptides of the present invention may in fact be expressed by a host cell lacking a recombinant vector.

In addition to encompassing host cells containing the vector constructs discussed herein, the invention also encompasses primary, secondary, and immortalized host cells of vertebrate origin, particularly mammalian origin, that have been engineered to delete or replace endogenous genetic material, and/or to include genetic material. The genetic material operably associated with the endogenous polynucleotide may activate, alter, and/or amplify endogenous polynucleotides.

In addition, techniques known in the art may be used to operably associate heterologous polynucleotides and/or heterologous control regions (e.g., promoter and/or enhancer) with endogenous polynucleotide sequences encoding a Therapeutic protein via homologous recombination (see, e.g., U.S. Pat. No. 5,641,670, issued Jun. 24, 1997; International Publication Number WO 96/29411; International Publication Number WO 94/12650; Koller et al., Proc. Natl. Acad. Sci. USA 86:8932-8935 (1989); and Zijlstra et al., Nature 342:435-438 (1989), the disclosures of each of which are incorporated by reference in their entireties).

Antibody constructs described herein can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, hydrophobic charge interaction chromatography and lectin chromatography. Most preferably, high performance liquid chromatography (“HPLC”) is employed for purification.

In certain embodiments the heteromultimer proteins of the invention are purified using Anion Exchange Chromatography including, but not limited to, chromatography on Q-sepharose, DEAE sepharose, poros HQ, poros DEAF, Toyopearl Q, Toyopearl QAE, Toyopearl DEAE, Resource/Source Q and DEAE, Fractogel Q and DEAE columns.

In specific embodiments the proteins described herein are purified using Cation Exchange Chromatography including, but not limited to, SP-sepharose, CM sepharose, poros HS, poros CM, Toyopearl SP, Toyopearl CM, Resource/Source S and CM, Fractogel S and CM columns and their equivalents and comparables.

In addition, antibody constructs described herein can be chemically synthesized using techniques known in the art (e.g., see Creighton, 1983, Proteins: Structures and Molecular Principles, W. H. Freeman & Co., N.Y and Hunkapiller et al., Nature, 310:105-111 (1984)). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4diaminobutyric acid, alpha-amino isobutyric acid, 4aminobutyric acid, Abu, 2-amino butyric acid, g-Abu, e-Ahx, 6amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general. Furthermore, the amino acid can be D (dextrorotary) or L (levorotary).

Testing of the Monovalent Antibody Constructs. FcγR, FcRn and C1q Binding

The monovalent antibody constructs according to the invention exhibit enhanced effector function compared to the corresponding monospecific bivalent antibody construct. The effector functions of the monovalent antibody constructs can be tested as follows. In vitro and/or in vivo cytotoxicity assays can be conducted to assess ADCP, CDC and/or ADCC activities. For example, Fc receptor (FcR) binding assays can be conducted to measure FcγR binding. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). An example of an in vitro assay to assess ADCC activity of a molecule of interest is described in U.S. Pat. No. 5,500,362 or 5,821,337. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998). C1q binding assays may also be carried out to determine if the monovalent antibody constructs are capable of binding C1q and hence activating CDC. To assess complement activation, a CDC assay, e.g. as described in Gazzano-Santoro et al., J. Immunol. Methods 202:163 (1996), may be performed. FcRn binding such as by SPR and in vivo PK determinations of antibodies can also be performed using methods well known in the art.

Biological and Therapeutic Uses:

In certain embodiments, constructs described herein, are used in assays to test for one or more biological activities. If a construct exhibits an activity in a particular assay, it is likely that the antigen binding construct comprised by the antibody construct is implicated in the diseases associated with the biological activity. Thus, the construct is of use in a treatment of the associated disease.

In certain embodiments is use of a monovalent antibody construct described herein for the manufacture of a medicament for inhibiting multimerization of an antigen molecule. In certain embodiments is use of a monovalent antibody construct for inhibiting binding of an antigen to its cognate binding partner.

In certain embodiments, provided is a method of treating a disease or disorder comprising administering to a patient in which such treatment, prevention or amelioration is desired, an antibody construct described herein, in an amount effective to treat, prevent or ameliorate the disease or disorder.

In certain embodiments, antibody constructs described herein are used in the diagnosis, prognosis, prevention and/or treatment of diseases and/or disorders of the endocrine system. In some embodiments, antibody constructs described herein are used in the diagnosis, prognosis, prevention and/or treatment of diseases and/or disorders of the nervous system.

In certain embodiments, antibody constructs described herein are used in the diagnosis, prognosis, prevention and/or treatment of diseases and/or disorders of the immune system. In certain embodiments, antibody constructs described herein are used in the diagnosis, prognosis, prevention and/or treatment of diseases and/or disorders of the respiratory system.

In certain embodiments, antibody constructs described herein are used in the diagnosis, prognosis, prevention and/or treatment of diseases and/or disorders of the cardiovascular system. In some embodiments, antibody constructs described herein are used in the diagnosis, prognosis, prevention and/or treatment of diseases and/or disorders of the reproductive system.

In certain embodiments, antibody constructs described herein are used in the diagnosis, prognosis, prevention and/or treatment of diseases and/or disorders of the digestive system. In certain embodiments, antibody constructs described herein are used in the diagnosis, prognosis, prevention and/or treatment of diseases or disorders relating to the blood.

In some embodiments, antibody constructs described herein and/or polynucleotides encoding the antibody constructs described herein, are used in the diagnosis, detection and/or treatment of diseases and/or disorders associated with activities that include, but are not limited to, prohormone activation, neurotransmitter activity, cellular signaling, cellular proliferation, cellular differentiation, and cell migration.

In an aspect, antibody constructs described herein are directed to antibody-based therapies which involve administering antibody constructs, to a patient for treating one or more of the disclosed diseases, disorders, or conditions. Therapeutic compounds described herein include, but are not limited to, antibody constructs described herein, nucleic acids encoding antibody constructs described herein.

In a specific embodiment, are antibody-based therapies which involve administering antibody constructs described herein comprising at least a fragment or variant of an antibody to a patient for treating one or more diseases, disorders, or conditions, including but not limited to: neural disorders, immune system disorders, muscular disorders, reproductive disorders, gastrointestinal disorders, pulmonary disorders, cardiovascular disorders, renal disorders, proliferative disorders, and/or cancerous diseases and conditions, and/or as described elsewhere herein.

The antibody constructs described herein, comprising at least a fragment or variant of an antibody may be administered alone or in combination with other types of treatments (e.g., radiation therapy, chemotherapy, hormonal therapy, immunotherapy and anti-tumor agents). Generally, administration of products of a species origin or species reactivity (in the case of antibodies) that is the same species as that of the patient is preferred. Thus, in an embodiment, human antibodies, fragments derivatives, analogs, or nucleic acids, are administered to a human patient for therapy or prophylaxis.

Also provided is a method of treating an infectious disease in a patient, said method comprising administering to the patient a therapeutically effective amount of a monovalent antibody construct described herein. In certain embodiments, the infectious disease is caused by a virtual agent. In certain embodiments, the infectious disease is caused by bacterial agent or a fungal agent. Bacterial agents that can be treated by providing an amount of a monovalent antibody construct described herein include and are not limited to: Corynebacterium diphtheriae, Streptococcus pneumoniae, Neisseria meningitides, E. Coli, streptococcus, Clostridium tetani, C. difficile, Mycobacterium tuberculosis, C. parvum, vancomycin-resistant enterococcus, methicillin-resistant S. aureus and others. Viral agents that can be treated by providing an amount of a monovalent antibody construct described herein include, but are not limited to: Haemophilus influenzae, group A, cytomegalovirus (CMV), respiratory syncytial virus (RSV), hepatitis A virus (HAV), hepatitis B virus (HBV), rabies, vaccinia, vesicular stomatitis virus (VZV), HIV, WNV, SARs. Fungal agents that can be treated by providing an amount of a monovalent antibody construct described herein include, but are not limited to: cryptococcal meningitis, C. neoformans (CN), Histoplasma capsulatum (HC).

Provided is a kit for detecting the presence of a biomarker of interest in an individual, said kit comprising (a) an isolated monovalent antibody construct described herein; and (b) instructions for use. In certain embodiments are kits for the detection of at least one of HER2 and a soluble ECD thereof, said kit comprising (a) an isolated monovalent HER2 binding antibody construct described herein; and (b) instructions for use. In some embodiments is a kit for determining concentration of at least one of HER2 and a soluble ECD thereof, said kit comprising (a) an isolated monovalent HER2 binding antibody construct described herein; and (b) instructions for use.

Treatment of Cancers

Provided herein is the use of a monovalent antibody construct described herein for the manufacture of a medicament for treating cancer. Also provided is use of a monovalent antibody construct described herein for the manufacture of a medicament for an immune system disorder. In certain embodiments is use of a monovalent antibody construct described herein for the manufacture of a medicament for inhibiting growth of a tumor. In certain embodiments is use of a monovalent antibody construct described herein for the manufacture of a medicament for shrinking a tumor.

Provided herein is the use of a monovalent HER2 binding antibody construct described herein for the manufacture of a medicament for treating cancer. In certain embodiments, the cancer is a low-HER2 expressing cancer. In certain embodiments, the cancer is resistant to treatment with a bivalent HER2 antibody. Provided herein is the use of a monovalent HER2 binding antibody construct described herein for the manufacture of a medicament for treating cancers resistant to treatment with Trastazaumab.

In one embodiment, the monovalent antibody constructs described herein are used in the treatment of cancer. In one embodiment, monovalent antibody constructs comprising an HER2 binding polypeptide construct described herein are useful in the treatment of a cancer or any proliferative disease associated with HER dysfunction, including HER1 dysfunction, HER2 dysfunction, HER 3 dysfunction, and/or HER4 dysfunction. In certain embodiments the cancer is at least one of breast cancer, gastric cancer, brain cancer, lung cancer or is at least one type of carcinoma.

In one embodiment, HER2 binding monovalent antibody constructs described herein are used in the treatment of a breast cancer cell. In certain embodiments, the HER2 binding monovalent antibody constructs are used in the preparation of a pharmaceutical composition for administration to an individual suffering from breast cancer. In some embodiments is the treatment of breast cancer in an individual by providing to said individual an effective amount of at least one HER2 binding monovalent antibody construct described herein.

In one embodiment, a HER2 binding monovalent antibody construct described herein is used to treat patients that are partially responsive to current anti-HER2 therapies. In one embodiment, HER2 binding monovalent antibody constructs described herein are used to treat patients that are resistant to current anti-HER2 therapies. In another embodiment, HER2 binding monovalent antibody constructs described herein are used to treat patients that are developing resistance to current anti-HER2 therapies.

In one embodiment, HER2 binding monovalent antibody constructs described herein are useful to treat patients that are unresponsive to current anti-HER2 therapies. In certain embodiments, these patients suffer from a triple negative cancer. In some embodiments, the triple-negative cancer is a breast cancer with low to negligent expression of the genes for estrogen receptor (ER), progesterone receptor (PR) and Her2. In certain other embodiments the HER2 binding monovalent antibody constructs described herein are provided to patients that are unresponsive to current anti-HER2 therapies, optionally in combination with one or more current anti-HER2 therapies. In some embodiments the current anti-HER2 therapies include, but are not limited to, anti-HER2 or anti-HER3 monospecific bivalent antibodies, trastuzumab, pertuzumab, T-DM1, a bi-specific HER2/HER3 scFv, or combinations thereof. In one embodiment, a monovalent antibody construct described herein is used to treat patients that are not responsive to trastuzumab, pertuzumab, T-DM1, anti-HER2, or anti-HER3, alone or in combination.

In one embodiment, a HER2 binding monovalent antibody construct that comprise an antigen-binding polypeptide construct that binds HER2 can be used in the treatment of patients with metastatic breast cancer. In one embodiment, a HER2 binding monovalent antibody is useful in the treatment of patients with locally advanced or advanced metastatic breast cancer. In one embodiment, a HER2 binding monovalent antibody is useful in the treatment of patients with refractory breast cancer. In one embodiment, a HER2 binding monovalent antibody is provided to a patient for the treatment of metastatic breast cancer when said patient has progressed on previous anti-HER2 therapy. In one embodiment, a HER2 binding monovalent antibody described herein can be used in the treatment of patients with triple negative breast cancers. In one embodiment, a HER2 binding monovalent antibody described herein is used in the treatment of patients with advanced, refractory HER2-amplified, heregulin positive cancers.

Provided are HER2 binding monovalent antibody constructs to be administered in combination with other known therapies for the treatment of cancer. In accordance with this embodiment, the monovalent antibody constructs can be administered in combination with other monovalent antibody constructs or multivalent antibodies with non-overlapping binding target epitopes to significantly increase the Bₘₐₓ and antibody dependent cytotoxic activity above FSAs. For example, a monovalent anti-HER2 antibody according to the invention can be administered in combination as follows: 1) a monovalent antibody construct such as OA1-Fab-Her2 (based on herceptin) in combination with OA5-Fab-Her2 (based on pertuzumab); 2) ° A1-Fab-Her2 and/or OA5-Fab-Her2 in combination with cetuximab bivalent EGFR antibody; and 3) multiple combinations of non-competing antibodies directed at the same and different surface antigens on the same target cell. In certain embodiments, the monovalent antibody constructs described herein are administered in combination with a therapy selected from Herceptin™, TDM1, afucosylated antibodies or Perjeta for the treatment of patients with advanced HER2 amplified, heregulin-positive breast cancer. In a certain embodiment, a monovalent antibody construct described herein is administered in combination with Herceptin™ or Perjeta in patients with HER2-expressing carcinomas of the distal esophagus, gastroesophageal (GE) junction and stomach.

Gene Therapy:

In a specific embodiment, nucleic acids comprising sequences encoding antibody constructs described herein are administered to treat, inhibit or prevent a disease or disorder associated with aberrant expression and/or activity of a protein, by way of gene therapy. Gene therapy refers to therapy performed by the administration to a subject of an expressed or expressible nucleic acid. In this embodiment of the invention, the nucleic acids produce their encoded protein that mediates a therapeutic effect. Any of the methods for gene therapy available in the art can be used.

Therapeutic/Prophylactic Administration and Composition

Provided are methods of treatment, inhibition and prophylaxis by administration to a subject of an effective amount of an antibody construct or pharmaceutical composition described herein. In an embodiment, the antibody constructs is substantially purified (e.g., substantially free from substances that limit its effect or produce undesired side-effects). In certain embodiments, the subject is an animal, including but not limited to animals such as cows, pigs, horses, chickens, cats, dogs, etc., and in certain embodiments, a mammal, and most preferably human.

Various delivery systems are known and can be used to administer an antibody construct formulation described herein, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, in certain embodiments, it is desirable to introduce the antibody construct compositions described herein into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it is desirable to administer the antibody constructs, or compositions described herein locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the antibody constructs or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the antibody constructs or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Ha. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, e.g., the brain, thus requiring only a fraction of the systemic dose (see, e.g., Goodson, in Medical Applications of Controlled Release, supra, vol. 2, pp. 115-138 (1984)).

In a specific embodiment comprising a nucleic acid encoding antibody constructs described herein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

In certain embodiments a one arm monovalent antibody construct described herein is administered as a combination with other one arm monovalent or multivalent antibodies with non-overlapping binding target epitopes.

Also provided herein are pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In certain embodiments, the composition comprising the antibody constructs is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In certain embodiments, the compositions described herein are formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxide isopropylamine, triethylamine, 2-ethylamino ethanol, histidine, procaine, etc.

The amount of the composition described herein which will be effective in the treatment, inhibition and prevention of a disease or disorder associated with aberrant expression and/or activity of a Therapeutic protein can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses are extrapolated from dose-response curves derived from in vitro or animal model test systems.

Conjugation with a Drug Molecule:

In certain embodiments is a pharmaceutical composition comprising the monovalent antibody construct described herein conjugated to a drug molecule. In certain embodiments, at least one drug molecule is a therapeutic agent. In certain embodiments, the drug molecule is a toxin. In certain embodiments, the drug molecule is an antigen analog. In an embodiment, the drug molecule is a natural product, analog, or prodrug thereof.

In certain embodiment, the drug molecule is a biomolecule. In an embodiment, the drug molecule is a natural or synthetic nucleic acid. In some embodiments, at least one drug molecule is one or more of a DNA, PNA, and/or RNA oligomer.

Demonstration of Therapeutic or Prophylactic Activity:

The antibody constructs or pharmaceutical compositions described herein are tested in vitro, and then in vivo for the desired therapeutic or prophylactic activity, prior to use in humans. For example, in vitro assays to demonstrate the therapeutic or prophylactic utility of a compound or pharmaceutical composition include, the effect of a compound on a cell line or a patient tissue sample. The effect of the compound or composition on the cell line and/or tissue sample can be determined utilizing techniques known to those of skill in the art including, but not limited to, rosette formation assays and cell lysis assays. In accordance with the invention, in vitro assays which can be used to determine whether administration of a specific compound is indicated, include in vitro cell culture assays in which a patient tissue sample is grown in culture, and exposed to or otherwise administered antibody construct, and the effect of such antibody construct upon the tissue sample is observed.

Provided are antibody constructs which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH₄; acetylation, formylation, oxidation, reduction; metabolic synthesis in the presence of tunicamycin; etc.

Additional post-translational modifications encompassed herein include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The antibody constructs are modified with a detectable label, such as an enzymatic, fluorescent, isotopic or affinity label to allow for detection and isolation of the protein.

Examples of suitable enzymes include horseradish peroxidase, alkaline phosphatase, beta-galactosidase, or acetylcholinesterase; examples of suitable prosthetic group complexes include streptavidin biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include iodine, carbon, sulfur, tritium, indium, technetium, thallium, gallium, palladium, molybdenum, xenon, fluorine.

In specific embodiments, antibody constructs or fragments or variants thereof are attached to macrocyclic chelators that associate with radiometal ions.

As mentioned, the antibody constructs described herein are modified by either natural processes, such as post-translational processing, or by chemical modification techniques which are well known in the art. It will be appreciated that the same type of modification may be present in the same or varying degrees at several sites in a given polypeptide. Polypeptides of the invention may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POST-TRANSLATIONAL COVALENT MODIFICATION OF PROTEINS, B. C. Johnson, Ed., Academic Press, New York, pgs. 1-12 (1983); Seifter et al., Meth. Enzymol. 182:626-646 (1990); Rattan et al., Ann. N.Y. Acad. Sci. 663:48-62 (1992)).

In certain embodiments, antibody constructs may also be attached to solid supports, which are particularly useful for immunoassays or purification of polypeptides that are bound by, that bind to, or associate with albumin fusion proteins of the invention. Such solid supports include, but are not limited to, glass, cellulose, polyacrylamide, nylon, polystyrene, polyvinyl chloride or polypropylene.

Also provided herein are chemically modified derivatives of the antibody constructs which may provide additional advantages such as increased solubility, stability and circulating time of the polypeptide, or decreased immunogenicity (see U.S. Pat. No. 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The proteins may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a Therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 105,500, 16,000, 16,500, 17,000, 17,500, 18,000, 18,500, 19,000, 19,500, 20,000, 25,000, 30,000, 35,000, 40,000, 45,000, 50,000, 55,000, 60,000, 65,000, 70,000, 75,000, 80,000, 85,000, 90,000, 95,000, or 100,000 kDa.

The presence and quantity of antibody constructs described herein may be determined using ELISA, a well known immunoassay known in the art. In one ELISA protocol that would be useful for detecting/quantifying heteromultimers described herein, comprises the steps of coating an ELISA plate with an anti-human serum albumin antibody, blocking the plate to prevent non-specific binding, washing the ELISA plate, adding a solution containing the protein described herein (at one or more different concentrations), adding a secondary anti-antibody construct polypeptide specific antibody coupled to a detectable label (as described herein or otherwise known in the art), and detecting the presence of the secondary antibody

In certain embodiments is a pharmaceutical composition comprising the monovalent antibody construct described herein and an adjuvant. In certain embodiments is the pharmaceutical composition described herein, further comprising a drug molecule conjugated to the monovalent antibody construct. In certain embodiments, the drug molecule is for the treatment of an autoimmune disorder. In some embodiments, the drug molecule is for the treatment of a cancer. In some embodiments, the drug molecule is a chemotherapeutic agent.

Provided herein is a method of treating cancer comprising providing to a patient in need thereof an effective amount of a pharmaceutical composition described herein. In one embodiment, the cancer to be treated is breast cancer. In another embodiment, the cancer to be treated is a breast cancer, wherein the cells of the breast cancer express HER2 protein in high, medium, or low density. HER2 belongs to the EGFR family of receptors and tends to be overexpressed in a subset of breast cancers. The HER2 protein is also referred as the product of the neu gene, EGFR2, CD340, ErbB2 and p185. The following Table A describes the expression level of HER2 on several representative breast cancer cell lines (Subik et al. (2010) Breast Cancer: Basic Clinical Research:4; 35-41; Prang et a. (2005) British Journal of Cancer Research:92; 342-349). As shown in the table, MCF-7 and MDA-MB-231 cells are considered to be low HER2 expressing cells; SKOV3 cells are considered to be medium HER2 expressing cells, and SKBR3 cells are considered to be high HER2 expressing cells.

TABLE A2

| Cell Line | HER2 level | HER2 Bmax (×10³) |
|---|---|---|
| MCF-7 | 0-1+ | 25 |
| MDA-MB-231 | 0-1+ | 14 (triple negative) |
| SKOV3 | 2+ | 300 |
| SKBr3 | 3+ | 976 |

In some embodiments is a method of treating an immune system disorder comprising providing to a patient in need thereof an effective amount of a pharmaceutical composition described herein. In certain embodiments is a method of inhibiting growth of a tumor, comprising contacting the tumor with a composition comprising an effective amount of a monovalent antibody construct described herein. Provided is a method of shrinking a tumor, comprising contacting the tumor with a composition comprising an effective amount of a monovalent antibody construct described herein. In some embodiments is a method of inhibiting multimerization of an antigen molecule, comprising contacting the antigen with a composition comprising an effective amount of a monovalent antibody construct described herein. Provided herein is a method of inhibiting binding of an antigen to its cognate binding partner comprising contacting the antigen with a composition comprising an amount of a monovalent antibody construct sufficient to bind to the antigen.

Provided in certain embodiments is a method of producing a glycosylated monovalent antibody construct in stable mammalian cells, comprising: transfecting at least one stable mammalian cell with: a first DNA sequence encoding a first heavy chain polypeptide comprising a heavy chain variable domain and a first Fc domain polypeptide; a second DNA sequence encoding a second heavy chain polypeptide comprising a second Fc domain polypeptide, wherein said second heavy chain polypeptide is devoid of a variable domain; and a third DNA sequence encoding a light chain polypeptide comprising a light chain variable domain, such that the said first DNA sequence, said second DNA sequence and said third DNA sequences are transfected in said mammalian cell in a pre-determined ratio; translating the said first DNA sequence, said second DNA sequence, and said third DNA sequence in the at least one mammalian cell such that said heavy and light chain polypeptides are expressed as the desired glycosylated monovalent asymmetric antibody in said at least one stable mammalian cell. In some embodiments is the method of producing a glycosylated monovalent antibody construct in stable mammalian cells described herein, comprising transfecting at least two different cells with different pre-determined ratios of said first DNA sequence, said second DNA sequence and said third DNA sequence such that each of the two cells expresses the heavy chain polypeptides and the light chain polypeptide in a different ratio. In some embodiments is the method of producing a glycosylated monovalent antibody construct in stable mammalian cells described herein, comprising transfecting the at least one mammalian cell with a multi-cistrionic vector comprising said first, second and third DNA sequence. In some embodiments, the at least one mammalian cell is selected from the group consisting of a VERO, HeLa, HEK, NSO, Chinese Hamster Ovary (CHO), W138, BHK, COS-7, Caco-2 and MDCK cell, and subclasses and variants thereof.

In some embodiments is the method of producing a glycosylated monovalent antibody construct in stable mammalian cells described herein wherein the predetermined ratio of the first DNA sequence:second DNA sequence:third DNA sequence is about 1:1:1. In some embodiments, the said predetermined ratio of the first DNA sequence:second DNA sequence:third DNA sequence is such that the amount of translated first heavy chain polypeptide is about equal to the amount of the second heavy chain polypeptide, and the amount of the light chain polypeptide.

In some embodiments is the method of producing a glycosylated monovalent antibody construct in stable mammalian cells described herein wherein the expression product of the at least one stable mammalian cell comprises a larger percentage of the desired glycosylated monovalent antibody as compared to the monomeric heavy or light chain polypeptides, or other antibodies.

In some embodiments is the method of producing a glycosylated monovalent antibody construct in stable mammalian cells described herein, said method comprising identifying and purifying the desired glycosylated monovalent antibody. In some embodiments, the said identification is by one or both of liquid chromatography and mass spectrometry.

Provided herein is a method of producing antibody constructs with improved ADCC comprising: transfecting at least one stable mammalian cell with: a first DNA sequence encoding a first heavy chain polypeptide comprising a heavy chain variable domain and a first Fc domain polypeptide; a second DNA sequence encoding a second heavy chain polypeptide comprising a second Fc domain polypeptide, wherein said second heavy chain polypeptide is devoid of a variable domain; and a third DNA sequence encoding a light chain polypeptide comprising a light chain variable domain, such that the said first DNA sequence, said second DNA sequence and said third DNA sequences are transfected in said mammalian cell in a pre-determined ratio; translating the said first DNA sequence, said second DNA sequence, and said third DNA sequence in the at least one mammalian cell such that said heavy and light chain polypeptides are expressed as a glycosylated monovalent antibody in said at least one stable mammalian cell, wherein said glycosylated monovalent asymmetric antibody has a higher ADCC as compared to a corresponding wild-type antibody.

Provided herein is a method of increasing antibody concentration in at least one target cell comprising providing to the target cell a monovalent antibody construct comprising: an antigen-binding polypeptide construct which monovalently binds an antigen; a dimeric Fc region; wherein said monovalent antibody construct displays an increase in binding density and Bmax (maximum binding) to a target cell displaying said antigen as compared to a corresponding bivalent antibody construct with two antigen binding regions, and wherein said monovalent antibody construct shows better therapeutic efficacy compared to a corresponding bivalent antibody construct, and wherein said efficacy is not caused by crosslinking of the antigen, antigen dimerization, prevention of antigen modulation, or prevention of antigen activation.

Provided herein are isolated monovalent antibody constructs comprising an antigen-binding polypeptide construct which monovalently binds an antigen; and a dimeric Fc polypeptide construct comprising a CH3 domain; wherein said monovalent antibody construct displays an increase in binding density and Bmax (maximum binding) to a target cell displaying said antigen as compared to a corresponding bivalent antibody construct with two antigen binding regions, and wherein said monovalent antibody construct shows better therapeutic efficacy compared to a corresponding bivalent antibody construct, and wherein said efficacy is not caused by crosslinking of the antigen, antigen dimerization, prevention of antigen modulation, or prevention of antigen activation.

Provided herein are isolated monovalent antibody construct that binds HER2 comprising: an antigen binding polypeptide construct which monovalently binds HER2; and a dimeric Fc polypeptide construct comprising a CH3 domain; wherein said antibody construct is internalized by a target cell, wherein said construct displays an increase in binding density and Bmax (maximum binding) to HER2 displayed on the target cell as compared to a corresponding bivalent antibody construct which bivalently binds HER2, and wherein said construct displays at least one of higher ADCC, higher ADCP and higher CDC as compared to said corresponding bivalent HER2 binding antibody constructs.

Provided herein is a method of producing a glycosylated monovalent antibody construct in stable mammalian cells, comprising: transfecting at least one stable mammalian cell with: a first DNA sequence encoding a first heavy chain polypeptide comprising a heavy chain variable domain and a first Fc domain polypeptide; a second DNA sequence encoding a second heavy chain polypeptide comprising a second Fc domain polypeptide, wherein said second heavy chain polypeptide is devoid of a variable domain; and a third DNA sequence encoding a light chain polypeptide comprising a light chain variable domain, such that the said first DNA sequence, said second DNA sequence and said third DNA sequences are transfected in said mammalian cell in a pre-determined ratio; translating the said first DNA sequence, said second DNA sequence, and said third DNA sequence in the at least one mammalian cell such that said heavy and light chain polypeptides are expressed as the desired glycosylated monovalent asymmetric antibody in said at least one stable mammalian cell.

Provided is a kit for detecting the presence of a biomarker of interest in an individual, said kit comprising (a) an isolated monovalent antibody construct described herein; and (b) instructions for use.

Also provided are transgenic organisms modified to contain nucleic acid molecules described herein to encode and express monovalent antibody constructs described herein.

Provided in certain embodiments is an isolated monovalent antibody construct that binds HER2 on a target cell with low HER2 expression, comprising: an antigen binding polypeptide construct which monovalently binds HER2; and a dimeric Fc polypeptide construct comprising two monomeric Fc polypeptides each comprising a CH3 domain, wherein one said monomeric Fc polypeptide is fused to at least one polypeptide from the antigen-binding polypeptide construct; wherein said antibody construct is anti-proliferative and is internalized by a target cell, wherein said construct displays an increase in binding density and Bmax (maximum binding) to HER2 displayed on the target cell as compared to a corresponding bivalent antibody construct which binds HER2, and wherein said construct displays at least one of higher ADCC, higher ADCP and higher CDC as compared to said corresponding bivalent HER2 binding antibody constructs. In certain embodiments, the target cell with low HER2 expression is a cancer cell. In some embodiments, the target cell with low HER2 expression is a breast cancer cell.

Also provided is a method of preventing antigen extra-cellular domain proteolytic cleavage by binding of the antigen to a monovalent antibody construct provided herein.

Various publications are cited herein, the disclosures of which are incorporated by reference in their entireties.

### REFERENCES

Bowles J A, Wang S Y, Link B K, Allan B, Beuerlein G, Campbell M A, Marquis D, Ondek B, Wooldridge J E, Smith B J, Breitmeyer J B, Weiner G J. Anti-CD20 monoclonal antibody with enhanced affinity for CD16 activates N K cells at lower concentrations and more effectively than rituximab. Blood. 2006 Oct. 15; 108(8):2648-54. Epub 2006 Jul. 6.
Desjarlais J R, Lazar G A. Modulation of antibody effector function. Exp Cell Res. 2011 May 15; 317(9):1278-85.
Ferrara C, Grau S, Jäger C, Sondermann P, Brünker P, Waldhauer I, Hennig M, Ruf A, Rufer A C, Stihle M, Umaña P, Benz J. Unique carbohydrate-carbohydrate interactions are required for high affinity binding between FcgammaRIII and antibodies lacking core fucose. Proc Natl Acad Sci USA. 2011 Aug. 2; 108(31):12669-74.
Heider K H, Kiefer K, Zenz T, Volden M, Stilgenbauer S, Ostermann E, Baum A, Lamche H, Küpeü Z, Jacobi A, Müller S, Hirt U, Adolf G R, Borges E. A novel Fc-engineered monoclonal antibody to CD37 with enhanced ADCC and high proapoptotic activity for treatment of B-cell malignancies. Blood. 2011 Oct. 13; 118(15):4159-68. Epub 2011 Jul. 27. Blood. 2011 Oct. 13; 118(15):4159-68. Epub 2011 Jul. 27.
Lazar G A, Dang W, Karki S, Vafa O, Peng J S, Hyun L, Chan C, Chung H S, Eivazi A, Yoder S C, Vielmetter J, Carmichael D F, Hayes R J, Dahiyat B I. Engineered antibody Fc variants with enhanced effector function. Proc Natl Acad Sci USA. 2006 Mar. 14; 103(11):4005-10. Epub 2006 Mar. 6.
Lu Y, Vernes J M, Chiang N, Ou Q, Ding J, Adams C, Hong K, Truong B T, Ng D, Shen A. Nakamura G, Gong Q, Presta L G, Beresini M, Kelley B, Lowman H, Wong W L, Meng Y G. Identification of IgG(1) variants with increased affinity to FcγRIIIa and unaltered affinity to FcγRI and FcRn: comparison of soluble receptor-based and cell-based binding assays. J Immunol Methods. 2011 Feb. 28; 365(1-2):132-41. Epub 2010 Dec. 23.
Mizushima T, Yagi H, Takemoto E, Shibata-Koyama M, Isoda Y, Iida S, Masuda K, Satoh M, Kato K, Structural basis for improved efficacy of therapeutic antibodies on defucosylation of their Fc glycans. Genes Cells. 2011 November; 16(11):1071-1080.
Moore G L, Chen H, Karki S, Lazar G A. Engineered Fc variant antibodies with enhanced ability to recruit complement and mediate effector functions. MAbs. 2010 March-April; 2(2):181-9.
Nordstrom J L, Gorlatov S, Zhang W, Yang Y, Huang L, Burke S, Li H, Ciccarone V, Zhang T, Stavenhagen J, Koenig S, Stewart S J, Moore P A, Johnson S, Bonvini E. Anti-tumor activity and toxicokinetics analysis of MGAH22, an anti-HER2 monoclonal antibody with enhanced Fc-gamma receptor binding properties. Breast Cancer Res. 2011 Nov. 30; 13(6):R123. [Epub ahead of print]
Richards J O, Karki S, Lazar G A, Chen H, Dang W, Desjarlais J R. Optimization of antibody binding to FcgammaRIIa enhances macrophage phagocytosis of tumor cells. Mol Cancer Ther. 2008 August; 7(8): 2517-27.
Schneider S, Zacharias M. Atomic resolution model of the antibody Fc interaction with the complement C1q component. Mol Immunol. 2012 May; 51(1):66-72.
Shields R L, Namenuk A K, Hong K, Meng Y G, Rae J, Briggs J, Xie D, Lai J, Stadlen A, Li B, Fox J A, Presta L G. High resolution mapping of the binding site on human IgG1 for Fc gamma R I, Fc gamma RII, Fc gamma RIII, and FcRn and design of IgG1 variants with improved binding to the Fc gamma R. J Biol Chem. 2001 Mar. 2; 276(9):6591-604.
Stavenhagen J B, Gorlatov S, Tuaillon N, Rankin C T, Li H, Burke S, Huang L, Vijh S, Johnson S, Bonvini E, Koenig S. Fc optimization of therapeutic antibodies enhances their ability to kill tumor cells in vitro and controls tumor expansion in vivo via low-affinity activating Fcgamma receptors. Cancer Res. 2007 Sep. 15; 67(18):8882-90.
Stewart R, Thom G, Levens M, Guler-Gane G, Holgate R, Rudd P M, Webster C, Jermutus L, Lund J. A variant human IgG1-Fc mediates improved ADCC. Protein Eng Des Sel. 2011 September; 24(9):671-8. Epub 2011 May 18.

### EXAMPLES

The examples below are given so as to illustrate the practice of this invention. They are not intended to limit or define the entire scope of this invention.

### Example 1: Preparation and Expression of Constructs

The following monovalent anti-Her2 antibodies and controls were prepared and tested:

1. OA1-Fab-Her2, a monovalent anti-Her2 antibody, where the Her2 binding domain is a Fab on chain A, and the Fc region is a heterodimer having the mutations T350V_L351Y_F405A_Y407V in Chain A, and T350V_T366L_K392L_T394W in Chain B; the epitope of the antigen binding domain is domain 4 of Her2.
2. OA2-Fab-Her2, a monovalent anti-Her2 antibody, where the Her2 binding domain is a Fab on chain B, and the Fc region is a heterodimer having the mutations T350V_L351Y_F405A_Y407V in Chain A, and T350V_T366L_K392L_T394W in Chain B; the epitope of the antigen binding domain is domain 4 of Her2.
3. OA3-scFv-Her2, a monovalent anti-Her2 antibody, where the Her2 binding domain is an scFv, and the Fc region is a heterodimer having the mutations L351Y_S400E_F405A_Y407V in Chain A, and T366I_N390R_K392M_T394W in Chain B; the epitope of the antigen binding domain is domain 4 of Her2.
4. FSA-scFv-Her2, a bivalent anti-Her2 antibody, where both Her2 binding domains are in the scFv format, and the Fc region is a heterodimer having the mutations L351Y_S400E_F405A_Y407V in Chain A, and T366I_N390R_K392M_T394W in Chain B; the epitope of the antigen binding domain is domain 4 of Her2.
5. FSA-Fab-Her2, a bivalent anti-Her2 antibody, where both Her2 binding domains are in the Fab format, and the Fc region is a heterodimer having the mutations T350V_L351Y_F405A_Y407V in Chain A, and T350V_T366L_K392L_T394W in Chain B; the epitope of the antigen binding domain is domain 4 of Her2.
6. wt FSA Hcptn, a wild-type Herceptin produced in-house in CHO as a control. The epitope of the antigen binding domain is domain 4 of Her2.
6A. Commercial Herceptin, a wild-type Herceptin purchased from Roche as a control. The epitope of the antigen binding domain is domain 4 of Her2.
7. OA4-scFv-BID2, a monovalent anti-Her2 antibody, where the Her2 binding domain is a scFv on chain A, and the Fc region is a heterodimer having the mutations L351Y_F405A_Y407V in Chain A, and T366L_K392M_T7394W in Chain B. The epitope of antigen binding domain is domain 1 of Her2.
8. FSA-scFv-BID2, a bivalent anti-Her2 antibody, where both Her2 binding domains are in the scFv format, and the Fc region is WT. The epitope of antigen binding domain is domain 1 of Her2.

With the exception of the Commercial Herceptin purchased from Roche, all antibodies expressed in CHO and described in Example 1 and Example 16, are fucosylated antibodies. The Commercial Herceptin antibody contains a greater percentage of afucosylation relative to the CHO produced antibodies.

These antibodies and controls were cloned and expressed as follows. The genes encoding the antibody heavy and light chains were constructed via gene synthesis using codons optimized for human/mammalian expression. The Fab sequences were generated from a known Her2/neu binding Ab (Carter P. et al. (1992) Humanization of an anti P185 Her2 antibody for human cancer therapy. Proc Natl Acad Sci 89, 4285.) and the Fc was an IgG1 isotype. The scFv sequences, FSA-scFv-Her2 and OA3-scFv-Her2 were generated from a known Her2/neu binding Ab (Findley et al. (1990) Characterization of murine monoclonal antibodies reactive to either the human epidermal growth factor receptor or HER2/neu gene product. Cancer Res., 50:1550). The scFv sequences, FSA-scFv-BID2 and OA4-scFv-BID2 were generated from a known Her2/neu binding Ab (Schier R. et al. (1995) In vitro and in vivo characterization of a human anti-c-erbB-2 single-chain Fv isolated from a filamentous phage antibody library. Immunotechnology 1,73).

The final gene products were sub-cloned into the mammalian expression vector pTT5 (NRC-BRI, Canada) and expressed in CHO cells (Durocher, Y., Perret, S. & Kamen, A. High-level and high-throughput recombinant protein production by transient transfection of suspension-growing CHO cells. Nucleic acids research 30, E9 (2002)).

The CHO cells were transfected in exponential growth phase (1.5 to 2 million cells/mL) with aqueous 1 mg/mL 25 kDa polyethylenimine (PEI, Polysciences) at a PEI:DNA ratio of 2.5:1. (Raymond C. et al. A simplified polyethylenimine-mediated transfection process for large-scale and high-throughput applications. Methods. 55(1):44-51 (2011)). In order to determine the optimal concentration range for forming heterodimers, the DNA was transfected in optimal DNA ratios of the heavy chain A (HC-A), light chain (LC), and heavy chain B that allow for heterodimer formation (e.g. HC-A/HC-B/LC ratios=25:25:50% (OAAs), 50:0:50% (WT hcptn), 25:25:50 (FSA-Fab-Her2), 50:50:0 (FSA-scFv-BID2) and 50:50:0 (OA4-scFv-BID2). Transfected cells were harvested after 5-6 days with the culture medium collected after centrifugation at 4000 rpm and clarified using a 0.45 μm filter.

### Example 2: Purification and Analysis of Antibodies

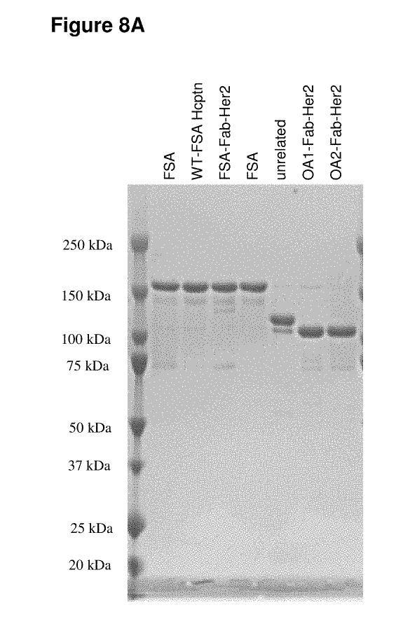

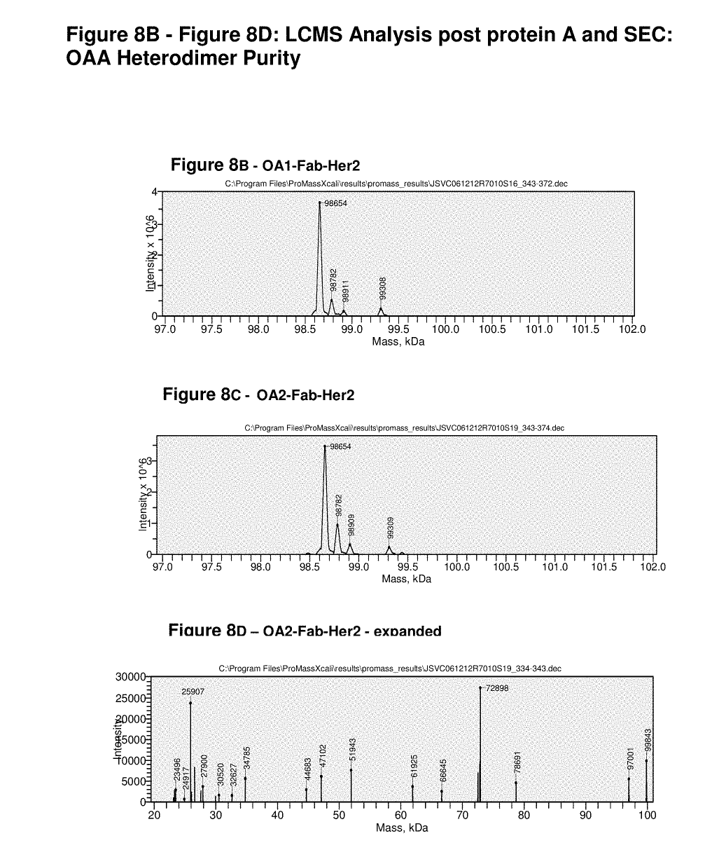

The monovalent anti-Her2 antibodies and control antibodies described above were purified as follows. The clarified culture medium was loaded onto a MabSelect SuRe (GE Healthcare) protein-A column and washed with 10 column volumes of PBS buffer at pH 7.2. The antibody was eluted with 10 column volumes of citrate buffer at pH 3.6 with the pooled fractions containing the antibody neutralized with TRIS at pH 11. FIG. 8A depicts the results of the SDS-PAGE analysis for wt FSA Hcptn, FSA-Fab-Her2, OA1-Fab-Her2, and OA2-Fab-Her2, after Protein-A purification. Lanes marked with “FSA” were loaded with a full size antibody (two Fab arms and an Fc region). The lane marked “unrelated” was loaded with an unrelated protein sample. Anti-Her2 OAAs express and purify to quantities and purities comparable to that of anti-Her2 FSA.

The protein-A antibody eluate was further purified by gel filtration (SEC). For gel filtration, 3.5 mg of the antibody mixture was concentrated to 1.5 mL and loaded onto a Sephadex 200 HiLoad 16/600 200 pg column (GE Healthcare) via an AKTA Express FPLC at a flow-rate of 1 mL/min. PBS buffer at pH 7.4 was used at a flow-rate of 1 mL/min. Fractions corresponding to the purified antibody were collected, concentrated to ˜1 mg/mL and stored at −80° C. The purified proteins were analyzed by LCMS as described in Example 8.

Antibodies purified by protein A chromatography and SEC were used for the assays described in the following Examples.

### Example 3: Monovalent Anti-HER2 Antibody (scFv) Shows Increased Concentration-Dependent Binding Density (Bₘₐₓ) Compared to Bivalent Anti-HER2 Antibody in SKOV3 Cells

The binding of an exemplary monovalent anti-Her2 antibody (OA3-scFv-Her2) was compared to that of a bivalent anti-Her2 antibody (FSA-scFv-Her2) in a Her2-expressing cell line, SKOV3, as described below. The SKOV cells line expresses the Her2 receptor at the 2+ level, and is considered to express the receptor with a medium density per cell. The monovalent antibodies tested in this example comprise an antibody-binding region that is an scFv.

Binding of the test antibodies to the surface of SKOV3 cells was determined by flow cytometry. Cells were washed with PBS and resuspended in DMEM at 1×10⁵ cells/100 μl. 100 μl cell suspension was added into each microcentrifuge tube, followed by 10 μl/tube of the antibody variants. The tubes were incubated for 2 hr 4° C. on a rotator. The microcentrifuge tubes were centrifuged for 2 min 2000 RPM at room temperature and the cell pellets washed with 500 μl media. Each cell pellet was resuspended 100 μl of fluorochrome-labelled secondary antibody diluted in media to 2 μg/sample. The samples were then incubated for 1 hr at 4° C. on a rotator. After incubation, the cells were centrifuged for 2 min at 2000 RPM and washed in media. The cells were resuspended in 500 μl media, filtered in tube containing 5 μl propidium iodide (PI) and analyzed on a BD LSRII flow cytometer according to the manufacturer's instructions.

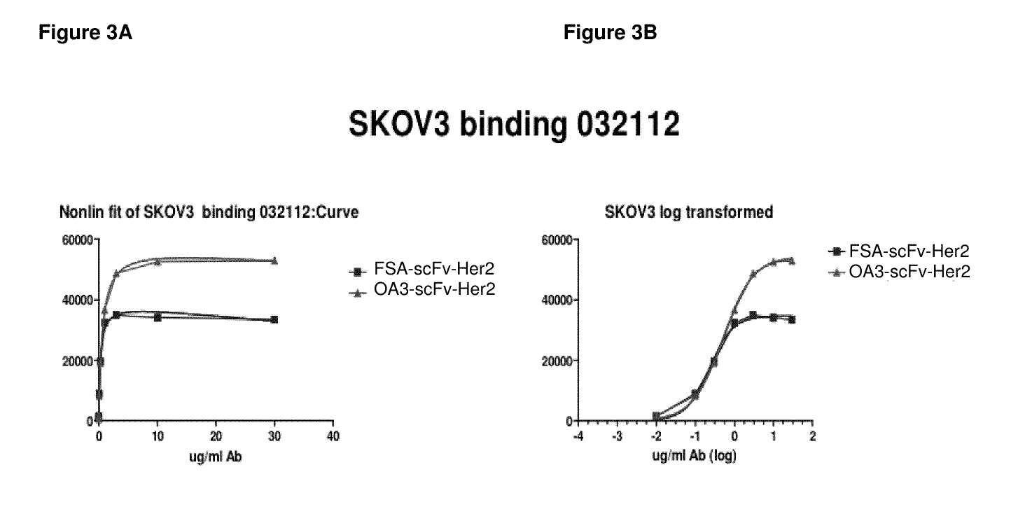

The results are depicted in FIGS. 3A and B and show that Anti-Her2 OA antibodies bind to SKOV3 cells in a concentration dependent manner with a higher binding density and Bₘₐₓ compared to anti-Her2 FSA (full-size antibody). Thus, more OA antibody molecules bind and decorate cells that display the Her2 antigen at the same concentration as the bivalent antibody. The OA Anti-Her2 antibodies tested in this example comprise scFv antigen binding domains, binding with a higher Bₘₐₓ compared to a FSA with bivalent scFv antigen binding domains.

### Example 4: Monovalent Anti-Her2 Antibody (Fab) Shows Higher Bₘₐₓ Compared to Bivalent Antibodies Independent of Her2 Density on Cells

The binding of exemplary monovalent anti-Her2 antibodies (OA1-Fab-Her2 and OA2-Fab-Her2) was compared to that of a bivalent anti-Her2 antibody (FSA-Fab-Her2), and wild type Herceptin™ (wt FSA Hcptn) in three Her2-expressing cell lines, MDA-MB-231, SKOV3, and SKBR3 as described below. The MDA-MB-231 cell line is considered to express Her2 with low density (0−1+), the SKOV3 cell line is considered to express Her 2 with medium density (2+), and the SKBR3 cell line is considered to express Her2 with high density (3+) (see Subik et al. (2010) Breast Cancer: Basic Clinical Research:4; 35-41, and Prang et a. (2005) British Journal of Cancer Research:92; 342-349). The monovalent antibodies tested in this example comprise an antibody-binding region that is a Fab.

Binding of the test antibodies to the surface of SKBR3 cells was determined by flow cytometry, as described in Example 2.

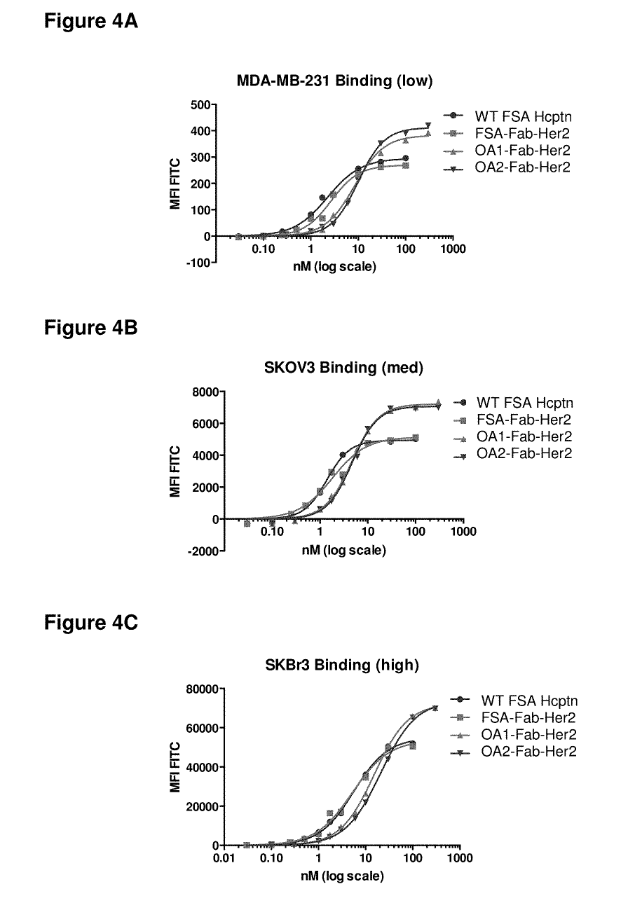

The results are depicted in FIGS. 4A-C, and values for K_{D} and Bₘₐₓ are shown in the tables below.

TABLE 1
Binding data in MDA-MB-231 cells

| Antibody | K_{D} (nM) | Bₘₐₓ |
|---|---|---|
| wt FSA Hcptn | 2.263 | 295 |
| FSA-Fab-Her2 | 2.717 | 269 |
| OA1-Fab-Her2 | 8.410 | 382 |
| OA2-Fab-Her2 | 9.973 | 412 |

TABLE 2
Binding data in SKOV3 cells

| | K_{D} | |
|---|---|---|
| Antibody | (nM) | Bₘₐₓ |
| wt FSA Hcptn | 1.407 | 4938 |
| FSA-Fab-Her2 | 1.826 | 5140 |
| OA1-Fab-Her2 | 4.667 | 7217 |
| OA2-Fab-Her2 | 4.725 | 7073 |

TABLE 3
Binding data in SKBR3 cells

| Antibody | K_{D} (nM) | Bₘₐₓ |
|---|---|---|
| wt FSA | 13.51 | 49814 |
| Hcptn | | |
| FSA-Fab- | 11.75 | 49421 |
| Her2 | | |
| OA1- | 10.93 | 64588 |
| Fab-Her2 | | |
| OA2- | 10.78 | 64835 |
| Fab-Her2 | | |

TABLE 4
Fold difference in binding - FSA-Fab-Her2 vs OA1-Fab-Her2

| Cell line | K_{D} | Bₘₐₓ |
|---|---|---|
| MDA-MB- | 3.09⇑ | 1.42⇑ |
| 231 | | |
| SKOV3 | 2.56⇑ | 1.40⇑ |
| SKBR3 | 2.91⇑ | 1.34⇑ |

Table 4 summarizes the fold difference in K_{D} and Bₘₐₓ between the FSA-Fab-Her2 vs OA1-Fab-Her2 for binding at saturation against cells lines with 1+, 2+ and 3+ Her2 receptor densities. The OA1-Fab-Her2 has a consistent approximately 1.4 fold increase in Bₘₐₓ vs. FSA-Fab-Her2 and a 3-fold increase in K_{D} across all cell lines tested.

FIG. 4 shows that the monovalent anti-Her2 antibodies have a higher binding density and Bₘₐₓ at concentrations where bivalent antibody binding is saturated; the increased OA binding density is independent of the density of Her2 on the cell. Anti-Her2 OAAs (one-armed antibodies) have a higher Bmax, compared to anti-Her2 FSA, on cells that display low (MDA-MB-231), medium (SKOV3) and high (SKBr3) Her2 density.

Anti-Her2 OAAs with Fab antigen binding domains, binding with a higher Bmax compared to a FSA with bivalent Fab antigen binding domains.

### Example 5: Monovalent Anti-HER2 Antibody Shows Increased ADCC Compared to Bivalent Anti-HER2 Antibody

The ability of an exemplary monovalent anti-Her2 antibody (OA1-Fab-Her2) to mediate ADCC compared to wt FSA Hcptn and FSA-Fab-Her2 was determined in SKBR3 cells as follows.

Overview: Target cells were pre-incubated with test antibodies (10 folds descending concentrations from 45 μg/mL) for 30 min followed by adding effector cells with effector/target cell ratio of 5:1 and the incubation continued for another 6 hours in 37° C./5% CO₂ incubators. Samples were tested with 8 concentrations, 10 folds descending from 45 ug/ml while the internal control Herceptin (wt FSA Hcptn) was titrated 10 fold descending from 10 μg/ml. LDH release was measured using LDH assay Kit.

Dose-response studies were performed with various concentrations of the samples with a pre-optimized Effector/Target (E/T) ratio (5:1). Half maximal effective concentration (EC₅₀) values were analyzed with the Sigmoidal dose-response non-linear regression fit by GraphPad Prism.

Cells were maintained in McCoy's 5A complete medium at 37° C./5% CO₂ and regularly sub-cultured with suitable medium supplemented with 10% FBS according to protocol from ATCC. Cells with passage number fewer than P10 were used in the assays. The samples were diluted to concentrations between 0.3-300 nM with Phenol red free MEM medium supplemented with 1% FBS and 1% Pen/strep prior to use in the assay.

ADCC Assay

SKBR3 target cells (ATCC, Cat# HTB-30) were harvested by centrifugation at 800 rpm for 3 minutes. The cells were washed once with assay medium and centrifuged; the medium above the pellet was completely removed. The cells were gently suspended with assay medium to make single cell solution. The number of SKBR3 cells was adjusted to 4× cell stock (10,000 cells in 50 μl assay medium). The test antibodies were then diluted to the desired concentrations as noted above.

The SKBR3 target cells were seeded in the assay plates as follows. 50 μl of 4× target cell stock and 50 μl of 4× sample diluents was added to wells of a 96-well assay plate and the plate was incubated at room temperature for 30 min in cell culture incubator. Effector cells (NK92/FcRγ3a(158V/V), 100 μl, E/T=5:1, i.e, 50,000 effector cells per well) were added to initiate the reaction and mixed gently by cross shaking. The plate was incubated at 37° C./5% CO2 incubator for 6 hours

Triton X-100 was added to cell controls without effector cells and antibody in a final concentration of 1% to lyze the target cells and these controls served as the maximum lysis controls. ADCC assay buffer (98% Phenol red free MEM medium, 1% Pen/Strep and 1% FBS) was added in to cell controls without effector cells and antibody and it served as the minimum LDH release control. Target cells incubated with effector cells without the presence of antibodies were set as background control of non-specific LDH release when both cells were incubated together. Cell viability was assayed with an LDH kit (Roche, cat#11644793001). The absorbance data was read at OD492 nm and OD650 nm on Flexstation 3.

Data Analysis

The percentages of cell lysis were calculated according the formula below:

Cell lysis %=100*(Experimental data−(E+T))/(Maximum release−Minimum release).

Data was presented and analyzed by Graphpad (v4.0).

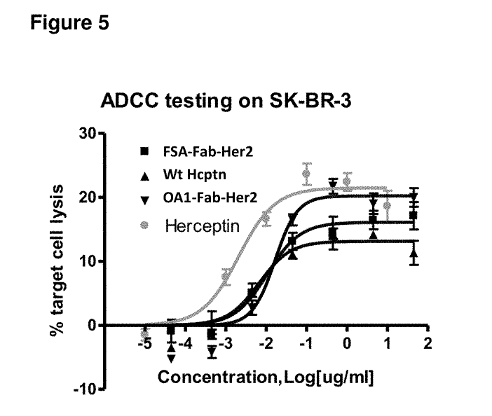

The dose-response curves are depicted in FIG. 5 and the EC₅₀ and maximum lysis for the antibodies tested is shown below in Table 5.

TABLE 5

| Antibody | EC₅₀ (ng/mL) | Max Lysis (%) |
|---|---|---|
| FSA-Fab-Her2 | 8.46 | 18.0 |
| wt FSA Hcptn | 2.83 | 17.4 |
| OA1-Fab-Her2 | 9.05 | 25.4 |

These results indicate that the monovalent asymmetric anti-Her2 antibody OA1-Fab-Her2 shows concentration-dependent lysis and higher maximum lysis compared to the bivalent antibody controls. Monovalent asymmetric anti-Her2 antibody OA1-Fab-Her2 shows higher % maximum of NK cell-mediated target cell lysis compared to the bivalent antibody controls (FSAs).

### Example 6: Monovalent Anti-HER2 Antibody Shows Increased CDC Compared to Bivalent Anti-HER2 Antibody

The ability of a monovalent anti-Her2 antibody to mediate CDC of SKBR3 cells compared to bivalent antibodies was determined as follows.

SKBR-3 cells were seeded at 2.5×106 vital cells in a T150 cell culture flask in 25 mL of DMEM/F-12 with 10% fetal calf serum. The cells were precultured by incubation at 37° C. and 5% CO2.

After five days of SKBR3 pre-culture, the cells were trypsinized and harvested. The cell suspension was rinsed over a separation filter to avoid cell clusters that could skew assay results. The cells were seeded in T25 suspension cell culture flasks at 1×10⁶ vital cells per mL. Anti-CIPS (complement-inhibiting-factors) antibodies (e.g. rat anti-CD59 and mouse anti-CD55) were added to the cell suspension at 10 μg antibody per 5×10⁶ vital cells. The cell suspension was incubated with anti-CIP-antibodies for 45 min and 5% CO₂.

Dilutions of test anti-Her2 antibodies were prepared and added to a white luminescence 96-well plate. The plate included wells containing controls for total cell lysis and controls for spontaneous lysis.

SKBR3 cells were harvested from the suspension flask and cell density and viability determined. A cell suspension was generated with a concentration of 4.0×10⁵ vital cells/mL. 50 μL of this suspension was seeded into the wells of the white luminescence 96-well plate as appropriate. The plate was incubated for 30 min at 37° C. and 5% CO₂. 10 μL of the serum was added into all wells and the plate incubated for 3:30 hours at 37° C. and 5% CO2.

Total cell lysis was induced as follows. Using the CytoTox-Glo Kit (Promega), 2 mL of assay buffer was mixed with 33.0 μL of Digitonin. 10 μL of this solution was added to each well of the total cell lysis controls. The plate was incubated for 30 min at 37° C. and 5% CO₂.

Read-out and analysis was performed as follows. Lyophilized substrate was reconstituted with 5 mL of assay buffer according to the CytoTox Glo Kit instructions (Promega). 50 μL of this solution was added to all 72 wells of the plate. The plate was incubated at room temperature for 15 min, and luminescence intensity determined using a TECAN Infinite F200 plate reader.

Specific cell lysis was calculated as follows:

Specific cell lysis[%]=[MFI(sample)−MFI(spontaneous)]/[MFI(total)−MFI(spontaneous)]×100.

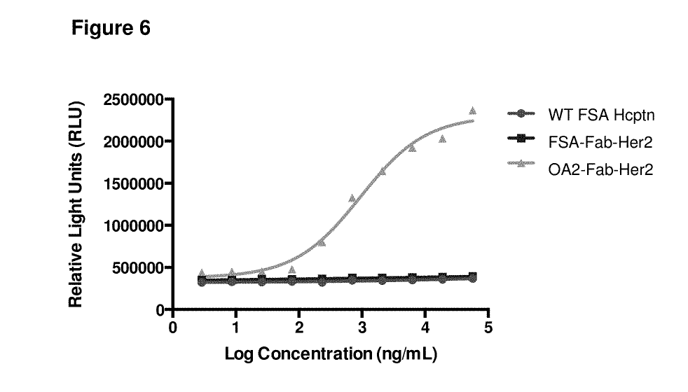

The results are shown in FIGS. 6A-C, and the EC₅₀, R2 and maximum lysis are shown in Table 6 below.

TABLE 6

| Antibody | EC50 ng/mL (n2-3) | R² (n2-3) | Max Lysis (%) (n2-3) |
|---|---|---|---|
| wt FSA Hcptn | 5516 | 0.7 | 12 |
| FSA-Fab-Her2 | 1740 | 0.9 | 11 |
| OA2-Fab-Her2 | 1247 | 0.9 | 343 |

These results indicate that the monovalent antibody tested shows increased concentration-dependent and higher CDC efficacy compared to bivalent antibodies at the same test concentrations. Anti-Her2 OAAs doses results in a higher complement dependent cytotoxicity against target cells, compared to anti-Her2 FSA.

### Example 7: Monovalent Anti-HER2 Antibody Shows Increased ADCP Compared to Bivalent Anti-HER2 Antibody

The ability of a monovalent anti-Her2 antibody to mediate ADCP of SKBR3 cells compared to bivalent antibodies was determined as follows.

ADCP Protocol

Overview: This protocol used in vitro differentiated macrophages that were co-cultured with PKH26-labeled target cells previously incubated with serial dilutions of antibodies. After 24 hr incubation, macrophages were stained with an APC (allophycocyanin)-conjugated anti-CD45 and/or CD11b antibody. Target cell phagocytosis was subsequently analyzed by flow cytometry.

The method was carried out as follows. PBMCs were prepared by density gradient centrifugation from leucapheresis material of healthy human donors. CD14 positive cells were separated using magnetic beads and seed at 2×10⁶ viable cells/mL in cell culture media. Macrophage differentiation was induced by the addition of 500 U/mL Granulocyte-macrophage colony-stimulating factor (GM-CSF). Cells were cultivated for 7 days total, and GM-CSF was added at day 3.

Marker expression of the cells was checked with anti-CD45, anti-CD11b, anti-CD14 and anti-CD16 antibodies by flow cytometric analysis.

Target cell line used was SKBR3. The presence of HER-2 was confirmed with Herceptin™ (Roche) and a FITC-conjugated anti-human IgG secondary antibody by flow cytometry. Target cells were stained with PKH26 (Sigma-Aldrich). The target cells were opsonized with serial 1:6 dilutions of test anti-Her2 antibodies (60 min) and incubated with macrophages in a ratio of 1:1 for 22 hrs.

Monocytes were stained with an APC-conjugated anti-CD45 and anti-CD11b antibody and analyzed by flow cytometry. Phagocytosis by CD45 positive cells was determined by PKH26 fluorescence intensity.

Controls per plate included (in duplicate): Target cell control of PKH26 stained SK-BR-3 cells only; Effector cell control of monocytes only; and Effector and target cells control with a non-specific IgG1 antibody. (Plate-specific background subtraction=effector and target cell control incubated with a non-specific isotype control antibody).

The percentage of antibody-dependent phagocytosis was determined by 1) setting the background reduced mean fluorescence intensity of the target cell control to % 100, and 2) setting the mean fluorescence intensity of the effector and target cell isotype control to 0%.

The following equation was used for calculating the percentage of antibody-dependent phagocytosis:

$$\% antibody \text{-} dependent phagocytosis = \frac{( B S M F {I}_{Sample} )}{( B S M F {I}_{target cell control} )} \times 100$$

$$B S M F I = background subtracted mean fluorescence intensity$$

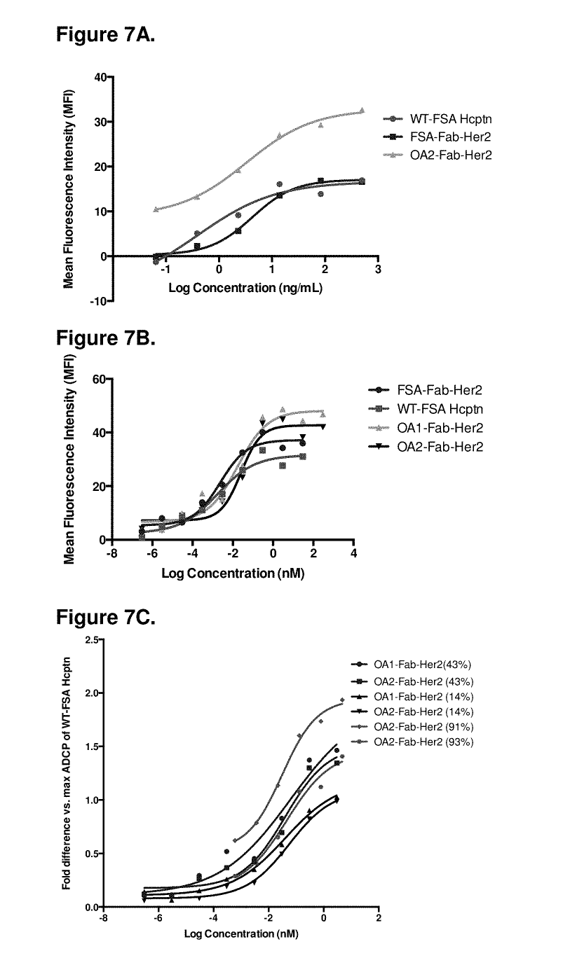

The results of this experiment are shown in FIGS. 7A to C, which show that the monovalent anti-Her2 antibody tested showed increased ADCP compared to bivalent anti-Her2 antibodies. FIG. 5 shows (A) Representative ADCP of donor 1 (91% CD16+ cells), (B) representative ADCP data from donor 1 study 2 (45% CD16+ cells), (C) All data plot (study 1 and 2 all donors) comparing fold difference of OA1-Fab-Her2 and OA2-Fab-Her2 over WT-FSA Hcptn based on percent CD16+ cells/donor. Anti-Her2 OAAs doses mediate a greater percent of antibody dependent cellular phagocytosis (of SKBr3 target cells) with in vitro differentiated macrophage as effector cells; ADCP efficacy is also a relation of effector:target cell ration with greater efficacy observed with higher numbers of effector macrophages FIG. 7C.

Table 7 provides data obtained from the plot in FIG. 7A.

TABLE 7
Average of Donor 1 and 2 (Donor 1, 91%; Donor 2, 93% CD16+)

| | | | Max Lysis |
|---|---|---|---|
| Variant | EC50 ng/mL | R² | (MFI) |
| wt FSA Hcptn | 1.2 | 0.95 | 18.0 |
| FSA-Fab-Her2 | 3.2 | 0.95 | 21.5 |
| OA2-Fab-Her2 | 3.0 | 0.97 | 35.4 |

Tables 8 and 9 provide data obtained from the plot in FIG. 7B

TABLE 8
Donor 1 (43% CD16+ enrichment)

| | | | Max Lysis |
|---|---|---|---|
| Variant | EC₅₀ (pM) | R2 | (MFI) |
| 506 | 2.35 | 0.96 | 37.2 |
| 792 | 1.72 | 0.94 | 31.6 |
| 1040 | 17.5 | 0.94 | 48.1 |
| 1041 | 25.3 | 0.94 | 42.7 |

TABLE 9
Donor 2 (14% CD16+ enrichment)

| | | | Max Lysis |
|---|---|---|---|
| Variant | EC₅₀ (pM) | R2 | (MFI) |
| 506 | 5.5 | 0.97 | 24.8 |
| 792 | 16.8 | 0.96 | 28.2 |
| 1040 | 36.7 | 0.99 | 34.9 |
| 1041 | 30.6 | 0.98 | 28.2 |

### Example 8: Purification and Yield of Monovalent Anti-Her2 Antibodies with a Heterodimeric Fc Region

The purification and yield of monovalent OA1-Fab-Her2 and OA2-Fab-Her2 were tested by LCMS after protein A and SEC purification as described in Example 2.

LCMS Analysis of Heterodimer Purity

The purity of exemplary monovalent anti-Her2 antibodies was determined using LCMS under standard conditions. The antibodies were deglycosylated with PNGasF prior to loading on the LC-MS. Liquid chromatography was carried out on an Agilent 1100 Series HPLC under the following conditions:

Flow rate: 1 mL/min split post column to 100 uL/min to MS

Solvents: A=0.1% formic acid in ddH₂O, B=65% acetonitrile, 25% THF, 9.9% ddH₂O, 0.1% formic acid

Column: 2.1×30 mm PorosR2

Column Temperature: 80° C.; solvent also pre-heated

Gradient: 20% B (0-3 min), 20-90% B (3-6 min), 90-20% B (6-7 min), 20% B (7-9 min)

Mass Spectrometry (MS) was subsequently carried out on an LTQ-Orbitrap XL mass spectrometer under the following conditions:

Ionization method: Ion Max Electrospray

Calibration and Tuning Method: 2 mg/mL solution of CsI is infused at a flowrate of 10 μL/min. The Orbitrap is then tuned on m/z 2211 using the Automatic Tune feature (overall CsI ion range observed: 1690 to 2800).

Cone Voltage: 40V

Tube Lens: 115V

FT Resolution: 7,500

Scan range m/z 400-4000

Scan Delay: 1.5 min

A molecular weight profile of the data was generated using Thermo's Promass deconvolution software.

The LC-MS results are shown in FIGS. 8B to D where FIG. 8B shows the LCMS analysis of OA1-Fab-Her2; FIG. 8C shown the LCMS analysis of OA2-Fab-Her2; and FIG. 8D is an expanded view of the LCMS spectrum of OA2-Fab-Her2 to show the detected contaminants at ˜0.8% Two Light chains+1 Short Heavy chain (72,898 Da), ˜0.7% Short Heavy chain alone (25,907 Da). With respect to FIG. 8B, the calculated MW of one-armed heterodimer is 98,653 Da (OA1-Fab-Her2 or OA2-Fab-Her2); the calculated MW of one-armed homodimer is 52,159 Da (one heavy chain only); and the calculated MW of full chain homodimer is 145,147 Da (two paired full sized heavy chains, A/A (in the case of OA1-Fab-Her2) or B/B (in the case of OA2-Fab-Her2).

With respect to FIG. 8C, the calculated MW of one-armed heterodimer is 98,653 Da; the calculated MW of one-armed homodimer is 51,815 Da; the calculated MW of full chain homodimer is 145,492 Da; the calculated MW of 1 short arm and 2 light chains is 72,898 Da; and the calculated MW of shorter heavy chain alone is 25,907 Da.

In summary, FIGS. 8 B-C demonstrate yield of purified monovalent anti-Her2 antibodies of >95% purity post protein A and size exclusion chromatography, as determined by LCMS analysis. The yield of OA1-Fab-Her2 was 100% of the heterodimer, post protein A and size exclusion chromatography, as determined by LCMS analysis. The yield of OA2-Fab-Her2 was >98.5% of the heterodimer, with 0.8% of a species with two light chains and 1 short heavy chain, and with 0.7% of a short heavy chain species alone.

TABLE 10
Summary of Purification data for OA1-Fab-Her2

| | Volume of | Titer | % capture | Yield/L | |
|---|---|---|---|---|---|
| | production | mg/liter | post | post | |
| Batch # | (ml) | HPLC | protein A | protein A | LCMS |
| 1 | 10000 | ND | ND | 22.3 | 100% one-armed |
| | | | | | heterodimer |
| 2 | 500 | 29 | 96.5 | 24.0 | 100% one-armed |
| | | | | | heterodimer |
| 3 | 500 | 49 | 97.9 | 48.8 | 100% one-armed |
| | | | | | heterodimer |
| 4 | 500 | ND | ND | 36 | 100% one-armed |
| | | | | | heterodimer |

### Example 9: Monovalent Anti-Her2 Antibodies are Internalized and Inhibit the Growth of Target Cells

The ability of monovalent anti-Her2 antibodies to be internalized by SKBR3 cells was tested as follows.

SKBR3 cells were plated at 2000-4000 cells/well in 96 well plates, 1000/well in DMEM. The plates were incubated at 37° C. O/N.

Cytotoxicity Studies/Growth Inhibition Assays

Test antibodies were diluted in media and added to the cells at 10 μl/well in triplicate. The plates were incubated for 3 days 37° C. Cell viability was measured using alamarBlue™ (BIOSOURCE # DAL1100). 100/of alamarBlue™ was added per well and the plates incubate at 37° C. for 2 hr. Absorbance was read at 530/580 nm.

Internalization Studies

Anti-human saporin conjugated secondary antibody (Fab-Zap human, Catalog #IT-51) was incubated with primary human antibody at equimolar concentrations prior to addition to cells according to manufacturer's protocol (Advanced Targeting Systems, San Diego, Calif.). Without removing the cell culture supernatant, 25 μl was added for 1 hr. The plates were washed with tap water 4 times and air dried at room temperature. 100 μl of 0.057% (wt/vol) SRB (Sulforhodamine B) was added to each well for 30 minutes. The plates were quickly rinsed 4 times with 1% (vol/vol) Acetic acid, and air dried at RT. 1000 of 10 mM Tris base solution (pH 10.5) was added, and the plates were shaken for 5 minutes. The OD was measured at 510 nm in a microplate reader.

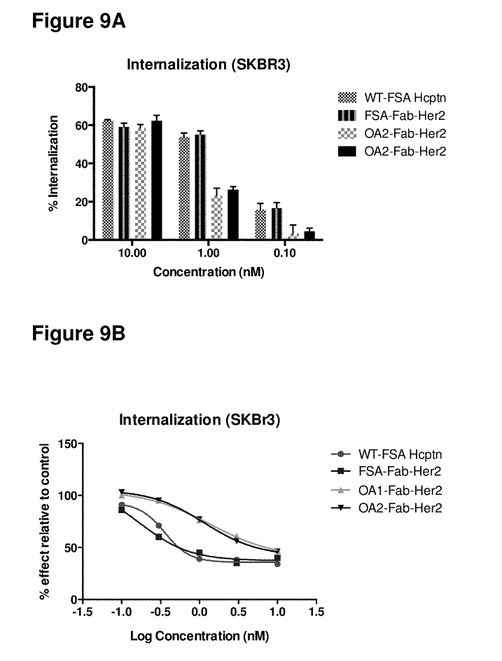

FIGS. 9A and B show the results of the internalization experiment. FIG. 9a shows the percent internalization of the antibodies tested, while FIG. 9b shows the data plotted as percent effect relative to control. This data indicates that the monovalent anti-Her2 antibodies tested are internalized by the target cell. Anti-Her2 OAAs and anti-Her2 FSAs have an equivalent % internalization of 60% at 10 nM.

Table 11 shows a summary of the data.

TABLE 11
Internalization data

| Antibody | % Max Effect | Max Effect (nM) |
|---|---|---|
| wt FSA Hcptn | 60 | 1 |
| FSA-Fab-Her2 | 60 | 1 |
| OA1-Fab-Her2 | 60 | 10 |
| OA2-Fab-Her2 | 60 | 10 |

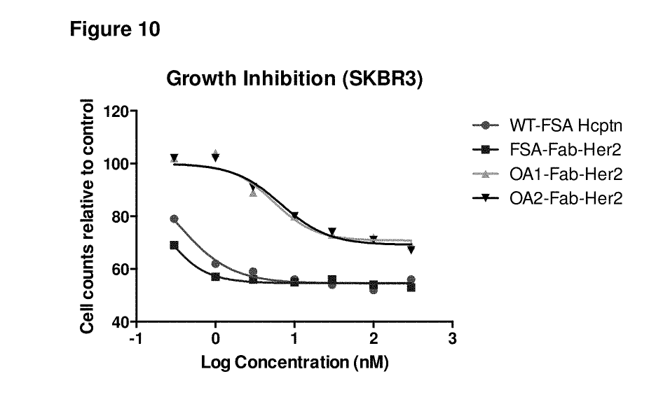

FIG. 10 shows the results of the cell growth assay. The monovalent anti-Her2 antibodies exhibit a maximum growth inhibition (of SKBR3 target cells) of 35% at 30 nM, compared to a max growth inhibition of 45% of anti-Her2 FSA at 1 nM. Table 12 provides a summary of the data.

TABLE 12

| | % Max Effect | Max Effect nM |
|---|---|---|
| Antibody | (n = 2) | (n = 2) |
| wt FSA Hcptn | 45 | 1 |
| FSA-Fab-Her2 | 45 | 1 |
| OA1-Fab-Her2 | 35 | 30 |
| OA2-Fab-Her2 | 35 | 30 |

### (a) Example 10: Monovalent Anti-Her2 Antibodies Bind to FcRn with an Equivalent K_{D}

The ability of monovalent anti-Her2 antibodies to bind to FcRn was tested by SPR as follows.

FcRn was immobilized via standard NHS/EDC coupling onto a BioRad GLM chip to about 3000 RUs. The antibody variants were injected at a flow rate of 50 ul/min for 120 seconds with a 300 second dissociation. A concentration series of 100 nM, 33.3 nM, 11.1 nM 3.7 nM, 1.23 nM and a buffer blank for double referencing. Sensorgrams were analysed using an equilibrium fit model in Proteon Manager.

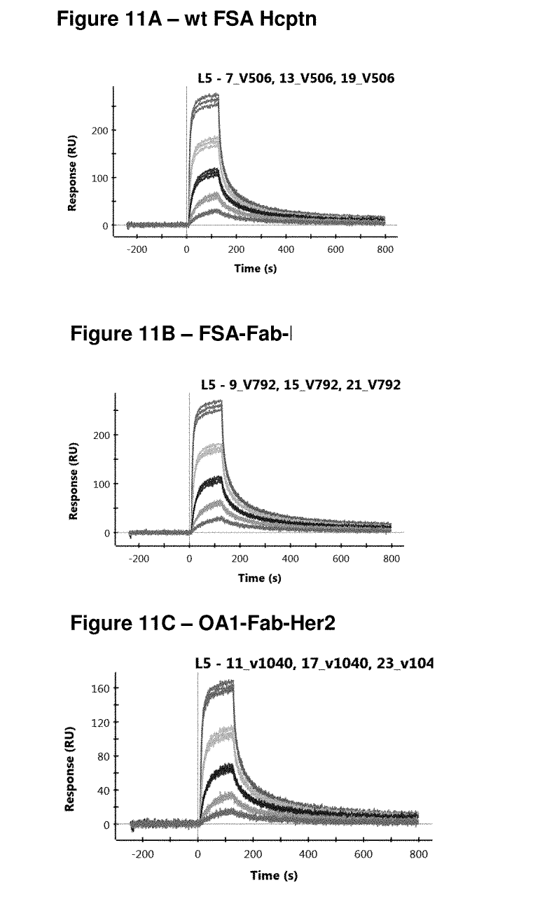

The results are shown in FIGS. 11A (wt FSA Hcptn), 9B (FSA-Fab-Her2), and 11C (OA1-Fab-Her2). These Figures indicate that the monovalent anti-Her2 antibody and bivalent anti-Her2 antibodies bind to the FcRn with an equivalent K_{D}. A summary of the results is found in Table 13 below.

TABLE 13

| | APPARENT | |
|---|---|---|
| | KD AVG | Std |
| Sample | (M) | Deviation |
| Wt FSA | 1.97E−08 | 8.E−10 |
| Hcptn | | |
| FSA-Fab- | 2.03E−08 | 6.E−10 |
| Her2 | | |
| OA1-Fab- | 2.21E−08 | 3.E−10 |
| Her2 | | |

### Example 11: Monovalent Anti-HER2 Antibody (scFv) Shows Increased Concentration-Dependent Binding Density (Bₘₐₓ) Compared to Bivalent Anti-HER2 Antibody in SKOV3 Cells

The binding of another exemplary monovalent anti-Her2 antibody (OA4-scFv-BID2) was compared to that of the corresponding bivalent anti-Her2 antibody (FSA-scFv-BID2), and other monovalent anti-Her2 antibodies in a Her2-expressing cell line, SKOV3, as described below. As indicated elsewhere, the SKOV cell line expresses the Her2 receptor at the 2+ level, which is considered to be medium density per cell. Binding assays were carried out as described in Example 3.

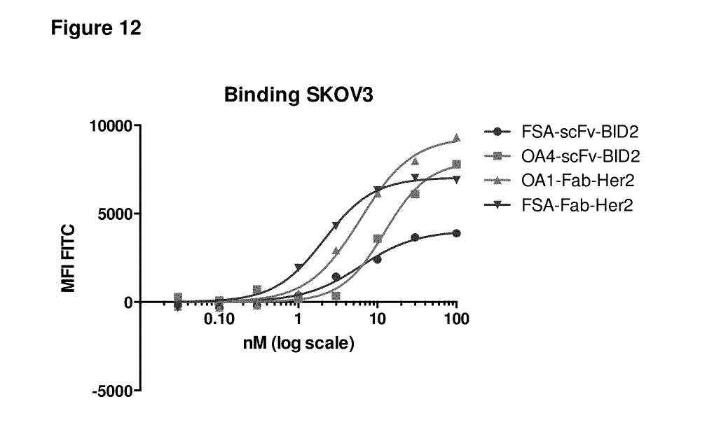

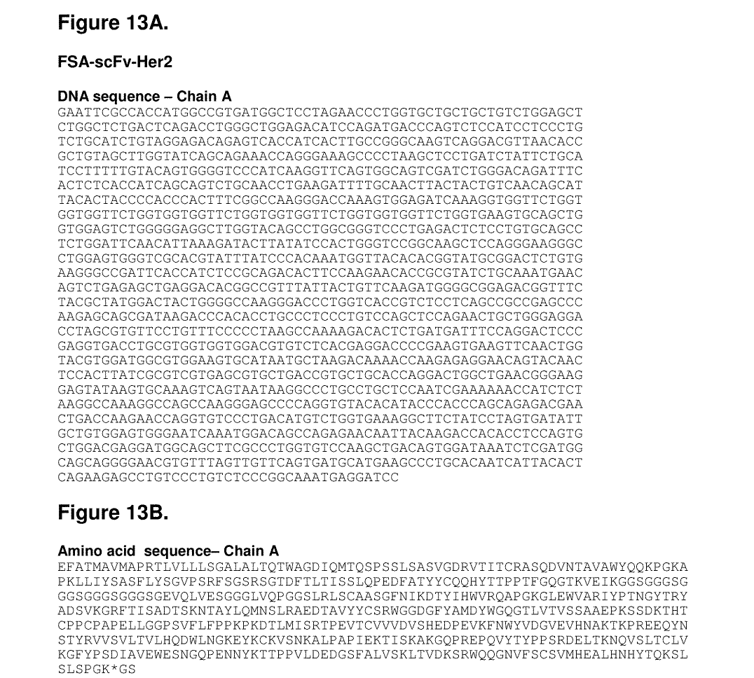

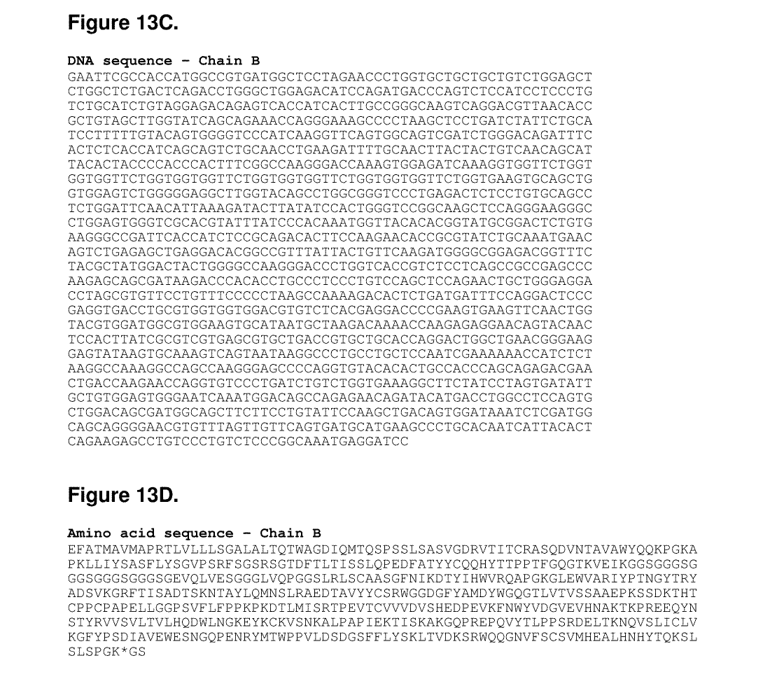

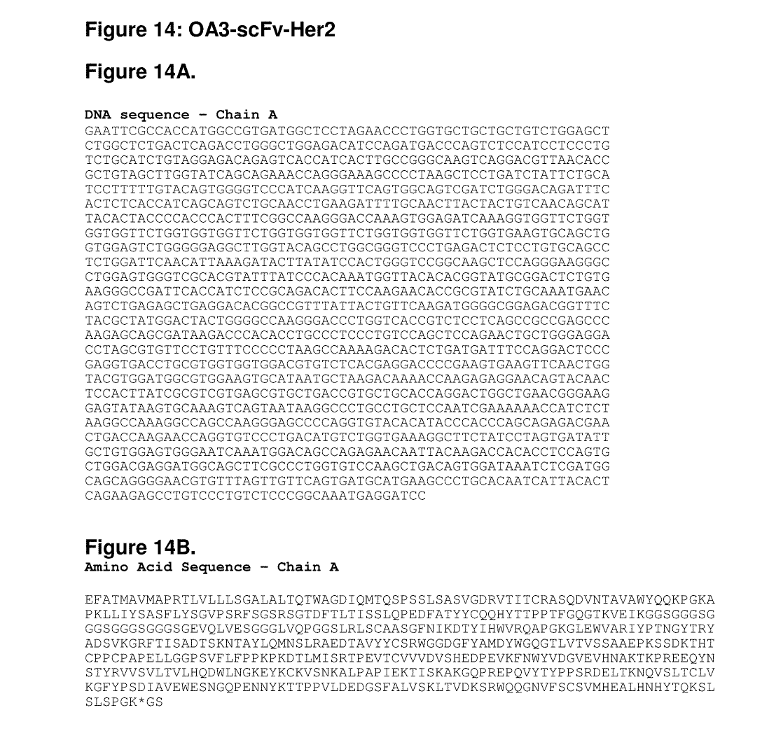

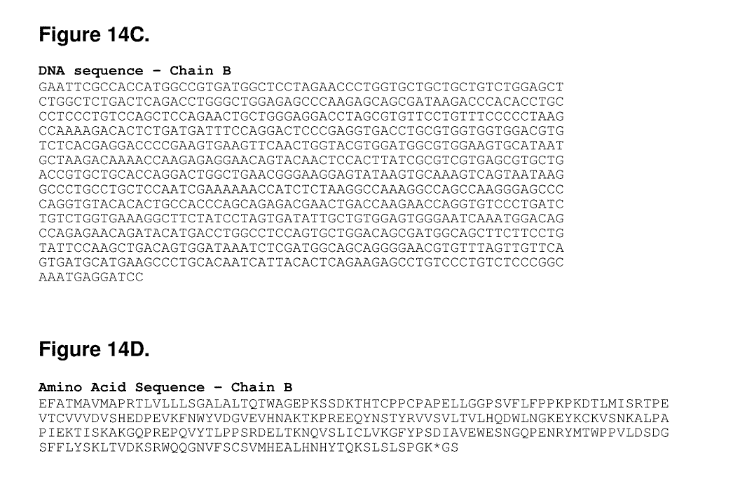

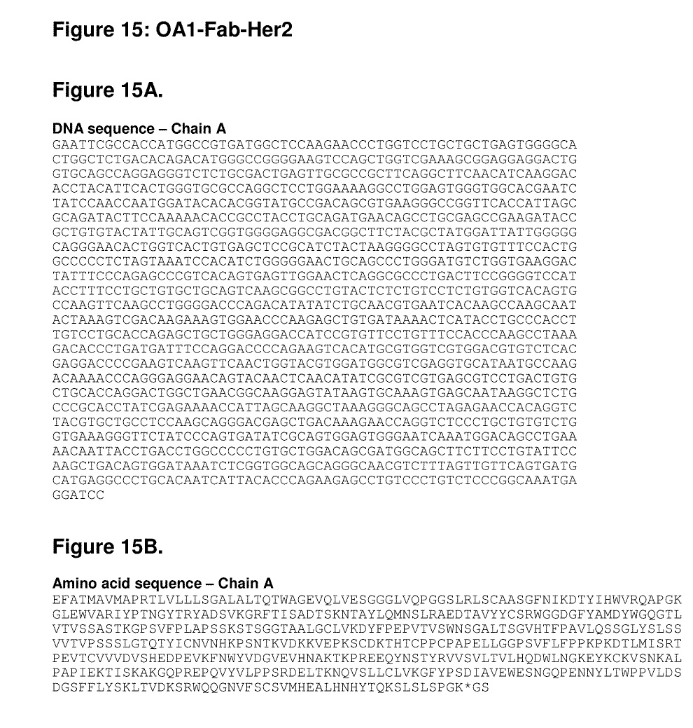

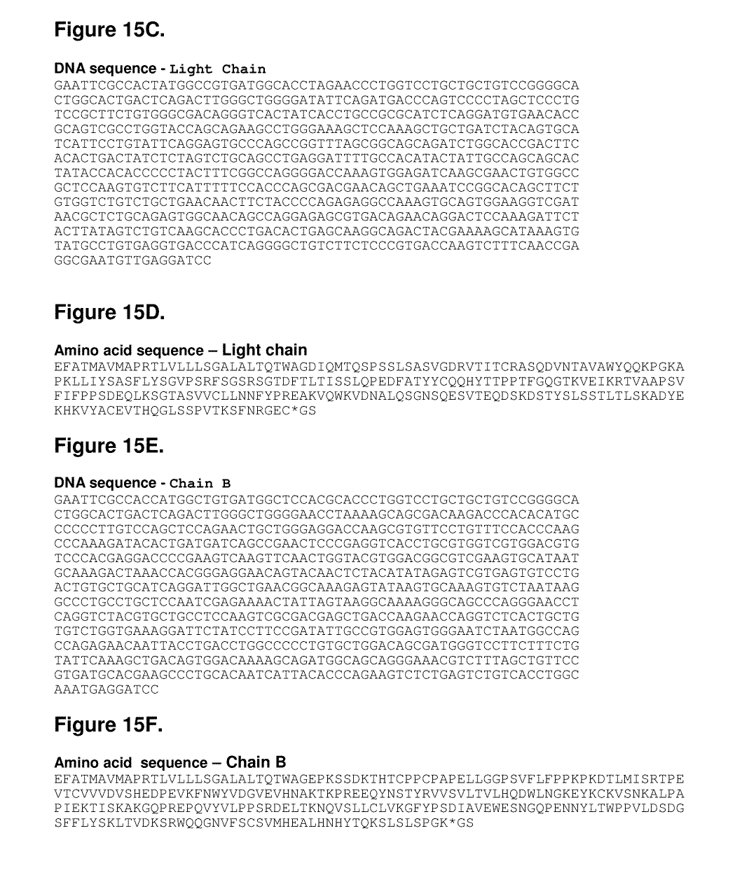

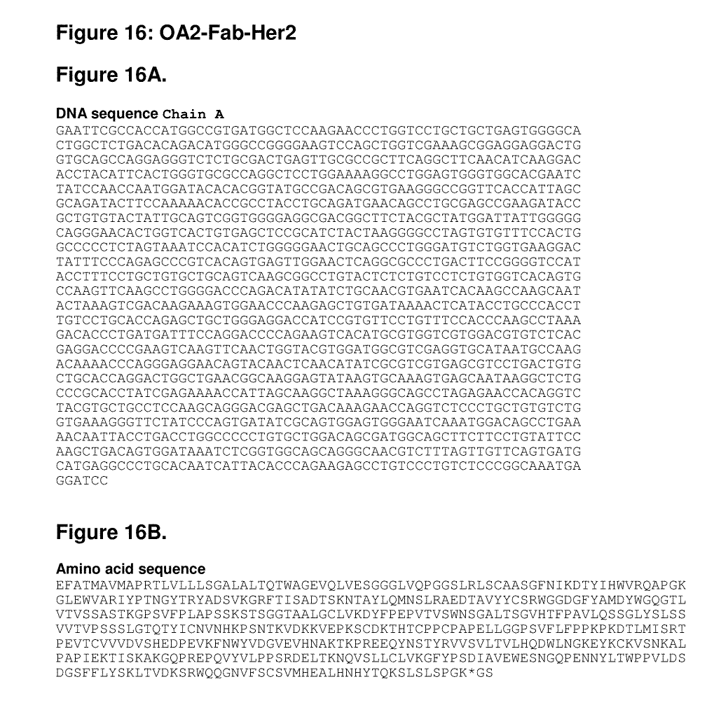

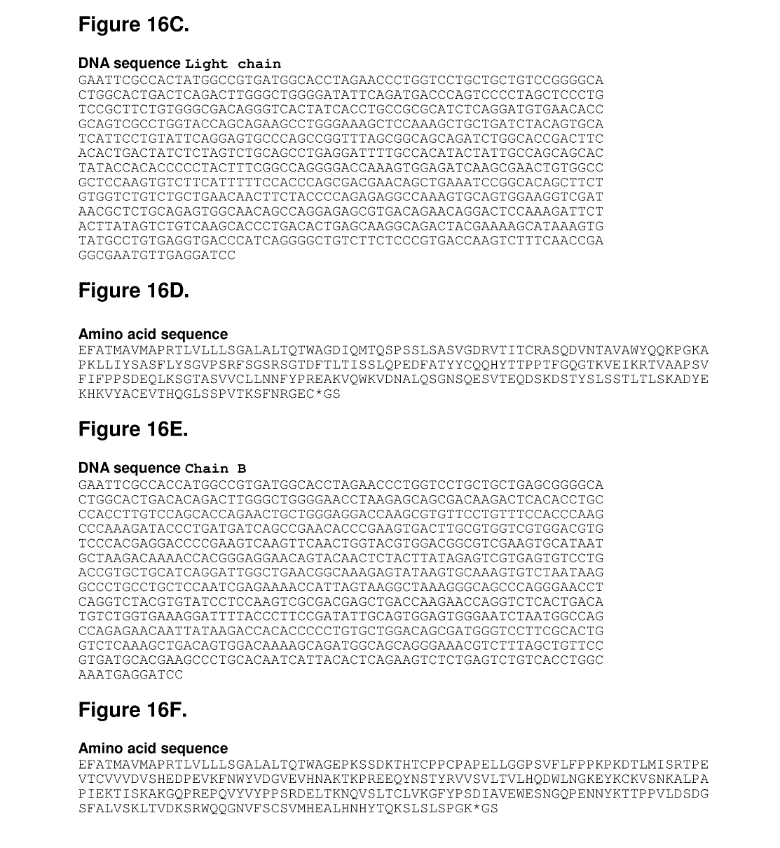

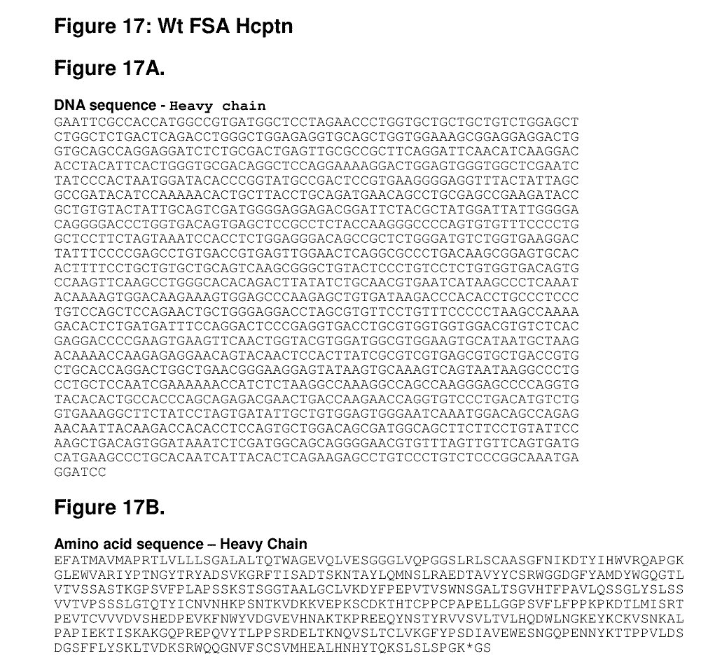

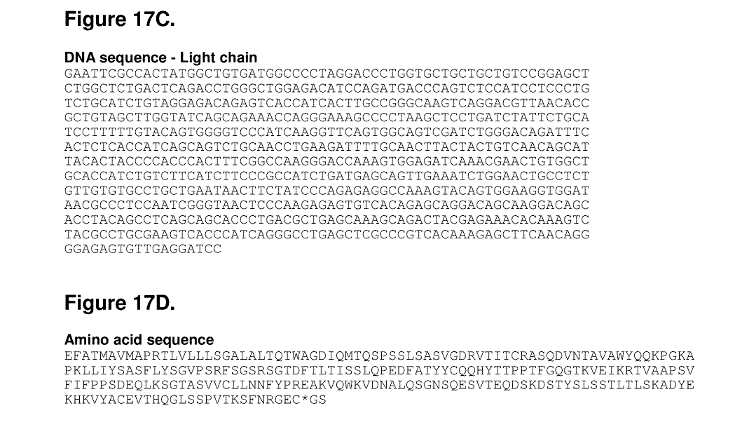

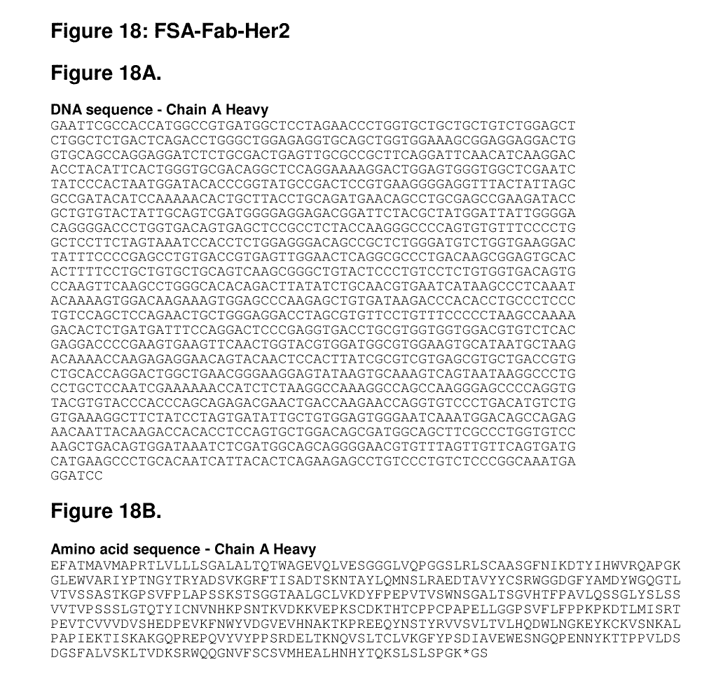

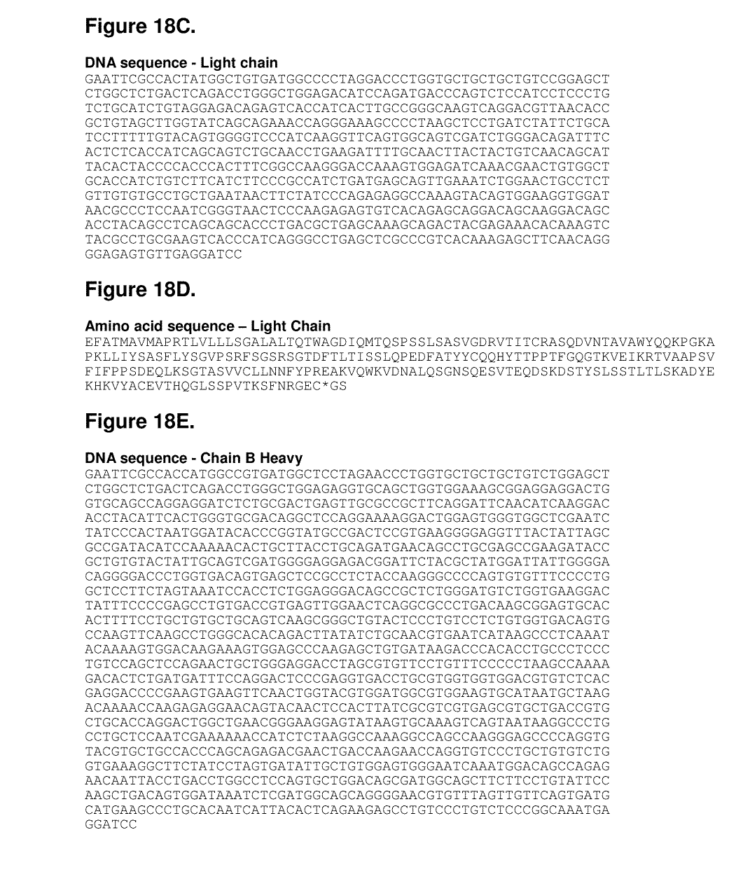

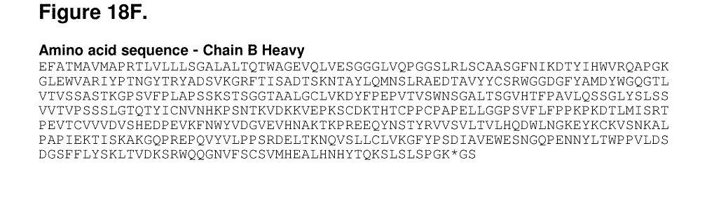

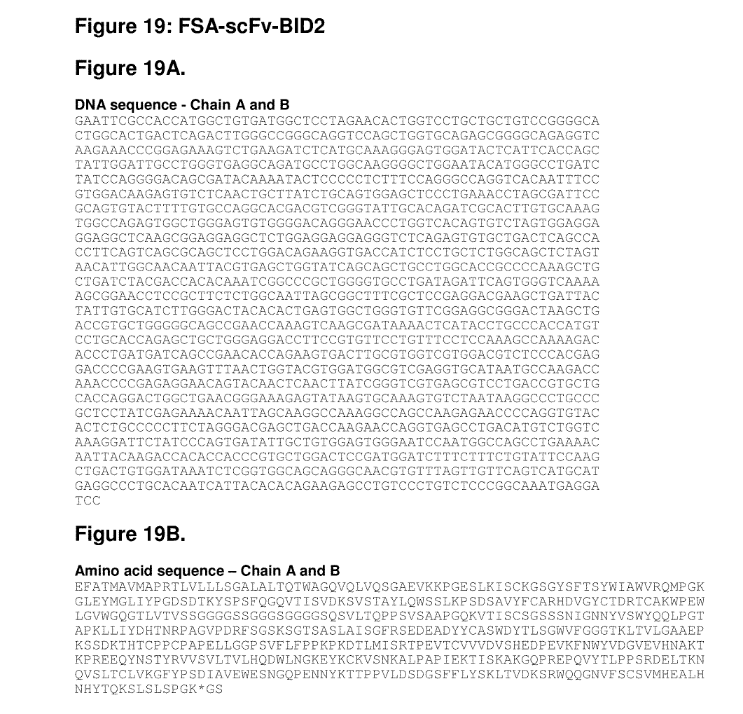

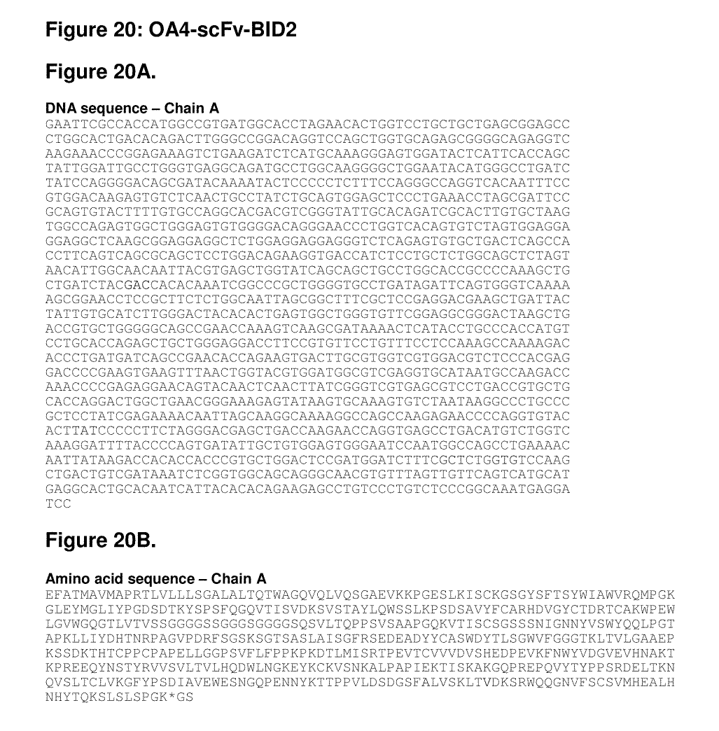

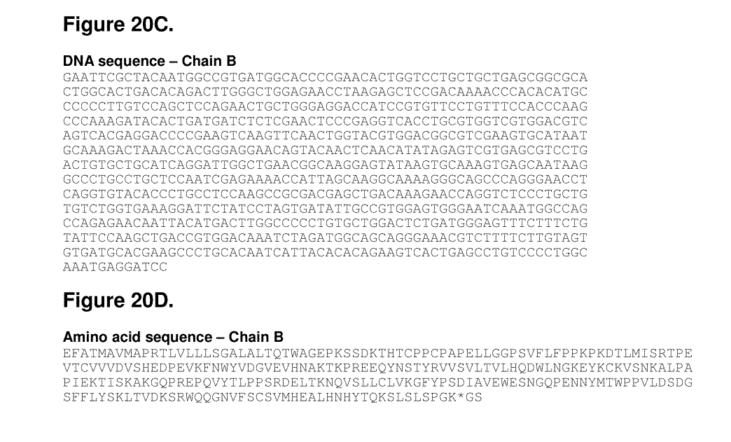

The results are shown in FIG. 12 and summarized in Tables 14 and 15. The results demonstrate that the monovalent anti-Her2 antibody OA4-scFv-BID2 has a higher Bₘₐₓ vs. compared to the bivalent FSA-scFv-BID2, and that ° A1-Fab-Her2 has higher Bmax vs. OA4-scFv-BID2 at equimolar concentrations.

TABLE 14
Summary of binding characteristics for tested antibodies

| Antibody | K_{D} (nM) | Bₘₐₓ |
|---|---|---|
| 792 | 2.117 | 7038 |
| 1040 | 6.005 | 9321 |
| 876 | 6.123 | 4048 |
| 878 | 12.45 | 7946 |

TABLE 15
Fold difference in binding for tested antibodies

| Comparison | K_{D} | Bₘₐₓ |
|---|---|---|
| FSA-Fab-Her2 vs. OA1-Fab-Her2 | 2.83⇑ | 1.32⇑ |
| FSA-scFv-BID2 vs. OA4-scFv-BID2 | 2.03⇑ | 1.96⇑ |

### Example 12: Monovalent Anti-Her2 Antibody Shows Increased ADCC in Triple Negative and Her2 1+ Cell Lines

The ability of an exemplary monovalent anti-Her2 antibody (OA1-Fab-Her2) to mediate ADCC compared to wt FSA Hcptn and FSA-Fab-Her2 was determined in the triple negative cell line MDA-MD-231 and in the Her2 1+ cell line MCF7 according to the protocol described in Example 5. MDA-MD-231 cells were grown in DMEM media, while the MCF7 cells were grown in Eagle's Minimum Essential Medium (Gibco #11095); both were supplemented with 0.01 mg/ml human recombinant insulin (Invitrogen), 10% FBS (Gibco#10099) and 1% non-essential amino acids (Gibco#11140).

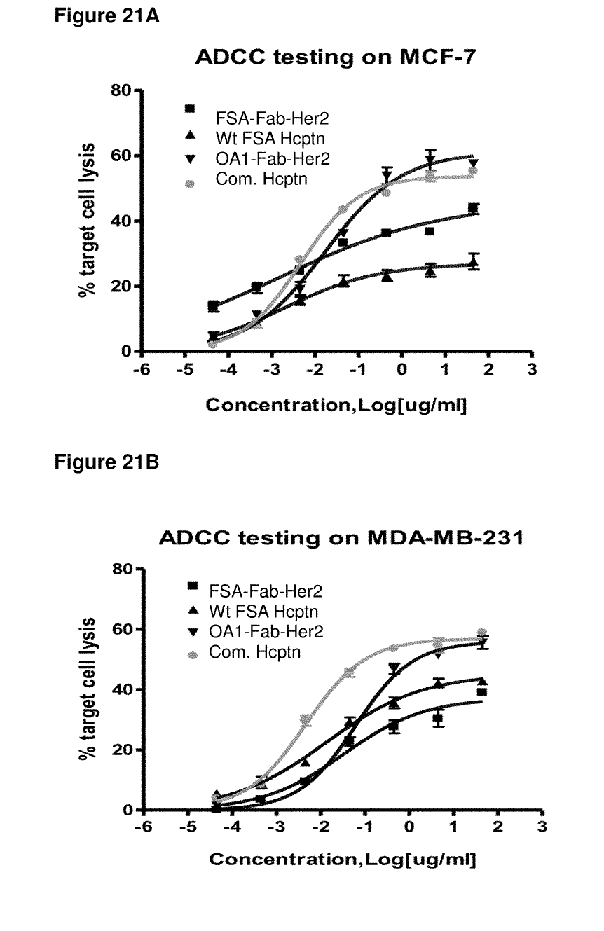

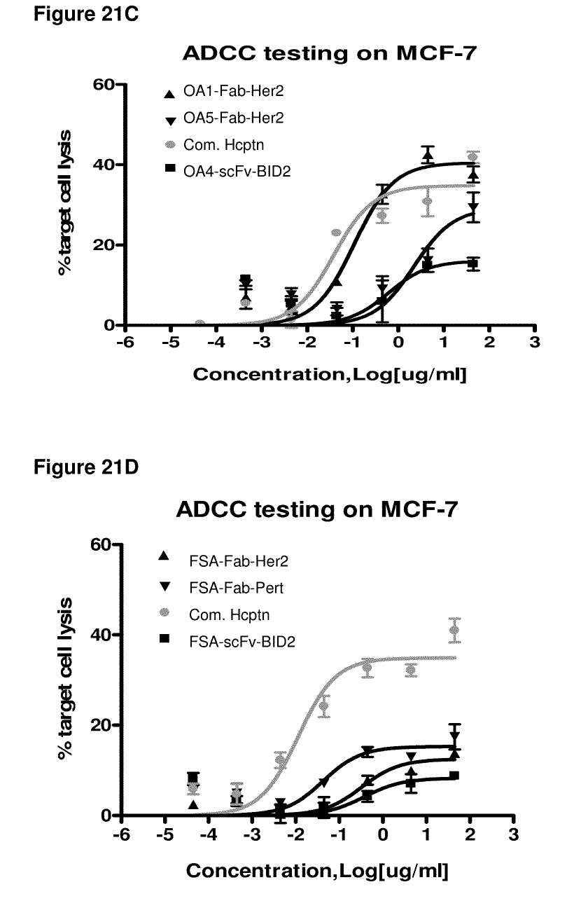

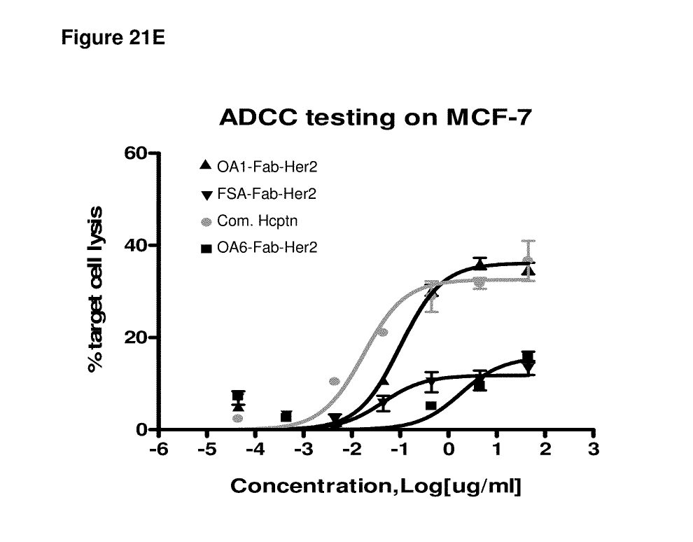

The dose-response curves are depicted in FIG. 21A (MCF7 cells) and FIG. 21B (MDA-MD-231) and the EC₅₀ and maximum lysis for the antibodies tested is shown in Tables 16 and 17.

TABLE 16
EC₅₀ and maximum lysis (MCF7 cells)

| | EC₅₀ | Max Lysis |
|---|---|---|
| Antibody | (ng/mL) | (%) |
| Wt FSA Hcptn | 2.05 | 26.9 |
| FSA-Fab-Her2 | 1.65 | 45.8 |
| OA1-Fab-Her2 | 17.0 | 61.1 |

These results indicate that the fold difference in EC₅₀ for FSA-Fab-Her2 vs. OA1-Fab-Her2 was 10.3 (increase), while the fold increase in Maximum lysis was 1.3 (increase).

TABLE 17
EC₅₀ and maximum lysis (MDA-MD-231 cells)

| | EC₅₀ | Max Lysis |
|---|---|---|
| Variant | (ng/mL) | (%) |
| Wt FSA Hcptn | 13.7 | 45.1 |
| FSA-Fab-Her2 | 33.4 | 37.2 |
| OA1-Fab-Her2 | 61.0 | 55.9 |

The fold difference in EC₅₀ for FSA-Fab-Her2 vs. OA1-Fab-Her2 was 1.8 (increase), while the fold increase in Maximum lysis was 1.5 (increase) in MDA-MD-231 cells.

### Example 13: Monovalent Anti-Her2 Antibody has a Broader Distribution (Vss) and t1/2 β Compared to FSA

The pharmacokinetics (PK) of an exemplary monovalent anti-Her2 antibody (OA1-Fab-Her2) were examined and compared to that of the control bivalent anti-Her2 antibody (wt FSA Hcptn). These studies were carried out as described below

Strain/gender: CD-1 Nude/male

Target body weight of animals at treatment: 0.025 kg

Number of animals: 12

Body weight: Recorded on the day prior to treatment for calculation of the volume to be administered.

Clinical signs observation: Up to 2 h post-injection and then twice daily from Day 1 to Day 11.

Mice were administered on Day 1 by an IV injection into the tail vein with the test article at a dose of 10 mg/kg. Blood samples, approximately 0.060 mL, were collected from the submandibular or saphenous vein at selected time points (3 animals per time points) up to 240 h post-dose as per the tables below. Pre-treatment serum samples (Pre-Rx) were obtained from a naïve animal. Blood samples were allowed to clot at room temperature for 15 to 30 minutes. Blood samples were centrifuged to obtain serum at 2700 rpm for 10 min at room temperature and the serum stored at −80° C. For the terminal bleed, blood was collected by cardiac puncture.

Dose level: 10 mg/kg

| | Time point | | | | | | | | | | | |
|---|---|---|---|---|---|---|---|---|---|---|---|---|
| | 15 | 30 | 1 | 6 | 12 | 24 | 36 | 48 | 72 | 96 | 168 | 240 |
| | min | min | h | h | h | h | h | h | h | h | h | h |
| Ani- | √ | | √ | | √ | | √ | | √ | | √T | |
| mal | | | | | | | | | | | | |
| No. | | | | | | | | | | | | |
| 1, | | | | | | | | | | | | |
| 2, 3 | | | | | | | | | | | | |
| Ani- | | √ | | √ | | √ | | √ | | √ | | √T |
| mal | | | | | | | | | | | | |
| No. | | | | | | | | | | | | |
| 4, | | | | | | | | | | | | |
| 5, 6 | | | | | | | | | | | | |

√T: Terminal bleed by cardiac puncture.

Serum concentrations were determined by ELISA. Briefly, Her2 was coated at 0.5 ug/ml in PBS, 25 ul/well in a HighBind 384 plate (Corning 3700) plate and incubated overnight at 4° C. Well were washed 3× with PBS-0.05% tween-20 and blocked with PBS containing 1% BSA, 80 ul/well for 1-2 h at RT. Dilution of antibody serum and standards were prepared PBS containing 1% BSA. Following blocking, the block was removed and the antibody dilutions were transferred to the wells. The ELISA plate was centrifuged 30 sec at 1000 g to remove bubbles and the plate was incubated at RT for 2 h. The plate was washed 3× with PBS-0.05% tween-20 and 25 ul/well of AP-conjugated goat anti-human IgG, Fc (Jackson ImmunoResearch)_was added (at a 1:5000 dilution in PBS containing 1% BSA) and incubated 1 h at RT. The plate was washed 4× with PBS-0.05% tween-20 and 25 ul/well of AP substrate (1 tablet in 5.5 mL pNPP buffer) was added. Using the Perkin Elmer Envision reader, read OD at 405 nm at different time intervals (0-30 minutes). The reaction was stopped with addition of 5 uL of 3N NaOH before OD405 reach 2.2. The plate was centrifuged for 2 minutes at 1000 g before performing the last reading.

Serum concentrations were analysed using the WinnonLin software version 5.3 to obtain PK parameters. Serum samples were analyzed in two set of multiple dilutions and results within the validated range were accepted and averaged. Serum concentration values below the Lower Limit of Quantification (LLOQ) following ELISA analysis, were considered as 0 for the calculation of the mean serum concentration. The LLOQ obtained from the ELISA assays was approximately 1.2 μg/mL.

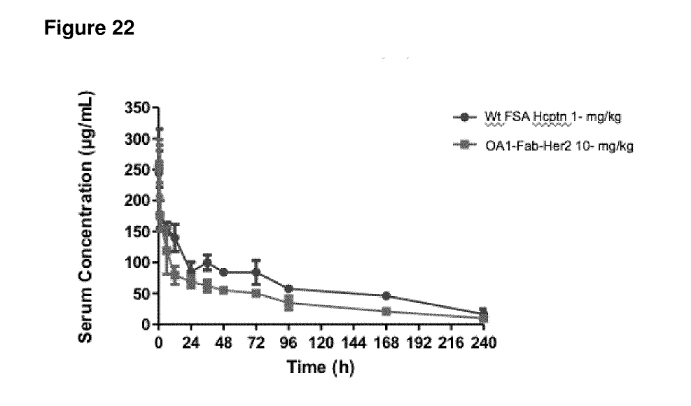

The results are shown in FIG. 22 and the PK parameters tested are shown in Table 18.

TABLE 18
PK Parameters

| | WT | | OA1- | |
|---|---|---|---|---|
| | FSA | | Fab- | |
| | Hcptn | | Her2 | |
| Parameters | 10 mg/kg | % CV | 10 mg/kg | % CV |
| α (1/h) | 1.104 | 49.89 | 0.8065 | 32.93 |
| β (1/h) | 0.0089 | 23.29 | 0.0115 | 26.72 |
| k₁₀ (1/h) | 0.0181 | 22.38 | 0.0329 | 21.75 |
| k₁₂ (1/h) | 0.5515 | 59.20 | 0.5031 | 36.46 |
| k₂₁ (1/h) | 0.5437 | 44.13 | 0.2820 | 32.37 |
| C₀ (μg/mL) | 292.5 | 12.57 | 301.4 | 8.52 |
| AUC | 16134 | 17.93 | 9158 | 19.49 |
| (μg · h/mL) | | | | |
| MRT (h) | 111.1 | 23.28 | 84.60 | 26.88 |
| V_{c} (mL/kg) | 34.19 | 12.58 | 33.17 | 8.53 |
| Vₚ (mL/kg) | 34.69 | 20.91 | 59.20 | 18.07 |
| CL | 0.620 | 17.95 | 1.092 | 19.51 |
| (mL/h/kg) | | | | |
| Vₛₛ (mL/kg) | 68.88 | 8.96 | 92.37 | 11.38 |
| t_{1/2} α (h) | 0.628 | 49.85 | 0.8594 | 32.91 |
| t_{1/2} β (h) | 77.68 | 23.27 | 60.24 | 26.71 |

The results shown in FIG. 22 indicate that the monovalent anti-Her2 antibody tested has reasonable PK parameters for dosing in humans. Notably, the monovalent anti-Her2 antibody has a greater Vss (volume at steady state), indicating that the antibody is distributed in a greater volume and has a greater distribution into the tissues.

### Example 14: Monovalent Anti-Her2 Antibody Treatment Reduces Phosphorylation of Erb2 and MAPK in SKBr3 Cells

The effect of treatment of SKBr3 with an exemplary monovalent anti-Her2 antibody (OA1-Fab-Her2) on phosphorylation of signaling molecules was determined as described below.

For the detection of phosphorylation by western immunoblotting, 12-well plates were seeded with 50,000 cells/well in serum-containing media and incubated at 37° C. After 24 h the media was replaced and antibody treatments were added to wells at final concentration of 100 nM and the plate incubated for 30 min at 37° C. Following the antibody incubation, appropriate wells were treated with rhHRGβ1 in media at 1 nM for 15 min. The treatment was stopped by placing the plates on ice, aspirating the media and washing the wells with ice-cold dPBS. Lysis-M buffer was added (500 well) and incubated at RT for 5 min with gentle shaking.

Cell lysate was centrifuged at 14,000 g for 10 min and the cell lysate was removed and stored in reducing or non-reducing buffer and boiled for 5 min (reducing sample). BCA protein determination was completed with remaining crude cell lysate following the manufacturer's instruction. An SDS-PAGE gel was loaded with 3 μg/well and transferred onto a Immobilon-P PVDF membrane. The membrane was washed in zenopure water, immersed in methanol for 2 minutes and air dried overnight (or 1 hour RT). The membrane was incubated with the appropriate primary antibodies (mouse anti-PY20 ZYMED, Invitrogen; Rabbit anti-ErbB2; Rabbit anti-total Akt; Rabbit anti-P-Akt (Ser473); Rabbit anti-p44/p42; Rabbit anti-P-p44/p42, Cell Signaling Technologies) at 4° C. overnight. Membranes were washed 4×20 min in TBS-T and incubated with the secondary antibodies (HRP-conjugated goat anti-mouse IgG; HRP-conjugated donkey anti-rabbit IgG; Jackson ImmunoResearch) for 30 min at RT with gentle orbital shaking. Membranes were washed 4×20 min in TBS-T and rinsed with water before the addition of ECL substrate. Films are exposed at various times and developed with AFP mini-med 90.

For the detection of p-AKT, the PathScan Phospo-AKT Sandwich ELISA kit (Cell Signaling Technology, cat. no 7252) was used and protocol followed as detailed in the manufacturer's instructions.

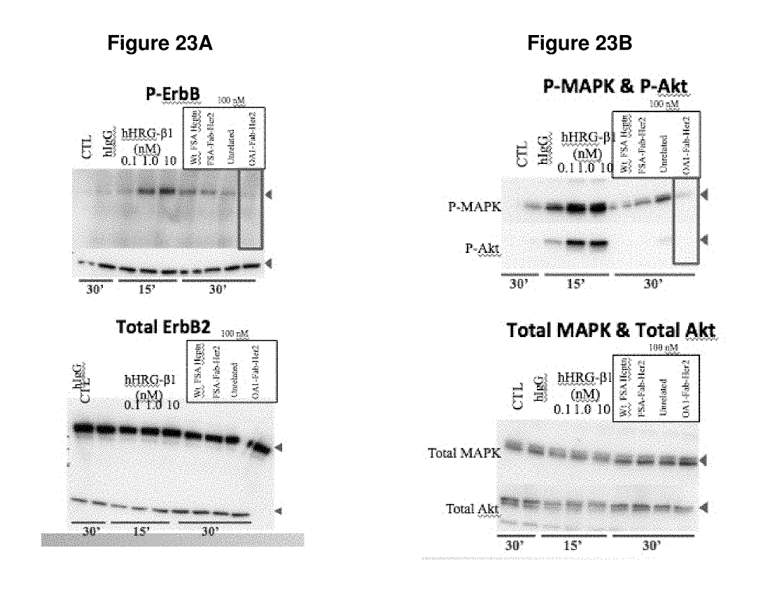

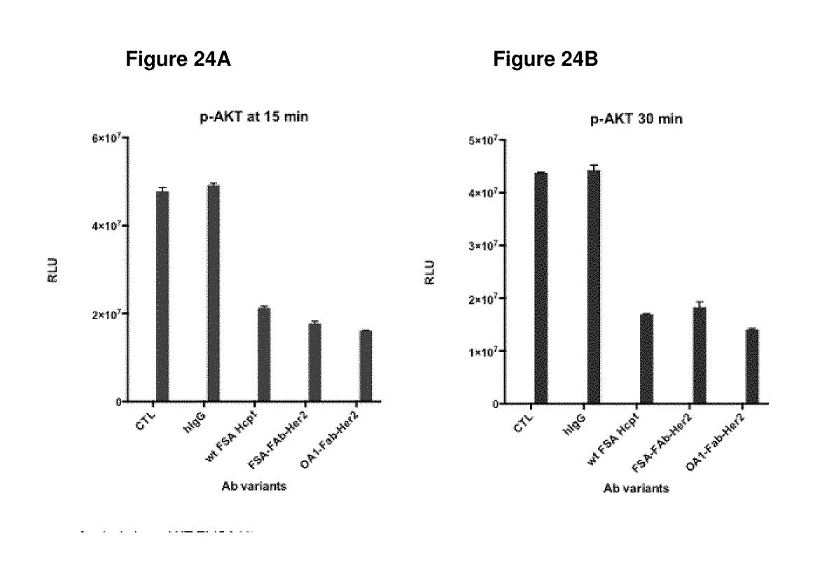

FIGS. 23 A and B show the results with respect to phosphorylation of ErbB, MAPK, and Akt. These results indicate that OA1-Fab-Her2 treatment reduces the amount of total p-MAPk and p-ErbB2 relative to the hIgG control. Of the three anti-Her2 antibodies tested, the greatest reduction in p-MAPk and p-ErbB2 is seen with the OA1-Fab-Her2. Quantitative assessment of the degree of phosphorylation of Akt as measured by ELISA is shown in FIGS. 24 A and B. These results indicate that OA1-Fab-Her2 treatment reduces the amount of total p-AKT relative to the non-treated control (‘CTL’) and hIgG control. Of the three anti-Her2 antibodies tested, the greatest reduction in p-AKT is seen with the OA1-Fab-Her2.

### Example 15: Monovalent Anti-Her2 Antibodies Show Increased Binding to CD16a and CD32a/b Compared to Bivalent Anti-Her2 Antibodies

The ability of the exemplary monovalent anti-Her2 antibodies to bind to FcγRs CD16a and CD32a/b was examined using Surface Plasmon Resonance (SPR).

Surface Plasmon Resonance Analysis: Affinity of FcγRs to antibody Fc was measured by SPR (surface Plasmon resonance) using a ProteOn XPR36 system from BIO-RAD. HER-2 in buffer (10 mM Hepes pH 6.8) was immobilized on CMS chip through amine coupling until 3000 RU. Fc variants in an antibody format containing anti HER2 F(ab)2 were immobilized to the HER-2 surface to 300 RU. Running buffer and the surfactant was maintained at pH 6.8. Purified analyte FcR was diluted in its running buffer and injected at a flow rate of 20-30 mul/min for 2 minutes, followed by dissociation for another 4 minutes. Five twofold dilutions of each antibody beginning at 20 nM were analyzed in triplicate. Sensograms were fit globally to a 1:1 Langmuir binding model. All experiments were conducted at room temperature.

The results of the SPR binding studies are shown in Table 19.

TABLE 19
Binding capacity of monovalent anti-Her2 antibodies

| | Rₘₐₓ | | K_{D} |
|---|---|---|---|
| | fold diff. OA | K_{D} | fold diff. vs. com. |
| FcγR | vs. FSA | (μM) | Herceptin |
| CD16aWT | 1.5 | 48.9 | 1.0 |
| CD16aV158 | 1.7 | 9.38 | 1.0 |
| CD32aWT | 1.6 | 25.6 | 2.1⇓ |
| CD32aR131 | 1.7 | 29.2 | 2.0⇓ |
| CD32bWT | 1.7 | 25.4 | 1.7⇓ |
| CD32bY163 | 1.7 | 96.3 | 1.4⇓ |
| CD64 | 1.8 | 2.6 | 1.0 |

The results in Table 19 indicate that OA1-Fab-Her2 displays a higher Rmax in binding to the FcγRs, compared to the control FSA-Fab-Her2, due to the greater number of Fc regions available for binding to the antigen (Her2) immobilized antibody. Moreover, OA1-Fab-Her2 displays a 1.4-2.0-fold increased affinity for CD32.

### Example 16: Preparation and Expression of Additional Constructs

In addition to constructs 1 to 8 described as in Example 1, the following additional monovalent anti-Her2 antibodies and controls were prepared and tested:

9. OA5-Fab-Her2, a monovalent anti-Her2 antibody, where the Her2 binding domain is a Fab on chain A, and the Fc region is a heterodimer having the mutations T350V_L351Y_F405A_Y407V in Chain A, and T350V_T366L_K92L_T394W in Chain B; the epitope of the antigen binding domain is domain 2 of Her2.
10. OA6-Fab-Her2, a monovalent anti-Her2 antibody, where the Her2 binding domain is a Fab on chain B, and the Fc region is a heterodimer having the mutations T350V_L351Y_F405A_Y407V in Chain A, and T350V_T366L_K92L_T394W in Chain B; the epitope of the antigen binding domain is domain 2 of Her2.
11. FSA-Fab-Pert, a bivalent anti-Her2 antibody, where both Her2 binding domains are pertuzumab in the Fab format, and the Fc region is a heterodimer having the mutations L351Y_S400E_F405A_Y407V in Chain A, and T366I_N390R_K392M_T394W in Chain B. The epitope of the antigen binding domain is domain 2 of Her2.

These constructs were prepared and expressed according to the methods described in Example 1.

### Example 17: Purification of Monovalent Anti-Her2 Antibodies OA5-Fab-Her2 and OA6-Fab-Her2

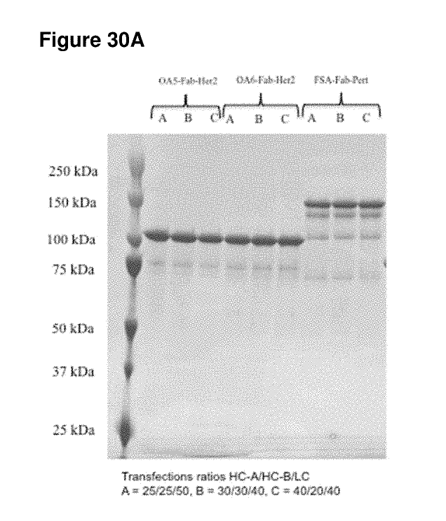

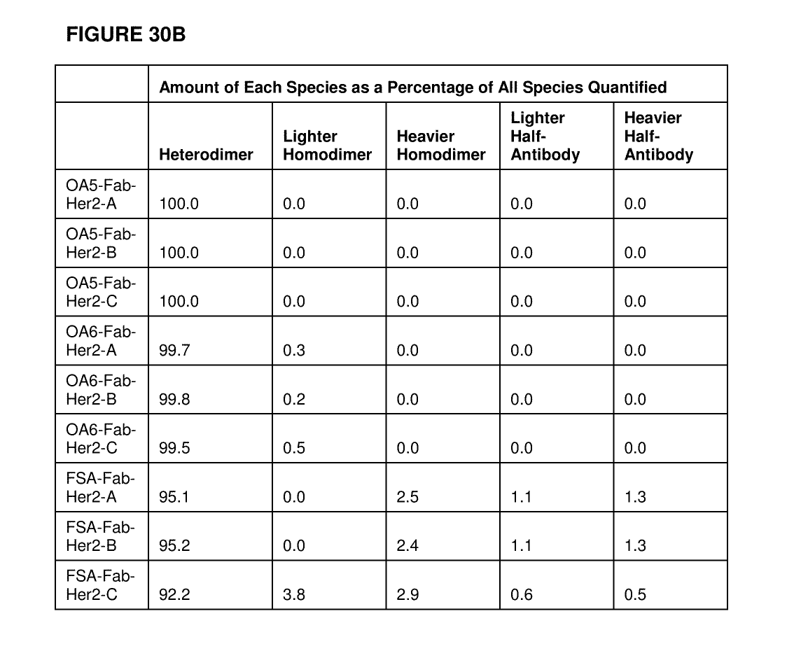

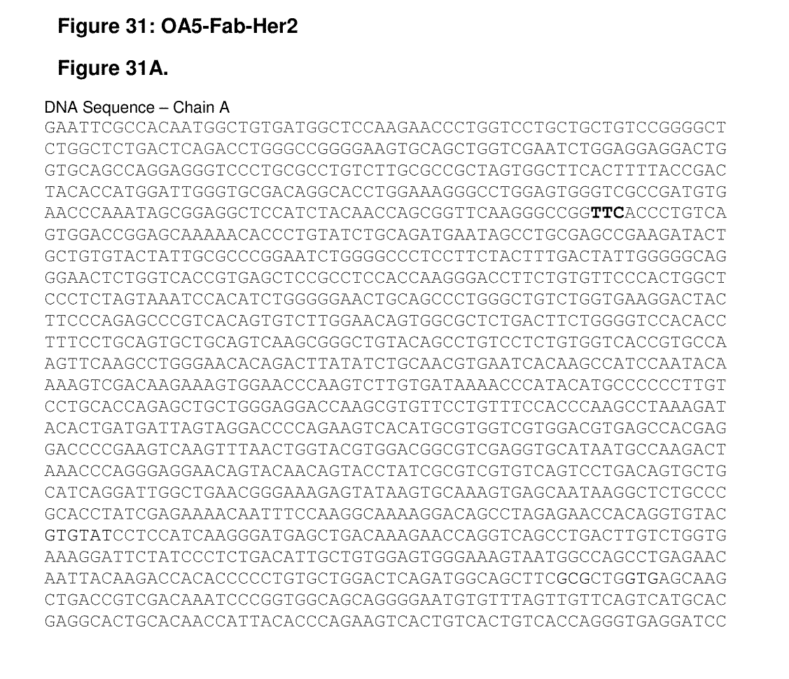

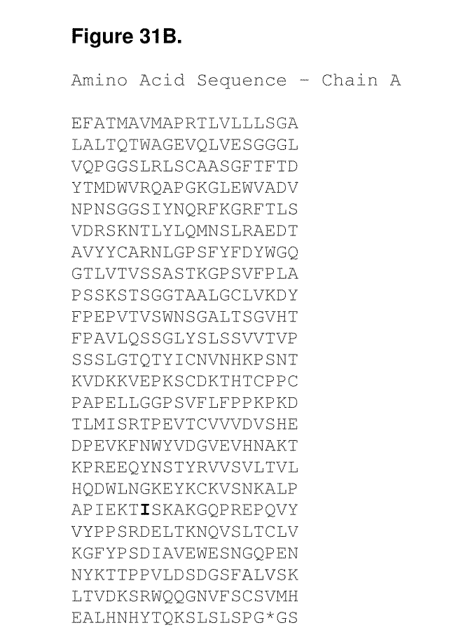

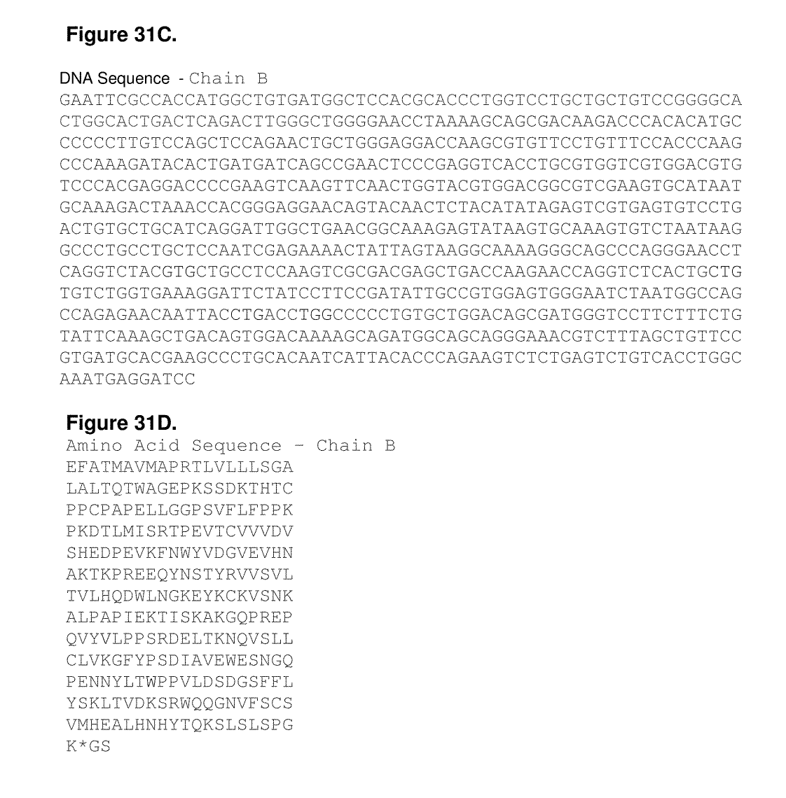

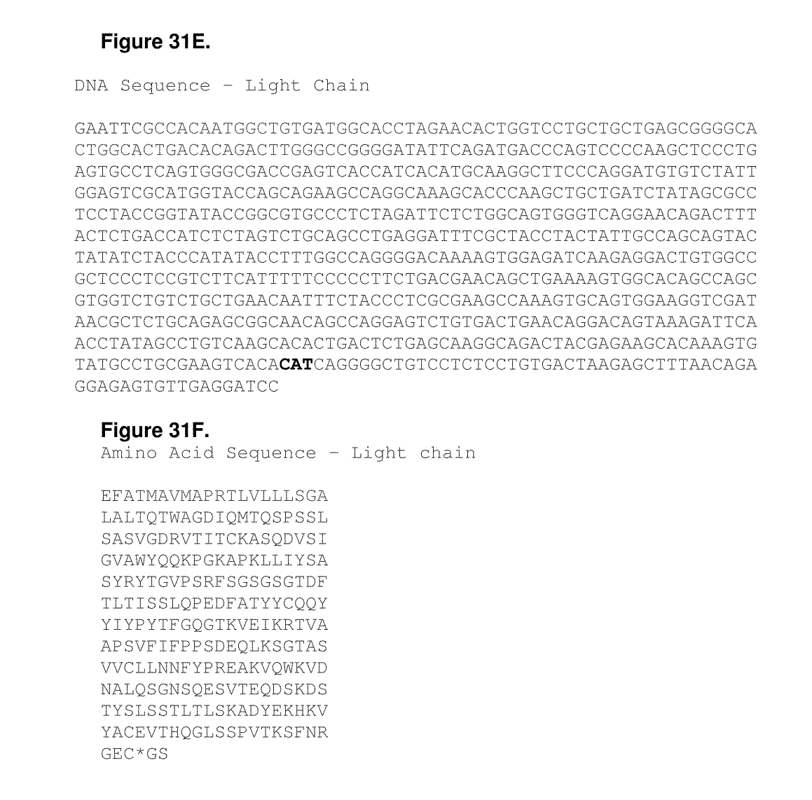

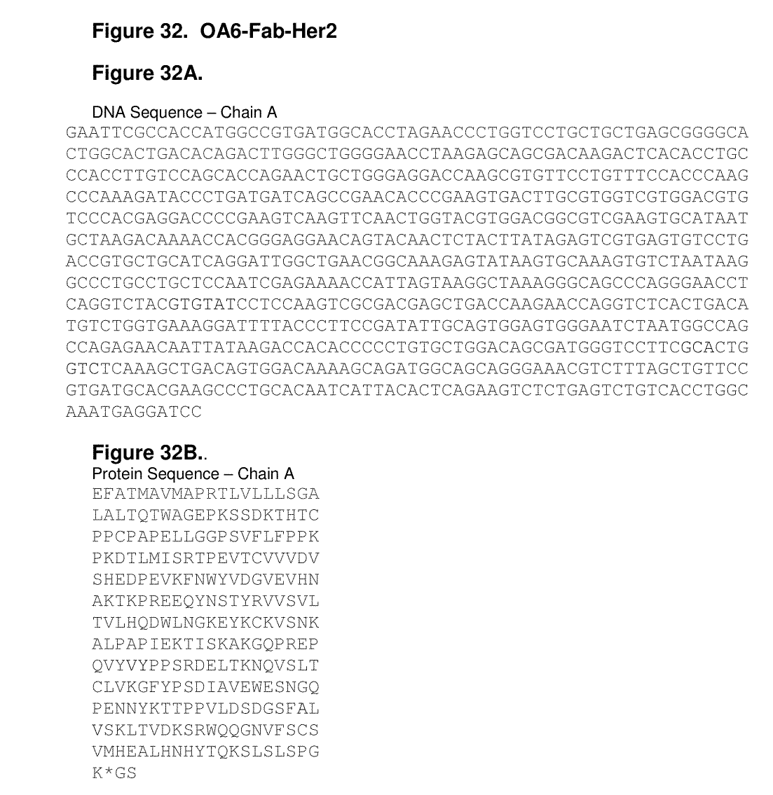

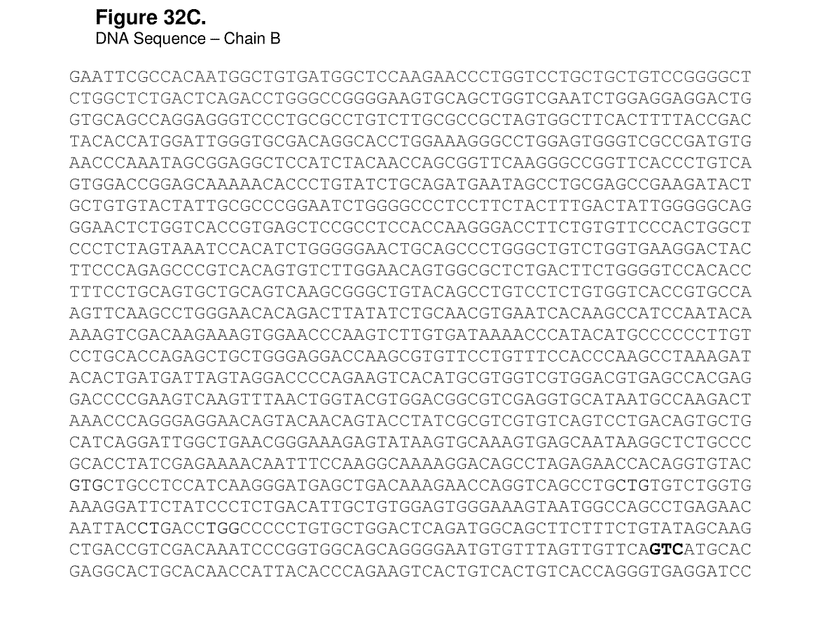

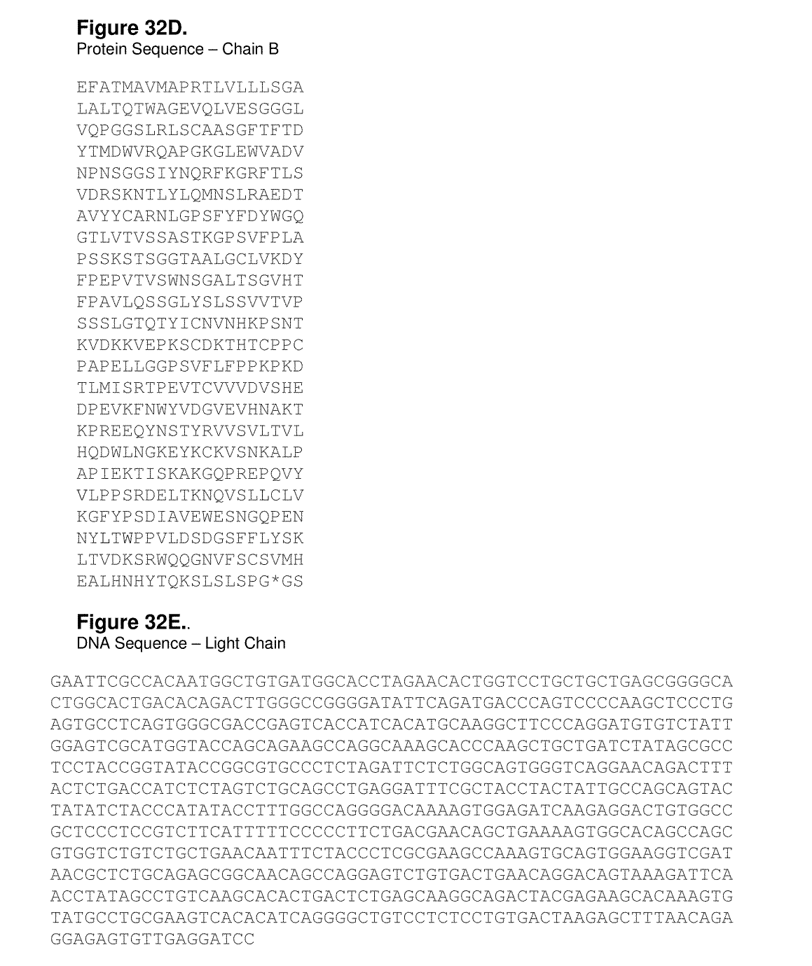

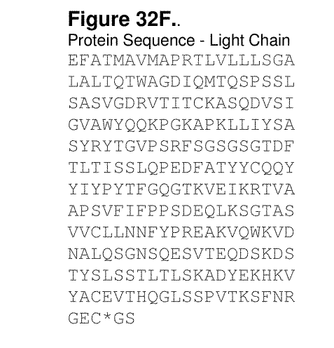

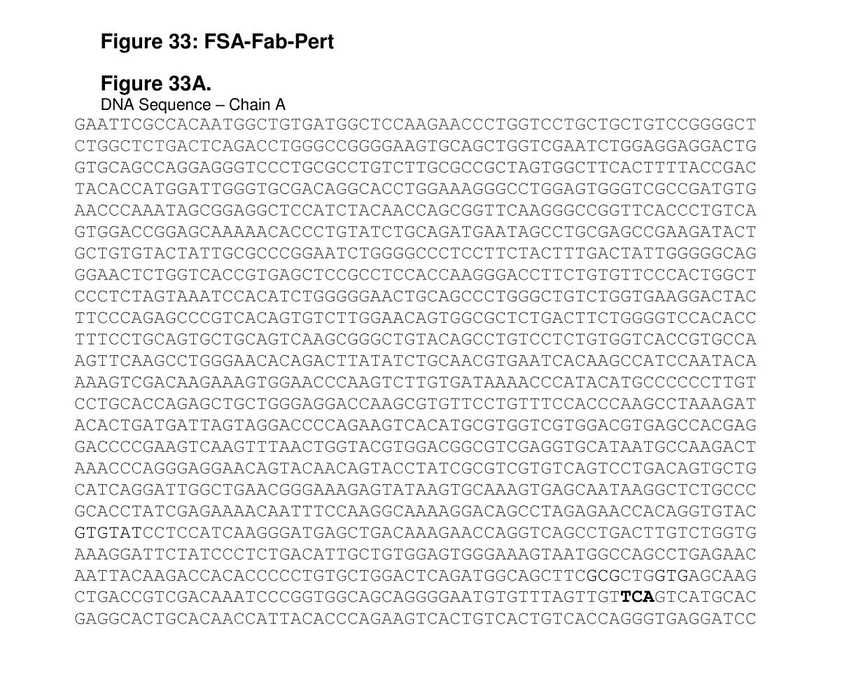

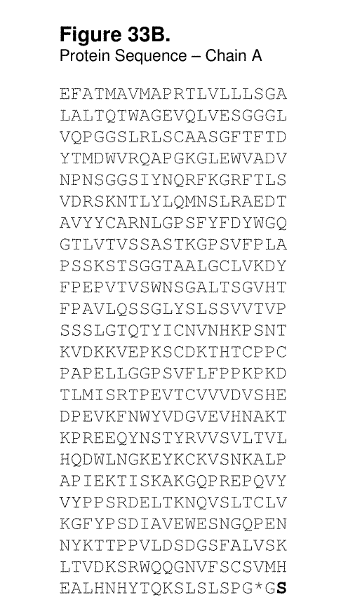

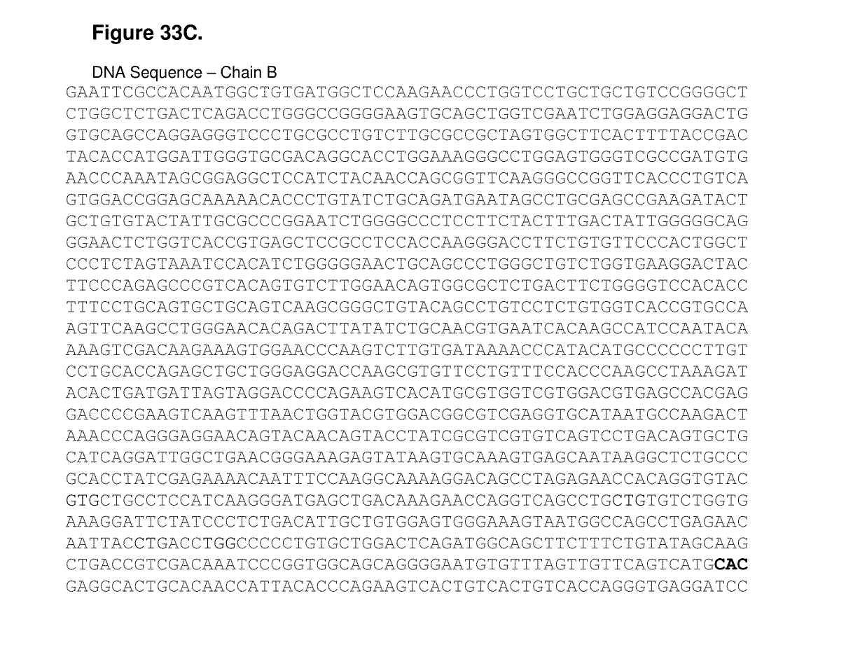

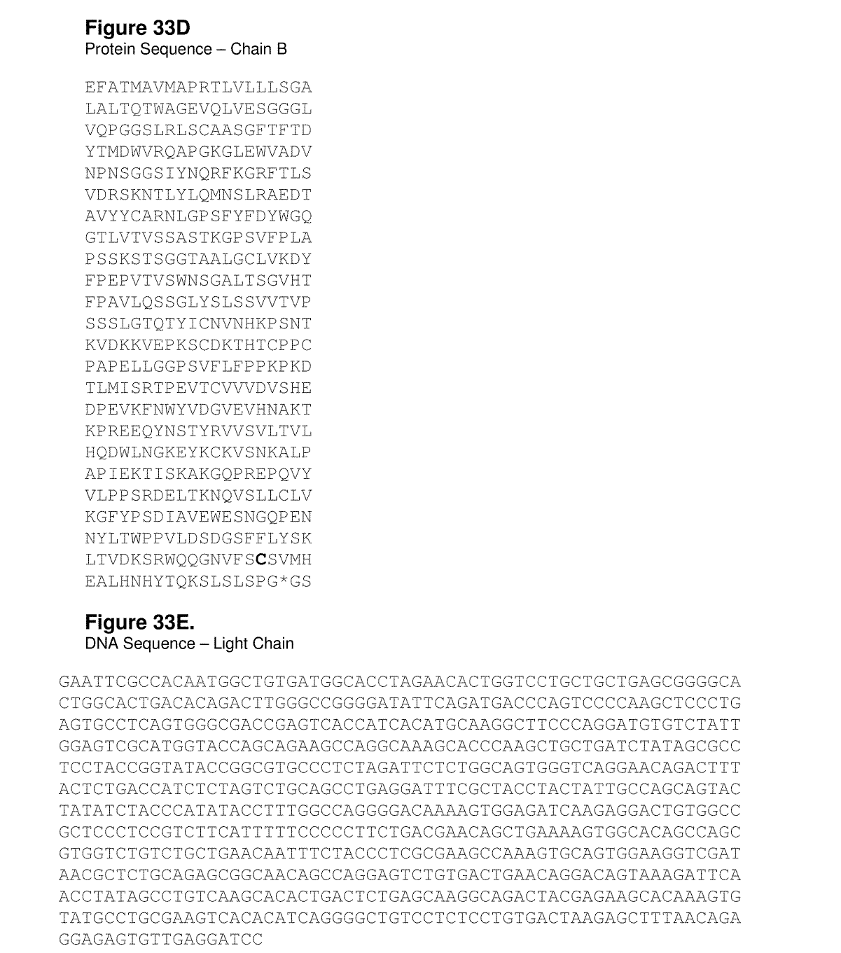

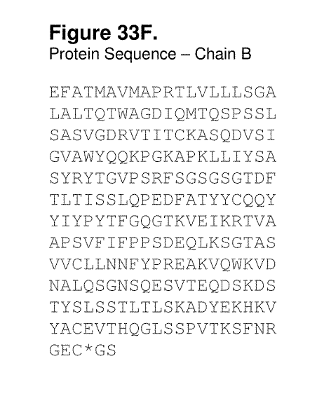

These constructs were prepared and expressed according to the methods described in Example 1. FIG. 30A shows the purity of OA5-Fab-Her2 and OA6-Fab-Her2 post protein A purification. FIG. 30 B shows 5 heterodimer purity analysis by LC/MS which indicates that both OA5-Fab-Her2 and OA6-Fab-Her2 can be purified to greater than 99% purity post protein A and size exclusion chromatography. Heterodimer purity was performed according to the methods described in Example 8.

### Example 18: Monovalent Anti-Her2 Antibodies (Fabs) have a Higher Bₘₐₓ vs. FSA in JIMT-1 and BT-474 Cells

The binding of exemplary monovalent anti-Her2 antibodies (OA5-Fab-Her2 and OA6-Fab-Her2) was compared to that of the bivalent version of these anti-Her2 antibodies (FSA-Fab-pert) in the Her2-expressing cell lines, JIMT-1 and BT-474. These cell lines are used in xenograft models to test the efficacy of candidate anti-cancer therapeutics. The JIMT-1 cell line expresses the Her2 receptor at the 2+ level, and is thus considered to express the receptor with a medium density per cell. The BT-474 cell line is a herceptin-resistant cell line and expresses the Her2 receptor at the 3+ level, and is thus considered to express the receptor with a high density per cell. The monovalent antibodies tested in this example comprise an antibody-binding region that is a Fab. The ability of these antibodies to bind to the surface of these cells was determined by flow cytometry as described in Example 3, with the exception that DMEM containing 10% FBS media was used for the culturing the JIMT-1 cells and the BT-474 cells.

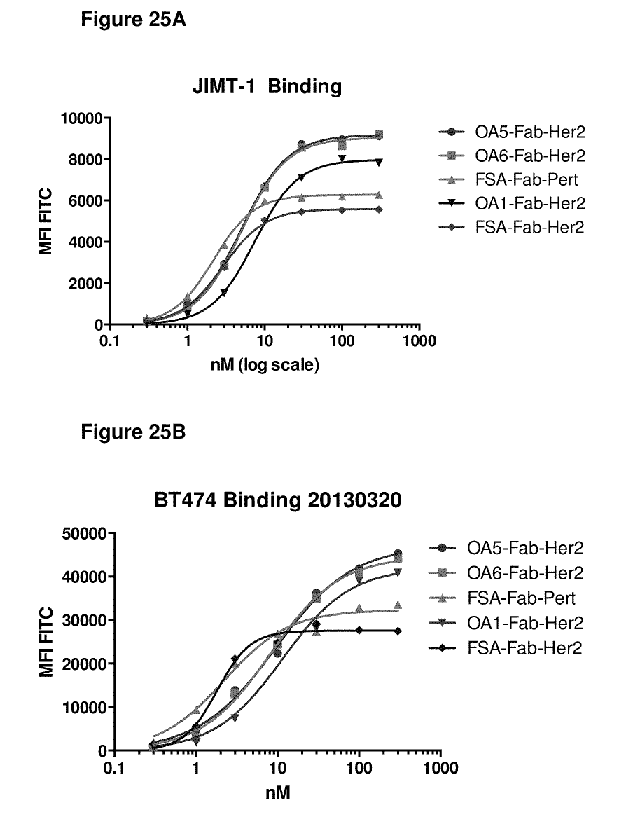

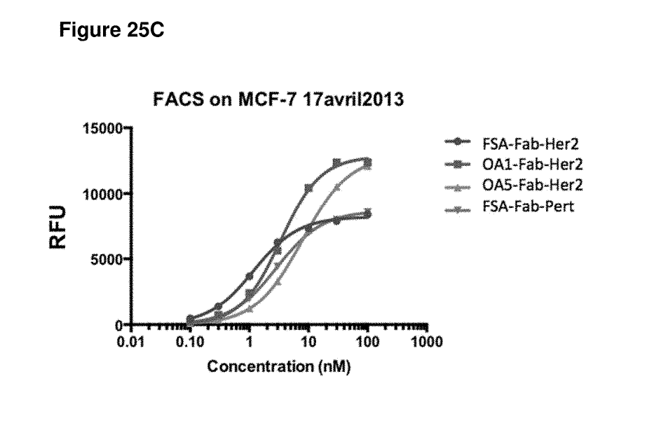

The results are depicted in FIG. 25 A (JIMT-1 cells) and FIG. 25 B (BT-474 cells), and values for K_{D} and Bₘₐₓ are shown in Tables 20 and 21 below.

TABLE 20
Binding data in JIMT-1 cells

| Antibody | K_{D} (nM) | Bₘₐₓ (MFI) |
|---|---|---|
| OA1-Fab-Her2 | 7.39 | 7969 |
| FSA-Fab-Her2 | 2.87 | 5585 |
| OA5-Fab-Her2 | 4.96 | 9172 |
| OA6-Fab-Her2 | 5.01 | 9031 |
| FSA-Fab-Pert | 2.19 | 6271 |

The data shown in FIG. 25A and Table 20 show that the fold difference in K_{D} for OA1-Fab-Her2 vs. FSA-Fab-Her2 is 2.57 (increase), while the fold difference in Bₘₐₓ for OA1-Fab-Her2 vs. FSA-Fab-Her2 is 1.43 (increase). The fold difference in K_{D} for OA5-Fab-Her2 vs. FSA-Fab-pert is 2.26 (increase), while the fold difference in Bₘₐₓ for OA5-Fab-Her2 vs. FSA-Fab-pert is 1.46 (increase).

TABLE 21
Binding data for BT-474 cells

| Variant | K_{D} (nM) | Bₘₐₓ (MFI) |
|---|---|---|
| OA1-Fab-Her2 | 11.5 | 42033 |
| FSA-Fab-Her2 | 1.81 | 27548 |
| OA5-Fab-Her2 | 9.47 | 47072 |
| OA6-Fab-Her2 | 8.20 | 44578 |
| FSA-Fab-Pert | 2.22 | 32295 |

The data shown in FIG. 25B and Table 21 show that the fold difference in K_{D} for OA1-Fab-Her2 vs. FSA-Fab-Her2 is 6.35 (increase), while the fold difference in Bₘₐₓ for OA1-Fab-Her2 vs. FSA-Fab-Her2 is 1.52 (increase). The fold difference in K_{D} for OA5-Fab-Her2 vs. FSA-Fab-pert is 4.66 (increase), while the fold difference in Bₘₐₓ for OA5-Fab-Her2 vs. FSA-Fab-pert is 1.45 (increase).

In summary, in both cell types tested in this example the monovalent anti-Her2 antibodies tested have a higher Bₘₐₓ compared to the relevant bivalent control antibodies. These results also indicate that the monovalent anti-Her2 antibodies based on pertuzumab (OA5-Fab-Her2 and OA6-Fab-Her2) have a higher Bₘₐₓ that those based on herceptin (OA1-Fab-Her2).

### Example 19: Monovalent Anti-Her2 Antibodies Inhibit Growth of BT-474 Cells

The ability of monovalent anti-Her2 antibodies to inhibit the growth of BT-474 cells and JIMT-1 cells, grown in DMEM containing 10% FBS, was tested using the method described in Example 9.

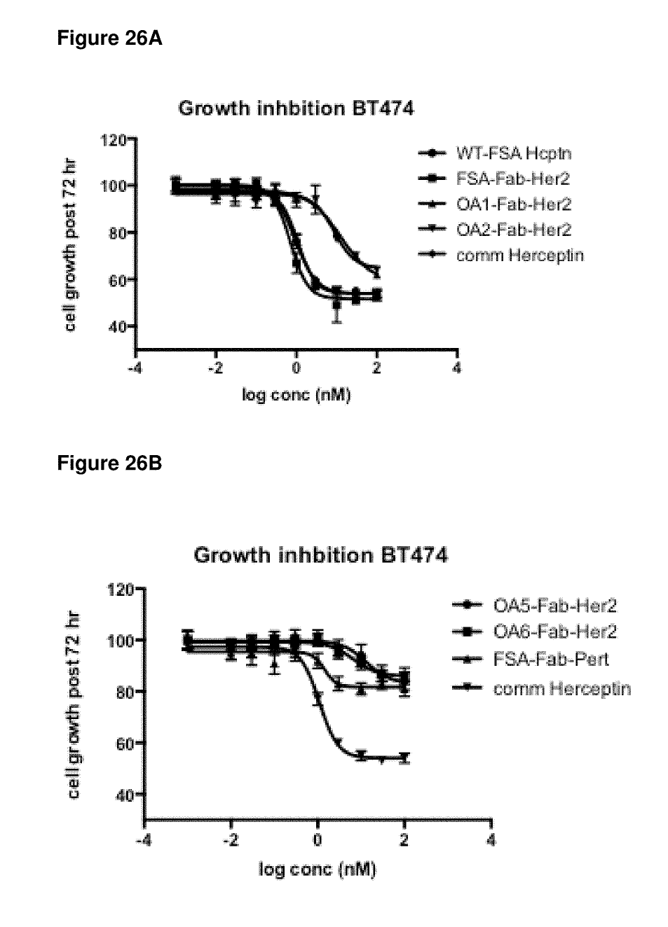

The results for BT-474 cells are shown in FIGS. 26A and B and the % maximum growth inhibition for the antibodies tested is shown in Table 22.

TABLE 22
Maximum growth inhibition

| | % Max Growth |
|---|---|
| Variant | Inhibition |
| Com. Hcptn | 46 |
| Wt FSA Hcptn | 46 |
| FSA-Fab-Her2 | 48 |
| OA1-Fab-Her2 | 41 |
| OA2-Fab-Her2 | 35 |
| FSA-Fab-Pert | 17 |
| OA5-Fab-Her2 | 14 |
| OA6-Fab-Her2 | 18 |

None of the antibodies tested (FSA-Fab-Her2, wt FSA Hcptn, OA1-Fab-Her2, OA2-Fab-Her2, OA5-Fab-Her2, OA6-Fab-Her2, FSA-Fab-pert, or commercial Herceptin™ were able to inhibit the growth of JIMT-1 cells (data not shown).

### Example 20: Monovalent Anti-Her2 Antibodies are Internalized

The ability of exemplary monovalent anti-Her2 antibodies to be internalized by BT-474 cells was determined using a “direct” method distinct from the “indirect” method used in Example 9.

The direct internalization method was followed according to the protocol detailed in Schmidt, M. et al., Kinetics of anti-carcinoembryonic antigen antibody internalization: effects of affinity, bivalency, and stability. Cancer Immunol Immunother (2008) 57:1879-1890. Specifically, the antibodies were directly labeled using the AlexaFluor® 488 Protein Labeling Kit (Invitrogen, cat. no. A10235), according to the manufacturer's instructions.

For the internalization assay, 12 well plates were seeded with 1×10⁵ cells/well and incubated overnight at 37° C.+5% CO₂. The following day, the labeled antibodies were added at 10 and 200 nM in DMEM+10% FBS and incubated 24 hours at 37° C.+5% CO₂. Under dark conditions, media was aspirated and wells were washed 2×500 pt PBS. To harvest cells, cell dissociation buffer was added (250 μL) at 37° C. Cells were pelleted and resuspended in 100 pt DMEM+10% FBS without or with anti-Alexa Fluor 488, rabbit IgG fraction (Molecular Probes, A11094, lot 1214711) at 50 μg/mL, and incubated on ice for 30 min. Prior to analysis 300 DMEM+10% FBS the samples filtered 4 ul propidium iodide was added. Samples were analyzed using the LSRII flow cytometer.

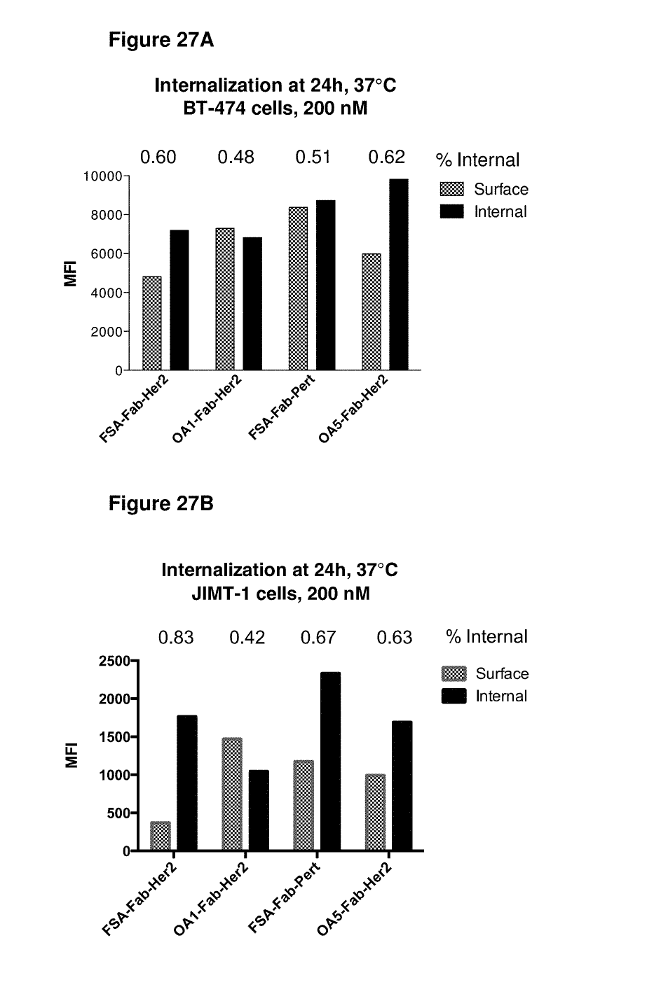

The results are shown in FIGS. 27A and B. FIG. 27A illustrates that both OA1-Fab-Her2 and OA5-Fab-Her2 (at 200 nM) are capable on internalizing in BT-474 cells at a percentage that is comparable to the parent FSA antibody. In the case of OA5-Fab-Her2, higher total internalization is seen with the OA (62%) compared to the it's FSA, FSA-Fab-Pert (51%). FIG. 27B illustrates that both OA1-Fab-Her2 and OA5-Fab-Her2 (at 200 nM) are capable on internalizing in JIMT-1 (herceptin resistant) cells at a percentage that is comparable to the parent FSA antibody. In BT-474 and JIMT-1, OA5-Fab-Her2, has a higher % internalization compared to ° A1-Fab-Her2.

### Example 21: Monovalent Anti-Her2 Antibodies Show Increased ADCC in Her2 1+ Cell Line (MCF7 Cells)

In addition to the exemplary monovalent anti-Her2 antibody tested in (OA1-Fab-Her2), the ability of additional monovalent anti-HER2 antibodies OA4-scFv-BID2, OA5-Fab-Her2 and OA6-Fab-Her2 to mediate ADCC compared to the relevant control FSA antibodies was tested. Additional controls included the commercial Herceptin™ antibody, wt FSA Hcptn and FSA-Fab-Her2. ADCC activity was measured in the Her2 1+ cell line MCF7 according to the protocol described in Examples 5 and 12.

The results are shown in FIGS. 21 C, D, and E. FIG. 21 C shows a comparison of OA1-Fab-Her2, OA4-scFv-BID2 and OA5-Fab-Her2 in an ADCC assay in MCF-7 (Her2 1+) cells. The results in FIG. 21C show that treatment with OA1-Fab-Her2 mediates the greatest maximum target cell lysis and that this maximum target cell lysis is greater than that of Commercial Herceptin. Commercial Herceptin has ca. 18% less core fucose residues; the absence of, or reduction in, core fucose is known to enhance in vitro target cell lysis (by ADCC), compared to fucosylated antibodies (Suzuki E. et al. 2007, A non-fucosylated anti-HER2 antibody augments antibody-dependent cellular cytotoxicity in breast cancer patients Clin Cancer Res. 13:1875-1882). Despite OA1-Fab-Her2 possessing a greater percentage of fucosylated peptide sequences relative to Commercial Herceptin, it is able to mediate greater target cell lysis. The results in FIG. 21D compare the FSA anti-Her2 variants and show a reduced maximum target cell lysis relative to the Commercial Herceptin. Comparing Commercial Herceptin with FSA-Fab-Her2 (identical molecules with exception of differences in fucosylation) illustrates the large effect imparted by the glycosylation. The results in FIG. 21 E show the superior killing mediated by OA1-Fab-Her2 compared to the parent FSA antibody, FSA-Fab-Her2, and compared to Commercial Herceptin. Of the three OA anti-Her2 antibodies, OA1-Fab-Her2 mediates the greatest % of target cell lysis in MCF-7 cells.

### Example 22: Monovalent Anti-Her2 Antibodies (scFvs) have a Higher Bₘₐₓ Vs. FSA in MALME-3M Cells

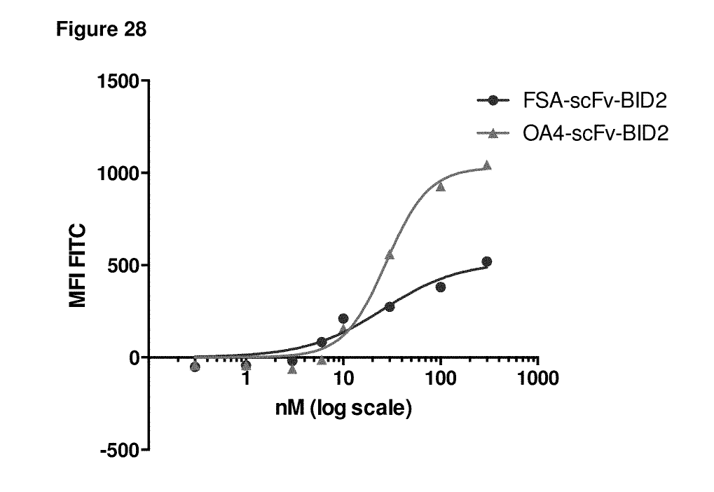

The binding of the exemplary monovalent anti-HER2 OA4-scFv-BID2 was compared to that of the bivalent version of this anti-Her2 antibody FSA-scFv-BID2 in MALME-3M cells. The assay was carried out by flow cytometry as described in Example 3. The results are shown in FIG. 28. The data indicates that OA4-scFv-BID2 displays superior binding to MALME-3M cells compared to the FSA-scFv-BID2 antibody.

### Example 23: Ability of Monovalent Antibody Construct-ADC to Kill Cells

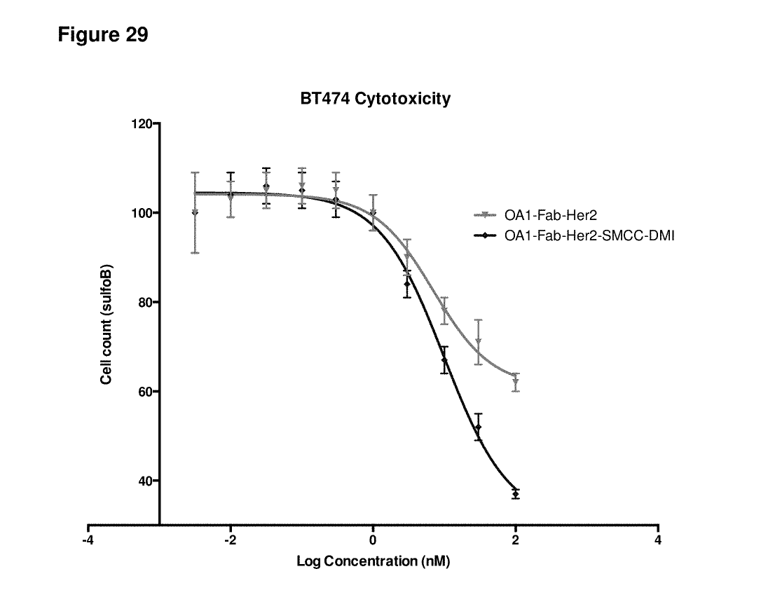

A monovalent antibody construct OA1-Fab-Her2 conjugated to a toxic drug molecule (OA-Fab-MCC-DM1) was prepared as follows: Antibody-drug conjugates were prepared using or N-succinimidyl-4-(N-maleimidomethyl)cyclohexane-1-carboxylate (SMCC) for thioether linkage as described in Chari et al. 1992, Immunoconjugates containing novel maytansinoids: promising anti-cancer drugs. Cancer Res 1992: 52:127-31. The ability of this molecule growth inhibit BT474 cells was tested using the method described in Example 9. The results are shown in FIG. 29 and indicate that following 72 h treatment, the OA1-Fab-Her2-MCC-DM 1 resulted in 63% growth inhibition in BT-474 at 100 nM compared to 38% growth inhibition with OA1-FSA-Her2. This data indicated that the OA-Fab-MCC-DM1 displays superior growth inhibition compared to OA1-Fab-Her2.

### Example 24: Determination of Binding Kinetics and Affinity for an Exemplary Monovalent Antibody Construct

The binding kinetics and affinity of OA2-Fab-Her2 for HER2 were determined by SPR as follows using a ProteOn XPR36 system from BIO-RAD. Approximately 3300 RU of anti-human IgG 25 ug/ml was immobilized on a GLC chip using standard amine coupling. Wt FSA Hcptn or OA2-Fab-Her2 (20 ug/ml in PBST, 25 ul/min) was captured on the anti-human IgG immobilized chip to capture level of approximately 700 RU. Recombinant human HER2 was diluted in PBST at 60, 20, 6.66, 2.22, 0.74 nM and injected at a flow rate of 50 μl/min for 2 minutes, followed by dissociation for another 4 minutes. HER2 dilutions were analyzed in triplicate. Sensograms were fit globally to a 1:1 Langmuir binding model. All experiments were conducted at room temperature.

The results are shown in Table 23 below and provide measurements for kₐ (on-rate, kinetic association rate), k_{d} (off-rate, kinetic dissociation rate), and K_{D} (equilibrium dissociation constant).

TABLE 23
Summary of binding kinetics and affinity for OA2-Fab-HER2 compared
to the corresponding monospecific bivalent antibody construct.

| | | | | K_{D} | |
|---|---|---|---|---|---|
| Antibody | | kₐ (M−1s−1) | k_{d} (s−1) | (M) | n |
| Wt FSA | Average | 3.91E+05 | 1.06E−04 | 2.83E−10 | 4 |
| Hcptn | Stdev | 77975.9 | 9.47E−06 | 8.38E−11 | |
| OA2-Fab- | Average | 3.13E+05 | 1.31E−04 | 4.35E−10 | 4 |
| Her2 | Stdev | 63489.5 | 9.67E−06 | 1.14E−10 | |

These results indicate that the on-rate, off-rate, and equilibrium dissociation constant for the exemplary monovalent antibody construct tested are comparable to that of the corresponding monospecific bivalent antibody construct.

The reagents employed in the examples are commercially available or can be prepared using commercially available instrumentation, methods, or reagents known in the art. The foregoing examples illustrate various aspects of the invention and practice of the methods of the invention. The examples are not intended to provide an exhaustive description of the many different embodiments of the invention. Thus, although the forgoing invention has been described in some detail by way of illustration and example for purposes of clarity of understanding, those of ordinary skill in the art will realize readily that many changes and modifications can be made thereto without departing from the spirit or scope of the appended claims.

All publications, patents and patent applications mentioned in this specification are herein incorporated by reference into the specification to the same extent as if each individual publication, patent or patent application was specifically and individually indicated to be incorporated herein by reference.

1. An isolated antibody construct that binds human epidermal growth factor receptor 2 (HER2) comprising:
a single antigen binding polypeptide construct linked to an N-terminus of a dimeric Fc polypeptide construct comprising two monomeric Fc polypeptides each comprising a CH3 domain, wherein the single antigen binding polypeptide construct is a Fab or an scFv that binds to one of domain 1, 2, or 4 of HER2;
wherein the antibody construct displays an increase in Bₘₐₓ to HER2 displayed on the surface of a target cell as compared to a corresponding and otherwise identical antibody construct, except that it comprises two antigen binding polypeptide constructs, at equimolar concentrations above K_{D} and at saturation.

2. The isolated antibody construct of claim 1 wherein the antibody construct is conjugated to one or more drug molecules.

3. The isolated antibody construct of claim 1 wherein the single antigen binding polypeptide construct is a Fab.

4. The isolated antibody construct of claim 1, wherein the increase in Bₘₐₓ is at least about 125% of the Bₘₐₓ of the corresponding antibody construct.

5. The isolated antibody construct according to claim 1, wherein the dimeric Fc polypeptide construct is a heterodimeric Fc construct having a variant CH3 domain comprising amino acid mutations that promote the formation of the heterodimer with stability comparable to a native homodimeric Fc region.

6. The isolated antibody construct of claim 5, wherein the variant CH3 domain has a melting temperature (Tm) of about 70° C. or higher.

7. The isolated antibody construct of claim 1, wherein the dimeric Fc polypeptide construct further comprises a variant CH2 domain comprising amino acid modifications to promote selective binding of Fcgamma receptors.

8. The isolated antibody construct according to claim 1, wherein one monomeric Fc polypeptide is linked to the single antigen-binding polypeptide construct by a linker.

9. A host cell comprising one or more nucleic acids encoding the isolated antibody construct according to claim 1.

10. A method of preparing the isolated antibody construct according to claim 1, the method comprising the steps of: (a) culturing a host cell comprising nucleic acid encoding the antibody construct; and (b) recovering the antibody construct from the host cell culture.

11. A pharmaceutical composition comprising the antibody construct according to claim 1 and a pharmaceutically acceptable carrier.

12. A method of treating cancer comprising providing to a patient in need thereof an effective amount of the antibody construct of claim 1.

13. A method of inhibiting growth of a tumor, shrinking a tumor, or killing a tumor comprising contacting the tumor with an effective amount of the antibody construct according to claim 1.

14. The isolated antibody construct of claim 1, wherein the antigen-binding polypeptide construct is afucosylated or glycosylated.

15. The isolated antibody construct of claim 5, wherein
1. one monomeric Fc polypeptide comprises the mutations T350V_L351Y_F405A_Y407V and the other monomeric Fc polypeptide comprises the mutations T350V_T366L_K392L_T394W;
2. one monomeric Fc polypeptide comprises the mutations T350V_L351Y_F405A_Y407V and the other monomeric Fc polypeptide comprises the mutations T350V_T366L_K392M_T394W;
3. one monomeric Fc polypeptide comprises the mutations L351Y_F405A_Y407V and the other monomeric Fc polypeptide comprises the mutations T366L_K392L_T394W;
4. one monomeric Fc polypeptide comprises the mutations L351Y_F405A_Y407V and the other monomeric Fc polypeptide comprises the mutations T366L_K392M_T394W; or
5. one monomeric Fc polypeptide comprises the mutations L351Y_S400E_F405A_Y407V and the other monomeric Fc polypeptide comprises the mutations T366I_N390R_K392M_T394W,
and wherein the numbering is according to the EU index according to Kabat.

16. The isolated antibody construct of claim 1, wherein the antibody construct comprises:
a. the final protein product sequence as set forth in SEQ ID NO: 14, without the signal peptide sequence; the final protein product sequence as set forth in SEQ ID NO: 12, without the signal peptide sequence; and the final protein product sequence as set forth in SEQ ID NO: 16, without the signal peptide sequence;
b. the final protein product sequence as set forth in SEQ ID NO: 22, without the signal peptide sequence; the final protein product sequence as set forth in SEQ ID NO: 18, without the signal peptide sequence; and the final protein product sequence as set forth in SEQ ID NO: 20, without the signal peptide sequence;
c. the final protein product sequence as set forth in SEQ ID NO: 44, without the signal peptide sequence; the final protein product sequence as set forth in SEQ ID NO: 40, without the signal peptide sequence; and the final protein product sequence as set forth in SEQ ID NO: 42, without the signal peptide sequence;
d. the final protein product sequence as set forth in SEQ ID NO: 50, without the signal peptide sequence; the final protein product sequence as set forth in SEQ ID NO: 46, without the signal peptide sequence; and the final protein product sequence as set forth in SEQ ID NO: 48, without the signal peptide sequence; or
e. the final protein product sequence as set forth in SEQ ID NO: 36, without the signal peptide sequence; and the final protein product sequence as set forth in SEQ ID NO: 38, without the signal peptide sequence.

17. A vector comprising one or more nucleic acids encoding the isolated antibody construct of claim 1.

18. One or more nucleic acids encoding the isolated antibody construct of claim 1.

19. The isolated antibody construct of claim 1, wherein the single antigen binding polypeptide construct is an scFv.

20. A method of treating breast cancer comprising providing to a patient in need thereof an effective amount of the antibody construct of claim 1.


SEQUENCE LISTING (56 sequences)

SEQ ID NO: 1 - protein, 17 aa, Homo sapiens
MLQNSAVLLLLVISASA

SEQ ID NO: 2 - protein, 22 aa, Artificial Sequence
  Description of Artificial Sequence Synthetic peptide
MPTWAWWLFLVLLLALWAPARG

SEQ ID NO: 3 - DNA, 1539 bp, Homo sapiens
gaattcgccaccatggccgtgatggctcctagaaccctggtgctgctgctgtctggagctctggctctgactcagacctgggctggagacatccagatgacccagtctccatcctccctgtctgcatctgtaggagacagagtcaccatcacttgccgggcaagtcaggacgttaacaccgctgtagcttggtatcagcagaaaccagggaaagcccctaagctcctgatctattctgcatcctttttgtacagtggggtcccatcaaggttcagtggcagtcgatctgggacagatttcactctcaccatcagcagtctgcaacctgaagattttgcaacttactactgtcaacagcattacactaccccacccactttcggccaagggaccaaagtggagatcaaaggtggttctggtggtggttctggtggtggttctggtggtggttctggtggtggttctggtgaagtgcagctggtggagtctgggggaggcttggtacagcctggcgggtccctgagactctcctgtgcagcctctggattcaacattaaagatacttatatccactgggtccggcaagctccagggaagggcctggagtgggtcgcacgtatttatcccacaaatggttacacacggtatgcggactctgtgaagggccgattcaccatctccgcagacacttccaagaacaccgcgtatctgcaaatgaacagtctgagagctgaggacacggccgtttattactgttcaagatggggcggagacggtttctacgctatggactactggggccaagggaccctggtcaccgtctcctcagccgccgagcccaagagcagcgataagacccacacctgccctccctgtccagctccagaactgctgggaggacctagcgtgttcctgtttccccctaagccaaaagacactctgatgatttccaggactcccgaggtgacctgcgtggtggtggacgtgtctcacgaggaccccgaagtgaagttcaactggtacgtggatggcgtggaagtgcataatgctaagacaaaaccaagagaggaacagtacaactccacttatcgcgtcgtgagcgtgctgaccgtgctgcaccaggactggctgaacgggaaggagtataagtgcaaagtcagtaataaggccctgcctgctccaatcgaaaaaaccatctctaaggccaaaggccagccaagggagccccaggtgtacacatacccacccagcagagacgaactgaccaagaaccaggtgtccctgacatgtctggtgaaaggcttctatcctagtgatattgctgtggagtgggaatcaaatggacagccagagaacaattacaagaccacacctccagtgctggacgaggatggcagcttcgccctggtgtccaagctgacagtggataaatctcgatggcagcaggggaacgtgtttagttgttcagtgatgcatgaagccctgcacaatcattacactcagaagagcctgtccctgtctcccggcaaatgaggatcc

SEQ ID NO: 4 - protein, 510 aa, Homo sapiens
  misc_feature (30)..(510): Final protein product
EFATMAVMAPRTLVLLLSGALALTQTWAGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTYPPSRDELTKNQVSLTCLVKGFYPSDIAVEWESNGQPENNYKTTPPVLDEDGSFALVSKLTVDKSRWQQGNVFSCSVMHEALHNHYTQKSLSLSPGK

SEQ ID NO: 5 - DNA, 1539 bp, Homo sapiens
gaattcgccaccatggccgtgatggctcctagaaccctggtgctgctgctgtctggagctctggctctgactcagacctgggctggagacatccagatgacccagtctccatcctccctgtctgcatctgtaggagacagagtcaccatcacttgccgggcaagtcaggacgttaacaccgctgtagcttggtatcagcagaaaccagggaaagcccctaagctcctgatctattctgcatcctttttgtacagtggggtcccatcaaggttcagtggcagtcgatctgggacagatttcactctcaccatcagcagtctgcaacctgaagattttgcaacttactactgtcaacagcattacactaccccacccactttcggccaagggaccaaagtggagatcaaaggtggttctggtggtggttctggtggtggttctggtggtggttctggtggtggttctggtgaagtgcagctggtggagtctgggggaggcttggtacagcctggcgggtccctgagactctcctgtgcagcctctggattcaacattaaagatacttatatccactgggtccggcaagctccagggaagggcctggagtgggtcgcacgtatttatcccacaaatggttacacacggtatgcggactctgtgaagggccgattcaccatctccgcagacacttccaagaacaccgcgtatctgcaaatgaacagtctgagagctgaggacacggccgtttattactgttcaagatggggcggagacggtttctacgctatggactactggggccaagggaccctggtcaccgtctcctcagccgccgagcccaagagcagcgataagacccacacctgccctccctgtccagctccagaactgctgggaggacctagcgtgttcctgtttccccctaagccaaaagacactctgatgatttccaggactcccgaggtgacctgcgtggtggtggacgtgtctcacgaggaccccgaagtgaagttcaactggtacgtggatggcgtggaagtgcataatgctaagacaaaaccaagagaggaacagtacaactccacttatcgcgtcgtgagcgtgctgaccgtgctgcaccaggactggctgaacgggaaggagtataagtgcaaagtcagtaataaggccctgcctgctccaatcgaaaaaaccatctctaaggccaaaggccagccaagggagccccaggtgtacacactgccacccagcagagacgaactgaccaagaaccaggtgtccctgatctgtctggtgaaaggcttctatcctagtgatattgctgtggagtgggaatcaaatggacagccagagaacagatacatgacctggcctccagtgctggacagcgatggcagcttcttcctgtattccaagctgacagtggataaatctcgatggcagcaggggaacgtgtttagttgttcagtgatgcatgaagccctgcacaatcattacactcagaagagcctgtccctgtctcccggcaaatgaggatcc

SEQ ID NO: 6 - protein, 510 aa, Homo sapiens
  misc_feature (30)..(510): Final protein product
EFATMAVMAPRTLVLLLSGALALTQTWAGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLICLVKGFYPSDIAVEWESNGQPENRYMTWPPVLDSDGSFFLYSKLTVDKSRWQQGNVFSCSVMHEALHNHYTQKSLSLSPGK

SEQ ID NO: 7 - DNA, 1539 bp, Homo sapiens; identical to SEQ ID NO: 3

SEQ ID NO: 8 - protein, 510 aa, Homo sapiens; identical to SEQ ID NO: 4
  misc_feature (30)..(510): Final protein product

SEQ ID NO: 9 - DNA, 792 bp, Homo sapiens
gaattcgccaccatggccgtgatggctcctagaaccctggtgctgctgctgtctggagctctggctctgactcagacctgggctggagagcccaagagcagcgataagacccacacctgccctccctgtccagctccagaactgctgggaggacctagcgtgttcctgtttccccctaagccaaaagacactctgatgatttccaggactcccgaggtgacctgcgtggtggtggacgtgtctcacgaggaccccgaagtgaagttcaactggtacgtggatggcgtggaagtgcataatgctaagacaaaaccaagagaggaacagtacaactccacttatcgcgtcgtgagcgtgctgaccgtgctgcaccaggactggctgaacgggaaggagtataagtgcaaagtcagtaataaggccctgcctgctccaatcgaaaaaaccatctctaaggccaaaggccagccaagggagccccaggtgtacacactgccacccagcagagacgaactgaccaagaaccaggtgtccctgatctgtctggtgaaaggcttctatcctagtgatattgctgtggagtgggaatcaaatggacagccagagaacagatacatgacctggcctccagtgctggacagcgatggcagcttcttcctgtattccaagctgacagtggataaatctcgatggcagcaggggaacgtgtttagttgttcagtgatgcatgaagccctgcacaatcattacactcagaagagcctgtccctgtctcccggcaaatgaggatcc

SEQ ID NO: 10 - protein, 261 aa, Homo sapiens
  misc_feature (30)..(261): Final protein product
EFATMAVMAPRTLVLLLSGALALTQTWAGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLICLVKGFYPSDIAVEWESNGQPENRYMTWPPVLDSDGSFFLYSKLTVDKSRWQQGNVFSCSVMHEALHNHYTQKSLSLSPGK

SEQ ID NO: 11 - DNA, 1446 bp, Homo sapiens
gaattcgccaccatggccgtgatggctccaagaaccctggtcctgctgctgagtggggcactggctctgacacagacatgggccggggaagtccagctggtcgaaagcggaggaggactggtgcagccaggagggtctctgcgactgagttgcgccgcttcaggcttcaacatcaaggacacctacattcactgggtgcgccaggctcctggaaaaggcctggagtgggtggcacgaatctatccaaccaatggatacacacggtatgccgacagcgtgaagggccggttcaccattagcgcagatacttccaaaaacaccgcctacctgcagatgaacagcctgcgagccgaagataccgctgtgtactattgcagtcggtggggaggcgacggcttctacgctatggattattgggggcagggaacactggtcactgtgagctccgcatctactaaggggcctagtgtgtttccactggccccctctagtaaatccacatctgggggaactgcagccctgggatgtctggtgaaggactatttcccagagcccgtcacagtgagttggaactcaggcgccctgacttccggggtccatacctttcctgctgtgctgcagtcaagcggcctgtactctctgtcctctgtggtcacagtgccaagttcaagcctggggacccagacatatatctgcaacgtgaatcacaagccaagcaatactaaagtcgacaagaaagtggaacccaagagctgtgataaaactcatacctgcccaccttgtcctgcaccagagctgctgggaggaccatccgtgttcctgtttccacccaagcctaaagacaccctgatgatttccaggaccccagaagtcacatgcgtggtcgtggacgtgtctcacgaggaccccgaagtcaagttcaactggtacgtggatggcgtcgaggtgcataatgccaagacaaaacccagggaggaacagtacaactcaacatatcgcgtcgtgagcgtcctgactgtgctgcaccaggactggctgaacggcaaggagtataagtgcaaagtgagcaataaggctctgcccgcacctatcgagaaaaccattagcaaggctaaagggcagcctagagaaccacaggtctacgtgctgcctccaagcagggacgagctgacaaagaaccaggtctccctgctgtgtctggtgaaagggttctatcccagtgatatcgcagtggagtgggaatcaaatggacagcctgaaaacaattacctgacctggccccctgtgctggacagcgatggcagcttcttcctgtattccaagctgacagtggataaatctcggtggcagcagggcaacgtctttagttgttcagtgatgcatgaggccctgcacaatcattacacccagaagagcctgtccctgtctcccggcaaatgaggatcc

SEQ ID NO: 12 - protein, 479 aa, Homo sapiens
  misc_feature (30)..(479): Final protein product
EFATMAVMAPRTLVLLLSGALALTQTWAGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 13 - DNA, 738 bp, Homo sapiens
gaattcgccactatggccgtgatggcacctagaaccctggtcctgctgctgtccggggcactggcactgactcagacttgggctggggatattcagatgacccagtcccctagctccctgtccgcttctgtgggcgacagggtcactatcacctgccgcgcatctcaggatgtgaacaccgcagtcgcctggtaccagcagaagcctgggaaagctccaaagctgctgatctacagtgcatcattcctgtattcaggagtgcccagccggtttagcggcagcagatctggcaccgacttcacactgactatctctagtctgcagcctgaggattttgccacatactattgccagcagcactataccacaccccctactttcggccaggggaccaaagtggagatcaagcgaactgtggccgctccaagtgtcttcatttttccacccagcgacgaacagctgaaatccggcacagcttctgtggtctgtctgctgaacaacttctaccccagagaggccaaagtgcagtggaaggtcgataacgctctgcagagtggcaacagccaggagagcgtgacagaacaggactccaaagattctacttatagtctgtcaagcaccctgacactgagcaaggcagactacgaaaagcataaagtgtatgcctgtgaggtgacccatcaggggctgtcttctcccgtgaccaagtctttcaaccgaggcgaatgttgaggatcc

SEQ ID NO: 14 - protein, 243 aa, Homo sapiens
  misc_feature (30)..(243): Final protein product
EFATMAVMAPRTLVLLLSGALALTQTWAGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - DNA, 792 bp, Homo sapiens
gaattcgccaccatggctgtgatggctccacgcaccctggtcctgctgctgtccggggcactggcactgactcagacttgggctggggaacctaaaagcagcgacaagacccacacatgccccccttgtccagctccagaactgctgggaggaccaagcgtgttcctgtttccacccaagcccaaagatacactgatgatcagccgaactcccgaggtcacctgcgtggtcgtggacgtgtcccacgaggaccccgaagtcaagttcaactggtacgtggacggcgtcgaagtgcataatgcaaagactaaaccacgggaggaacagtacaactctacatatagagtcgtgagtgtcctgactgtgctgcatcaggattggctgaacggcaaagagtataagtgcaaagtgtctaataaggccctgcctgctccaatcgagaaaactattagtaaggcaaaagggcagcccagggaacctcaggtctacgtgctgcctccaagtcgcgacgagctgaccaagaaccaggtctcactgctgtgtctggtgaaaggattctatccttccgatattgccgtggagtgggaatctaatggccagccagagaacaattacctgacctggccccctgtgctggacagcgatgggtccttctttctgtattcaaagctgacagtggacaaaagcagatggcagcagggaaacgtctttagctgttccgtgatgcacgaagccctgcacaatcattacacccagaagtctctgagtctgtcacctggcaaatgaggatcc

SEQ ID NO: 16 - protein, 261 aa, Homo sapiens
  misc_feature (30)..(261): Final protein product
EFATMAVMAPRTLVLLLSGALALTQTWAGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 17 - DNA, 1446 bp, Homo sapiens; identical to SEQ ID NO: 11

SEQ ID NO: 18 - protein, 479 aa, Homo sapiens; identical to SEQ ID NO: 12
  misc_feature (30)..(479): Final protein product

SEQ ID NO: 19 - DNA, 738 bp, Homo sapiens; identical to SEQ ID NO: 13

SEQ ID NO: 20 - protein, 243 aa, Homo sapiens; identical to SEQ ID NO: 14
  misc_feature (30)..(243): Final protein product

SEQ ID NO: 21 - DNA, 792 bp, Homo sapiens
gaattcgccaccatggccgtgatggcacctagaaccctggtcctgctgctgagcggggcactggcactgacacagacttgggctggggaacctaagagcagcgacaagactcacacctgcccaccttgtccagcaccagaactgctgggaggaccaagcgtgttcctgtttccacccaagcccaaagataccctgatgatcagccgaacacccgaagtgacttgcgtggtcgtggacgtgtcccacgaggaccccgaagtcaagttcaactggtacgtggacggcgtcgaagtgcataatgctaagacaaaaccacgggaggaacagtacaactctacttatagagtcgtgagtgtcctgaccgtgctgcatcaggattggctgaacggcaaagagtataagtgcaaagtgtctaataaggccctgcctgctccaatcgagaaaaccattagtaaggctaaagggcagcccagggaacctcaggtctacgtgtatcctccaagtcgcgacgagctgaccaagaaccaggtctcactgacatgtctggtgaaaggattttacccttccgatattgcagtggagtgggaatctaatggccagccagagaacaattataagaccacaccccctgtgctggacagcgatgggtccttcgcactggtctcaaagctgacagtggacaaaagcagatggcagcagggaaacgtctttagctgttccgtgatgcacgaagccctgcacaatcattacactcagaagtctctgagtctgtcacctggcaaatgaggatcc

SEQ ID NO: 22 - protein, 261 aa, Homo sapiens
  misc_feature (30)..(261): Final protein product
EFATMAVMAPRTLVLLLSGALALTQTWAGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 23 - DNA, 1446 bp, Homo sapiens
gaattcgccaccatggccgtgatggctcctagaaccctggtgctgctgctgtctggagctctggctctgactcagacctgggctggagaggtgcagctggtggaaagcggaggaggactggtgcagccaggaggatctctgcgactgagttgcgccgcttcaggattcaacatcaaggacacctacattcactgggtgcgacaggctccaggaaaaggactggagtgggtggctcgaatctatcccactaatggatacacccggtatgccgactccgtgaaggggaggtttactattagcgccgatacatccaaaaacactgcttacctgcagatgaacagcctgcgagccgaagataccgctgtgtactattgcagtcgatggggaggagacggattctacgctatggattattggggacaggggaccctggtgacagtgagctccgcctctaccaagggccccagtgtgtttcccctggctccttctagtaaatccacctctggagggacagccgctctgggatgtctggtgaaggactatttccccgagcctgtgaccgtgagttggaactcaggcgccctgacaagcggagtgcacacttttcctgctgtgctgcagtcaagcgggctgtactccctgtcctctgtggtgacagtgccaagttcaagcctgggcacacagacttatatctgcaacgtgaatcataagccctcaaatacaaaagtggacaagaaagtggagcccaagagctgtgataagacccacacctgccctccctgtccagctccagaactgctgggaggacctagcgtgttcctgtttccccctaagccaaaagacactctgatgatttccaggactcccgaggtgacctgcgtggtggtggacgtgtctcacgaggaccccgaagtgaagttcaactggtacgtggatggcgtggaagtgcataatgctaagacaaaaccaagagaggaacagtacaactccacttatcgcgtcgtgagcgtgctgaccgtgctgcaccaggactggctgaacgggaaggagtataagtgcaaagtcagtaataaggccctgcctgctccaatcgaaaaaaccatctctaaggccaaaggccagccaagggagccccaggtgtacacactgccacccagcagagacgaactgaccaagaaccaggtgtccctgacatgtctggtgaaaggcttctatcctagtgatattgctgtggagtgggaatcaaatggacagccagagaacaattacaagaccacacctccagtgctggacagcgatggcagcttcttcctgtattccaagctgacagtggataaatctcgatggcagcaggggaacgtgtttagttgttcagtgatgcatgaagccctgcacaatcattacactcagaagagcctgtccctgtctcccggcaaatgaggatcc

SEQ ID NO: 24 - protein, 479 aa, Homo sapiens
  misc_feature (30)..(479): Final protein product
EFATMAVMAPRTLVLLLSGALALTQTWAGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - DNA, 738 bp, Homo sapiens
gaattcgccactatggctgtgatggcccctaggaccctggtgctgctgctgtccggagctctggctctgactcagacctgggctggagacatccagatgacccagtctccatcctccctgtctgcatctgtaggagacagagtcaccatcacttgccgggcaagtcaggacgttaacaccgctgtagcttggtatcagcagaaaccagggaaagcccctaagctcctgatctattctgcatcctttttgtacagtggggtcccatcaaggttcagtggcagtcgatctgggacagatttcactctcaccatcagcagtctgcaacctgaagattttgcaacttactactgtcaacagcattacactaccccacccactttcggccaagggaccaaagtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaagagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgaggatcc

SEQ ID NO: 26 - protein, 243 aa, Homo sapiens; identical to SEQ ID NO: 14
  misc_feature (30)..(243): Final protein product

SEQ ID NO: 27 - DNA, 1446 bp, Homo sapiens
gaattcgccaccatggccgtgatggctcctagaaccctggtgctgctgctgtctggagctctggctctgactcagacctgggctggagaggtgcagctggtggaaagcggaggaggactggtgcagccaggaggatctctgcgactgagttgcgccgcttcaggattcaacatcaaggacacctacattcactgggtgcgacaggctccaggaaaaggactggagtgggtggctcgaatctatcccactaatggatacacccggtatgccgactccgtgaaggggaggtttactattagcgccgatacatccaaaaacactgcttacctgcagatgaacagcctgcgagccgaagataccgctgtgtactattgcagtcgatggggaggagacggattctacgctatggattattggggacaggggaccctggtgacagtgagctccgcctctaccaagggccccagtgtgtttcccctggctccttctagtaaatccacctctggagggacagccgctctgggatgtctggtgaaggactatttccccgagcctgtgaccgtgagttggaactcaggcgccctgacaagcggagtgcacacttttcctgctgtgctgcagtcaagcgggctgtactccctgtcctctgtggtgacagtgccaagttcaagcctgggcacacagacttatatctgcaacgtgaatcataagccctcaaatacaaaagtggacaagaaagtggagcccaagagctgtgataagacccacacctgccctccctgtccagctccagaactgctgggaggacctagcgtgttcctgtttccccctaagccaaaagacactctgatgatttccaggactcccgaggtgacctgcgtggtggtggacgtgtctcacgaggaccccgaagtgaagttcaactggtacgtggatggcgtggaagtgcataatgctaagacaaaaccaagagaggaacagtacaactccacttatcgcgtcgtgagcgtgctgaccgtgctgcaccaggactggctgaacgggaaggagtataagtgcaaagtcagtaataaggccctgcctgctccaatcgaaaaaaccatctctaaggccaaaggccagccaagggagccccaggtgtacgtgtacccacccagcagagacgaactgaccaagaaccaggtgtccctgacatgtctggtgaaaggcttctatcctagtgatattgctgtggagtgggaatcaaatggacagccagagaacaattacaagaccacacctccagtgctggacagcgatggcagcttcgccctggtgtccaagctgacagtggataaatctcgatggcagcaggggaacgtgtttagttgttcagtgatgcatgaagccctgcacaatcattacactcagaagagcctgtccctgtctcccggcaaatgaggatcc

SEQ ID NO: 28 - protein, 479 aa, Homo sapiens
  misc_feature (30)..(479): Final protein product
EFATMAVMAPRTLVLLLSGALALTQTWAGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 29 - DNA, 738 bp, Homo sapiens; identical to SEQ ID NO: 25

SEQ ID NO: 30 - protein, 243 aa, Homo sapiens; identical to SEQ ID NO: 14
  misc_feature (30)..(243): Final protein product

SEQ ID NO: 31 - DNA, 1446 bp, Homo sapiens
gaattcgccaccatggccgtgatggctcctagaaccctggtgctgctgctgtctggagctctggctctgactcagacctgggctggagaggtgcagctggtggaaagcggaggaggactggtgcagccaggaggatctctgcgactgagttgcgccgcttcaggattcaacatcaaggacacctacattcactgggtgcgacaggctccaggaaaaggactggagtgggtggctcgaatctatcccactaatggatacacccggtatgccgactccgtgaaggggaggtttactattagcgccgatacatccaaaaacactgcttacctgcagatgaacagcctgcgagccgaagataccgctgtgtactattgcagtcgatggggaggagacggattctacgctatggattattggggacaggggaccctggtgacagtgagctccgcctctaccaagggccccagtgtgtttcccctggctccttctagtaaatccacctctggagggacagccgctctgggatgtctggtgaaggactatttccccgagcctgtgaccgtgagttggaactcaggcgccctgacaagcggagtgcacacttttcctgctgtgctgcagtcaagcgggctgtactccctgtcctctgtggtgacagtgccaagttcaagcctgggcacacagacttatatctgcaacgtgaatcataagccctcaaatacaaaagtggacaagaaagtggagcccaagagctgtgataagacccacacctgccctccctgtccagctccagaactgctgggaggacctagcgtgttcctgtttccccctaagccaaaagacactctgatgatttccaggactcccgaggtgacctgcgtggtggtggacgtgtctcacgaggaccccgaagtgaagttcaactggtacgtggatggcgtggaagtgcataatgctaagacaaaaccaagagaggaacagtacaactccacttatcgcgtcgtgagcgtgctgaccgtgctgcaccaggactggctgaacgggaaggagtataagtgcaaagtcagtaataaggccctgcctgctccaatcgaaaaaaccatctctaaggccaaaggccagccaagggagccccaggtgtacgtgctgccacccagcagagacgaactgaccaagaaccaggtgtccctgctgtgtctggtgaaaggcttctatcctagtgatattgctgtggagtgggaatcaaatggacagccagagaacaattacctgacctggcctccagtgctggacagcgatggcagcttcttcctgtattccaagctgacagtggataaatctcgatggcagcaggggaacgtgtttagttgttcagtgatgcatgaagccctgcacaatcattacactcagaagagcctgtccctgtctcccggcaaatgaggatcc

SEQ ID NO: 32 - protein, 479 aa, Homo sapiens; identical to SEQ ID NO: 12
  misc_feature (30)..(479): Final protein product

SEQ ID NO: 33 - DNA, 1563 bp, Homo sapiens
gaattcgccaccatggctgtgatggctcctagaacactggtcctgctgctgtccggggcactggcactgactcagacttgggccgggcaggtccagctggtgcagagcggggcagaggtcaagaaacccggagaaagtctgaagatctcatgcaaagggagtggatactcattcaccagctattggattgcctgggtgaggcagatgcctggcaaggggctggaatacatgggcctgatctatccaggggacagcgatacaaaatactccccctctttccagggccaggtcacaatttccgtggacaagagtgtctcaactgcttatctgcagtggagctccctgaaacctagcgattccgcagtgtacttttgtgccaggcacgacgtcgggtattgcacagatcgcacttgtgcaaagtggccagagtggctgggagtgtggggacagggaaccctggtcacagtgtctagtggaggaggaggctcaagcggaggaggctctggaggaggagggtctcagagtgtgctgactcagccaccttcagtcagcgcagctcctggacagaaggtgaccatctcctgctctggcagctctagtaacattggcaacaattacgtgagctggtatcagcagctgcctggcaccgccccaaagctgctgatctacgaccacacaaatcggcccgctggggtgcctgatagattcagtgggtcaaaaagcggaacctccgcttctctggcaattagcggctttcgctccgaggacgaagctgattactattgtgcatcttgggactacacactgagtggctgggtgttcggaggcgggactaagctgaccgtgctgggggcagccgaaccaaagtcaagcgataaaactcatacctgcccaccatgtcctgcaccagagctgctgggaggaccttccgtgttcctgtttcctccaaagccaaaagacaccctgatgatcagccgaacaccagaagtgacttgcgtggtcgtggacgtctcccacgaggaccccgaagtgaagtttaactggtacgtggatggcgtcgaggtgcataatgccaagaccaaaccccgagaggaacagtacaactcaacttatcgggtcgtgagcgtcctgaccgtgctgcaccaggactggctgaacgggaaagagtataagtgcaaagtgtctaataaggccctgcccgctcctatcgagaaaacaattagcaaggccaaaggccagccaagagaaccccaggtgtacactctgcccccttctagggacgagctgaccaagaaccaggtgagcctgacatgtctggtcaaaggattctatcccagtgatattgctgtggagtgggaatccaatggccagcctgaaaacaattacaagaccacaccacccgtgctggactccgatggatctttctttctgtattccaagctgactgtggataaatctcggtggcagcagggcaacgtgtttagttgttcagtcatgcatgaggccctgcacaatcattacacacagaagagcctgtccctgtctcccggcaaatgaggatcc

SEQ ID NO: 34 - protein, 518 aa, Homo sapiens
  misc_feature (30)..(518): Final protein product
EFATMAVMAPRTLVLLLSGALALTQTWAGQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEWLGVWGQGTLVTVSSGGGGSSGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - DNA, 1563 bp, Homo sapiens
gaattcgccaccatggccgtgatggcacctagaacactggtcctgctgctgagcggagccctggcactgacacagacttgggccggacaggtccagctggtgcagagcggggcagaggtcaagaaacccggagaaagtctgaagatctcatgcaaagggagtggatactcattcaccagctattggattgcctgggtgaggcagatgcctggcaaggggctggaatacatgggcctgatctatccaggggacagcgatacaaaatactccccctctttccagggccaggtcacaatttccgtggacaagagtgtctcaactgcctatctgcagtggagctccctgaaacctagcgattccgcagtgtacttttgtgccaggcacgacgtcgggtattgcacagatcgcacttgtgctaagtggccagagtggctgggagtgtggggacagggaaccctggtcacagtgtctagtggaggaggaggctcaagcggaggaggctctggaggaggagggtctcagagtgtgctgactcagccaccttcagtcagcgcagctcctggacagaaggtgaccatctcctgctctggcagctctagtaacattggcaacaattacgtgagctggtatcagcagctgcctggcaccgccccaaagctgctgatctacgaccacacaaatcggcccgctggggtgcctgatagattcagtgggtcaaaaagcggaacctccgcttctctggcaattagcggctttcgctccgaggacgaagctgattactattgtgcatcttgggactacacactgagtggctgggtgttcggaggcgggactaagctgaccgtgctgggggcagccgaaccaaagtcaagcgataaaactcatacctgcccaccatgtcctgcaccagagctgctgggaggaccttccgtgttcctgtttcctccaaagccaaaagacaccctgatgatcagccgaacaccagaagtgacttgcgtggtcgtggacgtctcccacgaggaccccgaagtgaagtttaactggtacgtggatggcgtcgaggtgcataatgccaagaccaaaccccgagaggaacagtacaactcaacttatcgggtcgtgagcgtcctgaccgtgctgcaccaggactggctgaacgggaaagagtataagtgcaaagtgtctaataaggccctgcccgctcctatcgagaaaacaattagcaaggcaaaaggccagccaagagaaccccaggtgtacacttatcccccttctagggacgagctgaccaagaaccaggtgagcctgacatgtctggtcaaaggattttaccccagtgatattgctgtggagtgggaatccaatggccagcctgaaaacaattataagaccacaccacccgtgctggactccgatggatctttcgctctggtgtccaagctgactgtcgataaatctcggtggcagcagggcaacgtgtttagttgttcagtcatgcatgaggcactgcacaatcattacacacagaagagcctgtccctgtctcccggcaaatgaggatcc

SEQ ID NO: 36 - protein, 518 aa, Homo sapiens [sequence omitted]

SEQ ID NO: 37 - DNA, 792 bp, Homo sapiens
gaattcgctacaatggccgtgatggcaccccgaacactggtcctgctgctgagcggcgcactggcactgacacagacttgggctggagaacctaagagctccgacaaaacccacacatgccccccttgtccagctccagaactgctgggaggaccatccgtgttcctgtttccacccaagcccaaagatacactgatgatctctcgaactcccgaggtcacctgcgtggtcgtggacgtcagtcacgaggaccccgaagtcaagttcaactggtacgtggacggcgtcgaagtgcataatgcaaagactaaaccacgggaggaacagtacaactcaacatatagagtcgtgagcgtcctgactgtgctgcatcaggattggctgaacggcaaggagtataagtgcaaagtgagcaataaggccctgcctgctccaatcgagaaaaccattagcaaggcaaaagggcagcccagggaacctcaggtgtacaccctgcctccaagccgcgacgagctgacaaagaaccaggtctccctgctgtgtctggtgaaaggattctatcctagtgatattgccgtggagtgggaatcaaatggccagccagagaacaattacatgacttggccccctgtgctggactctgatgggagtttctttctgtattccaagctgaccgtggacaaatctagatggcagcagggaaacgtcttttcttgtagtgtgatgcacgaagccctgcacaatcattacacacagaagtcactgagcctgtcccctggcaaatgaggatcc

SEQ ID NO: 38 - protein, 261 aa, Homo sapiens
  misc_feature (30)..(261): Final protein product
EFATMAVMAPRTLVLLLSGALALTQTWAGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYMTWPPVLDSDGSFFLYSKLTVDKSRWQQGNVFSCSVMHEALHNHYTQKSLSLSPGK

SEQ ID NO: 39 - DNA, 1440 bp, Homo sapiens
gaattcgccacaatggctgtgatggctccaagaaccctggtcctgctgctgtccggggctctggctctgactcagacctgggccggggaagtgcagctggtcgaatctggaggaggactggtgcagccaggagggtccctgcgcctgtcttgcgccgctagtggcttcacttttaccgactacaccatggattgggtgcgacaggcacctggaaagggcctggagtgggtcgccgatgtgaacccaaatagcggaggctccatctacaaccagcggttcaagggccggttcaccctgtcagtggaccggagcaaaaacaccctgtatctgcagatgaatagcctgcgagccgaagatactgctgtgtactattgcgcccggaatctggggccctccttctactttgactattgggggcagggaactctggtcaccgtgagctccgcctccaccaagggaccttctgtgttcccactggctccctctagtaaatccacatctgggggaactgcagccctgggctgtctggtgaaggactacttcccagagcccgtcacagtgtcttggaacagtggcgctctgacttctggggtccacacctttcctgcagtgctgcagtcaagcgggctgtacagcctgtcctctgtggtcaccgtgccaagttcaagcctgggaacacagacttatatctgcaacgtgaatcacaagccatccaatacaaaagtcgacaagaaagtggaacccaagtcttgtgataaaacccatacatgccccccttgtcctgcaccagagctgctgggaggaccaagcgtgttcctgtttccacccaagcctaaagatacactgatgattagtaggaccccagaagtcacatgcgtggtcgtggacgtgagccacgaggaccccgaagtcaagtttaactggtacgtggacggcgtcgaggtgcataatgccaagactaaacccagggaggaacagtacaacagtacctatcgcgtcgtgtcagtcctgacagtgctgcatcaggattggctgaacgggaaagagtataagtgcaaagtgagcaataaggctctgcccgcacctatcgagaaaacaatttccaaggcaaaaggacagcctagagaaccacaggtgtacgtgtatcctccatcaagggatgagctgacaaagaaccaggtcagcctgacttgtctggtgaaaggattctatccctctgacattgctgtggagtgggaaagtaatggccagcctgagaacaattacaagaccacaccccctgtgctggactcagatggcagcttcgcgctggtgagcaagctgaccgtcgacaaatcccggtggcagcaggggaatgtgtttagttgttcagtcatgcacgaggcactgcacaaccattacacccagaagtcactgtcactgtcaccagggtgaggatcc

SEQ ID NO: 40 - protein, 477 aa, Homo sapiens
  misc_feature (30)..(477): Final protein product
EFATMAVMAPRTLVLLLSGALALTQTWAGEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 41 - DNA, 792 bp, Homo sapiens; identical to SEQ ID NO: 15

SEQ ID NO: 42 - protein, 261 aa, Homo sapiens; identical to SEQ ID NO: 16
  misc_feature (30)..(261): Final protein product

SEQ ID NO: 43 - DNA, 738 bp, Homo sapiens
gaattcgccacaatggctgtgatggcacctagaacactggtcctgctgctgagcggggcactggcactgacacagacttgggccggggatattcagatgacccagtccccaagctccctgagtgcctcagtgggcgaccgagtcaccatcacatgcaaggcttcccaggatgtgtctattggagtcgcatggtaccagcagaagccaggcaaagcacccaagctgctgatctatagcgcctcctaccggtataccggcgtgccctctagattctctggcagtgggtcaggaacagactttactctgaccatctctagtctgcagcctgaggatttcgctacctactattgccagcagtactatatctacccatatacctttggccaggggacaaaagtggagatcaagaggactgtggccgctccctccgtcttcatttttcccccttctgacgaacagctgaaaagtggcacagccagcgtggtctgtctgctgaacaatttctaccctcgcgaagccaaagtgcagtggaaggtcgataacgctctgcagagcggcaacagccaggagtctgtgactgaacaggacagtaaagattcaacctatagcctgtcaagcacactgactctgagcaaggcagactacgagaagcacaaagtgtatgcctgcgaagtcacacatcaggggctgtcctctcctgtgactaagagctttaacagaggagagtgttgaggatcc

SEQ ID NO: 44 - protein, 243 aa, Homo sapiens
  misc_feature (30)..(243): Final protein product
EFATMAVMAPRTLVLLLSGALALTQTWAGDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - DNA, 792 bp, Homo sapiens; identical to SEQ ID NO: 21

SEQ ID NO: 46 - protein, 261 aa, Homo sapiens; identical to SEQ ID NO: 22
  misc_feature (30)..(261): Final protein product

SEQ ID NO: 47 - DNA, 1440 bp, Homo sapiens
gaattcgccacaatggctgtgatggctccaagaaccctggtcctgctgctgtccggggctctggctctgactcagacctgggccggggaagtgcagctggtcgaatctggaggaggactggtgcagccaggagggtccctgcgcctgtcttgcgccgctagtggcttcacttttaccgactacaccatggattgggtgcgacaggcacctggaaagggcctggagtgggtcgccgatgtgaacccaaatagcggaggctccatctacaaccagcggttcaagggccggttcaccctgtcagtggaccggagcaaaaacaccctgtatctgcagatgaatagcctgcgagccgaagatactgctgtgtactattgcgcccggaatctggggccctccttctactttgactattgggggcagggaactctggtcaccgtgagctccgcctccaccaagggaccttctgtgttcccactggctccctctagtaaatccacatctgggggaactgcagccctgggctgtctggtgaaggactacttcccagagcccgtcacagtgtcttggaacagtggcgctctgacttctggggtccacacctttcctgcagtgctgcagtcaagcgggctgtacagcctgtcctctgtggtcaccgtgccaagttcaagcctgggaacacagacttatatctgcaacgtgaatcacaagccatccaatacaaaagtcgacaagaaagtggaacccaagtcttgtgataaaacccatacatgccccccttgtcctgcaccagagctgctgggaggaccaagcgtgttcctgtttccacccaagcctaaagatacactgatgattagtaggaccccagaagtcacatgcgtggtcgtggacgtgagccacgaggaccccgaagtcaagtttaactggtacgtggacggcgtcgaggtgcataatgccaagactaaacccagggaggaacagtacaacagtacctatcgcgtcgtgtcagtcctgacagtgctgcatcaggattggctgaacgggaaagagtataagtgcaaagtgagcaataaggctctgcccgcacctatcgagaaaacaatttccaaggcaaaaggacagcctagagaaccacaggtgtacgtgctgcctccatcaagggatgagctgacaaagaaccaggtcagcctgctgtgtctggtgaaaggattctatccctctgacattgctgtggagtgggaaagtaatggccagcctgagaacaattacctgacctggccccctgtgctggactcagatggcagcttctttctgtatagcaagctgaccgtcgacaaatcccggtggcagcaggggaatgtgtttagttgttcagtcatgcacgaggcactgcacaaccattacacccagaagtcactgtcactgtcaccagggtgaggatcc

SEQ ID NO: 48 - protein, 477 aa, Homo sapiens
  misc_feature (30)..(477): Final protein product
EFATMAVMAPRTLVLLLSGALALTQTWAGEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 49 - DNA, 738 bp, Homo sapiens; identical to SEQ ID NO: 43

SEQ ID NO: 50 - protein, 243 aa, Homo sapiens; identical to SEQ ID NO: 44
  misc_feature (30)..(243): Final protein product

SEQ ID NO: 51 - DNA, 1440 bp, Homo sapiens; identical to SEQ ID NO: 39

SEQ ID NO: 52 - protein, 477 aa, Homo sapiens; identical to SEQ ID NO: 40
  misc_feature (30)..(477): Final protein product

SEQ ID NO: 53 - DNA, 1440 bp, Homo sapiens; identical to SEQ ID NO: 47

SEQ ID NO: 54 - protein, 477 aa, Homo sapiens; identical to SEQ ID NO: 48
  misc_feature (30)..(477): Final protein product

SEQ ID NO: 55 - DNA, 738 bp, Homo sapiens; identical to SEQ ID NO: 43

SEQ ID NO: 56 - protein, 243 aa, Homo sapiens; identical to SEQ ID NO: 44
  misc_feature (30)..(243): Final protein product